(19) United States
(12) Patent Application Publication
(10) Pub. No.: US 2020/0061095 A1
(43) Pub. Date: Feb. 27, 2020

(54) Compounds for the Treatment of Systemic Insulin Resistance Disorders and the Use Thereof

(71) Applicant: Galectin Sciences, LLC, Norcross, GA (US)

(72) Inventors: Peter G. Traber, Alpharetta, GA (US); Eliezer Zomer, Newton, MA (US); Deirdre Slate, Berlin, MA (US); Joseph M. Johnson, Arlington, MA (US); Ryan George, Framingham, MA (US); Sharon Shechter, Andover, MA (US); Raphael Nir, Needham, MA (US)

(21) Appl. No.: 16/611,620

(22) PCT Filed: May 11, 2018
(86) PCT No.: PCT/US2018/032381

§ 371 Date: Nov. 7, 2019

Related U.S. Application Data
(60) Provisional application No. 62/505,544, filed on May 12, 2017.
(60) Provisional application No. 62/579,373, filed on Oct. 31, 2017.

(51) Int. Cl. A61K 31/7056; A61K 31/4439; A61P 3/10; A61K 45/06

(52) U.S. Cl. CPC A61K 31/7056; A61K 45/06; A61P 3/10; A61K 31/4439

(57) ABSTRACT

Aspects of the invention relate to novel synthetic compounds for treatment of metabolic diseases partially associated with systemic insulin resistance caused by Galectin proteins binding and inhibiting insulin and TGFb1 receptors causing physiological disturbances in the insulin pathways.

### RELATED APPLICATION(S)

This application claims the benefit of and priority to U.S. Provisional Application Ser. No. 62/579,343, filed Oct. 31, 2017, and to U.S. Provisional Application Ser. No. 62/505,544, filed May 12, 2017, the entire disclosures of each of which are incorporated herein by reference in their entireties.

### FIELD OF THE INVENTION

Aspects of the invention relate to compounds, pharmaceutical compositions, methods for the manufacturing of compounds and methods for treatment of metabolic disorders associated in-part with insulin resistance mediated at least in part by one or more galactose binding proteins also referred to as Galectins.

### BACKGROUND OF THE INVENTION

Galectins are a family of S-type lectins that bind beta-galactose glycan containing glycoproteins. To date, fifteen mammalian Galectins have been isolated. Galectins regulate different biological processes such as diabetes, inflammation, fibrogenesis, metabolic disorders, cancer progression, metastasis, apoptosis, and immune evasion.

### SUMMARY OF THE INVENTION

Aspects of the invention relate to compounds or compositions comprising a compound in an acceptable pharmaceutical carrier for parenteral or enteral administration, for use in therapeutic formulations. In some embodiments, the composition can be administered orally or topically or parenterally via an intravenous, or subcutaneous route.

Aspects of the invention relate to compounds, compositions and methods for treating metabolic disorders associated in-part with systemic insulin resistance. Aspects of the invention relate to compounds, compositions and methods for treating various disorders in which lectin proteins play a role in the pathogenesis, including but not limited to, treating of systemic insulin resistance by reversal of the Galectin-3 binding to the insulin receptor and enhancing sensitivity to insulin activity in various tissues.

Aspects of the invention relate to compounds, compositions and methods for the treatment of, but not limited to, systemic insulin resistance. In some embodiments, the systemic insulin resistance is associated with obesity where elevated galectin-3 interacts with insulin receptor. In some embodiments, treatment with compounds of this invention can restore sensitivity to insulin activity in various tissues.

Aspects of the invention relate to compounds, compositions and methods for the treatment of systemic insulin resistance associated with type 1 diabetes. Aspects of the invention relate to compounds, compositions and methods for the treatment of systemic insulin resistance associated with type 2 diabetes mellitus (T2DM). Aspects of the invention relate to compounds, compositions and methods for the treatment of systemic insulin resistance associated with obesity, gestational diabetes and prediabetes. In some embodiments, the compound restores sensitivity of cells to insulin activity. In some embodiments, the compound inhibits galectin-3 interaction with Insulin receptor, which interferes with insulin binding and cellular glucose uptake mechanism. Aspects of the invention relate to compounds, compositions and methods for the treatment of low-grade inflammation, due to elevated levels of free fatty acid and triglycerides that cause insulin resistance in skeletal muscle and liver which contributes to the development of atherosclerotic vascular diseases and NAFLD. Aspects of the invention relate to compounds, compositions and methods for the treatment of polycystic ovarian syndrome (PCOS) associated with obesity, insulin resistance, and the compensatory hyperinsulinemia. Aspects of the invention relate to compounds, compositions and methods for the treatment of diabetic nephropathy and glomerulosclerosis by attenuating integrin and TGFβ Receptor pathway in kidney chronic disease. In some embodiments, the compound can inhibit the overexpression of TGFβ receptor signaling system triggered by Insulin resistance in diabetic and cause decline in renal function, and can reverse the established lesions of diabetic glomerulopathy.

In some embodiments, the compound is administered with a pharmaceutically acceptable adjuvant, excipient, formulation carrier or combinations thereof. In some embodiments, the compound is administered with an active agent and a pharmaceutically acceptable adjuvant, excipient, formulation carrier or combinations thereof. In some embodiments, the compound is administered with one or more anti diabetic drug. In some embodiments, administration of the compound of the present invention and the active agent produces a synergistic effect.

Aspects of the invention relate to compounds, compositions and methods of treating systemic insulin resistance associated with obesity where elevated galectin-3 interacts with insulin receptor. In some embodiments, treatment with compounds of this invention can restore sensitivity to insulin activity in various tissues.

In some embodiments, the compounds or compositions of the invention that bind to insulin receptor (also identified as IR, INSR, CD220, HHF5).

Aspects of the invention relate to compounds or compositions or methods of treating diseases caused by disruption in the activity of TGFb1 (Transforming Growth Factor beta 1).

Aspects of the invention relate to compounds or compositions or methods of treating diseases associated with the Transforming Growth Factor Beta signaling pathway.

Aspects of the invention relate to compounds or compositions for the treatment of various disorders in which lectin proteins play a role in the pathogenesis, including but not limited to, chronic inflammatory diseases, fibrotic diseases, metabolic diseases and cancer. In some embodiments, the compound is capable of mimicking glycoprotein interactions with lectins or Galectin proteins which are known to modulate the pathophysiological pathways leading to inflammation, fibrogenesis, metabolic diseases, angiogenesis, and cancer progression, metastasis and immune evasion.

According to some aspects of the invention, the compounds comprise pyranosyl and/or furanosyl structures conjugated through an A-M spacer of at least 2 atoms comprising an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, Hydrazide —N(—H)—N(—H)— and/or an amino acid.

In some embodiments, the A-M spacer comprises an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, carbohydrazide —C(═O)—NH—NH—, sulfonohydrazide —S(═O)2-NH—NH—, or a phosphonic dihydrazide —P(═O)(—NH—NH2)(NH—NH—) spacer or any combination of the foregoing.

In some embodiments, the A-M spacer 2 or more atoms linked by single or double bond: C—C, C═C, C—P, C—N, C—O, N—C, N—N, N═N, N—S, N—P, S—N, P—O, O—P, S—C, S—N, S—S or combination thereof.

In some embodiments, the spacer is linked to the anomeric carbon of the pyranosyl and/or furanosyl structure.

In some embodiments, the compounds of this invention comprises mono, di or oligo pyranosyl and/or furanosyl structures conjugated through an amide or sulfonamide type linkage to organic substitutions with the general structures “R′-Gal-AM-R” where the “AM” presenting a linkage of at least two atoms such as, but not limited to, “amide”, “ester”, “methyl-sulfone” or “Sulfonamide” type linkage with R′ and R″ are organic substituents and are designated as “GalactoAmides” and/or “GalactoSulfonamides” and/or “GalactoCarbamide” with the general structures “R′-Gal-AM-R”.

In some embodiments, the compound comprises organic substituents. In some embodiments, specific aromatic substitutions can be linked to the galactose core or the “AM” linker of the anomeric carbon of the pyranosyl and/or furanosyl structures. Such aromatic substitutions can enhance the interaction of the compound with amino acid residues (e.g. Arginine, Tryptophan, Histidine, Glutamic acid etc. . . . ) composing the carbohydrate-recognition-domains (CRD) of the lectins or with amino acid residues in the CRD neighborhood and thus strengthen the association and binding specificity.

In some embodiments, the organic substituents comprise monosaccharides, disaccharides, oligosaccharides or a heteroglycoside such as iminosugar or thiosugar carbohydrates.

In some embodiments, the compound is a symmetric digalactoside, wherein the two galactosides are bound by an “AM” linker. Yet in other embodiments, the compound can be comprised of asymmetric carbohydrates. For example, each of the galactoside can have different aromatic or aliphatic substitutions or heteroatoms derivatives of galactose where the C5 oxygen is replaced with S (5-Thio-D-galactose) or N (5-imino-D-galactose).

Without being bound to the theory, it is believed that the compounds containing the “AM” spacer are metabolically stable while maintaining the chemical, physical and allosteric characteristics for specific interaction with lectins or Galectins known to recognize carbohydrates.

Aspects of the invention relate to a compound of formula 1 or a pharmaceutically acceptable salt or solvate thereof:

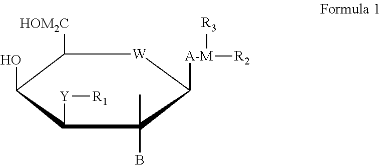

wherein A is selected from the group consisting of NRa, CRb, and PRc,

wherein M is selected from the group consisting of NRa, CRb, PRc, ORd, SRe amino acid, and hydrophobic hydrocarbons derivatives including heterocyclic substitutions of 3 or more atoms,

wherein Ra is selected from the group consisting of H, H2, CH3, COOH, NH2, COMe, halogen and combinations thereof,

wherein Rb is selected from the group consisting of H, H2, O, OH, CH3, COOH, NH2, COMe, halogen and combinations thereof,

wherein Rc is selected from the group consisting of O2, PO2, OH, halogen and combinations thereof,

wherein Rd is selected from the group consisting of H, CH3, and combination thereof,

wherein Re is selected from the group consisting of OH, O2, S, halogen and combinations thereof,

wherein B is OH, NH2, NHAc, or NH-alkyl wherein the alkyl comprises 1 to 18 Carbons,

wherein W is selected from the group consisting of O, S, CH2, NH, and Se,

wherein Y is selected from the group consisting of O, S, NH, CH2, Se, S, P, amino acid, and hydrophobic linear and cyclic hydrophobic hydrocarbons derivatives including heterocyclic substitutions of molecular weight of about 50-200 D and combinations thereof,

wherein R₁, R₂, and R₃ are independently selected from the group consisting of H, O2, CO, NH2, SO2, SO, PO2, PO, CH3, linear hydrocarbon, and cyclic hydrocarbon, and

wherein the hydrocarbon is one of a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, Deoxygalactose, substituted D-Galctose, C3-[1,2,3]-triaZol-1-yl-substituted D-galactose, hydrogen, an alkyl group, an alkenyl group, an aryl group, a heteroaryl group, and a heterocycle and derivatives, an amino group, a substituted amino group, an imino group, or a substituted imino group.

In some embodiments, A-M represents a spacer of at least 2 atoms comprising an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, Hydrazide —N(—H)—N(—H)— and amino acid, or combinations thereof,

In some embodiments, the A-M spacer comprises an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, carbohydrazide —C(═O)—NH—NH—, sulfonohydrazide —S(═O)2-NH—NH—, or a phosphonic dihydrazide —P(═O)(—NH—NH2)(NH—NH—) spacer or any combination of the foregoing.

In some embodiments, the A-M spacer comprises PO2 or PO2-PO2 bond linked to the anomeric carbon and to one or more atoms such as C or N or O or S. In some embodiments, C or N is linked to the anomeric carbon and PO2 or PO2-PO2 is linked to C or N.

In some embodiments, the A-M is methylamide linked R1, R2 is N′-methylamide-3,4-difluorobenzene and Y—R₁ is triazole-3-fluorobenzene

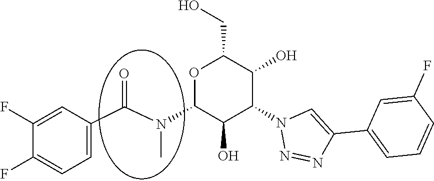

In some embodiments, the A-M spacer is linked to a galactose, a hydroxyl cyclohexane, an aromatic moiety, an alkyl group, an aryl group, an amine group, or amide group.

In some embodiments, the A-M spacer symmetrically links two galactosides or substituted derivatives thereof.

In some embodiments, the A-M spacer asymmetrically links two galactosides or substituted derivatives thereof.

In some embodiments, the anomeric carbon of the galactoside has a spacer of 2 or more atoms linked by single or double bond: C—C, C═C, C—P, C—N, C—O, N—C, N—N, N═N, N—S, N—P, S—N, P—O, O—P, or combination thereof.

Aspects of the invention relate to a compound or a pharmaceutically acceptable salt or solvate thereof having Formula 2:

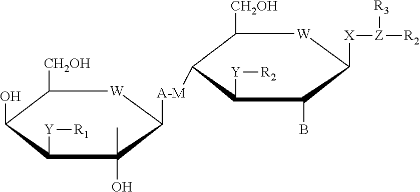

wherein A is selected from the group consisting of NRa, CRb, and PRc,

wherein M is selected from the group consisting of NRa, CRb, PRc, ORd, SRe amino acid, and hydrophobic hydrocarbons derivatives including heterocyclic substitutions of 3 or more atoms,

wherein Ra, Rb, Rc, Rd, Re are independently selected the group consisting of H, H2, O, O2, COOH, NH2, Halogen and combinations thereof,

wherein Ra is selected from the group consisting of H, H2, CH3, COOH, NH2, COMe, halogen and combinations thereof,

wherein Rb is selected from the group consisting of H, H2, O, OH, CH3, COOH, NH2, COMe, halogen and combinations thereof,

wherein Rc is selected from the group consisting of O2, PO2, OH, halogen and combinations thereof,

wherein Rd is selected from the group consisting of H, CH3, and combination thereof,

wherein Re is selected from the group consisting of OH, O2, S, halogen and combinations thereof,

wherein W is selected from the group consisting of O, S, CH2, NH, and Se,

wherein X is selected from the group consisting of O, N, S, CH2, NH, and PO2,

wherein Y and Z are independently selected from the group consisting of O, S, C, NH, CH2, Se, S, P, amino acid, and hydrophobic linear and cyclic hydrophobic hydrocarbons derivatives including heterocyclic substitutions of molecular weight of about 50-200 D and combinations thereof,

wherein R1, R2, R3, are independently selected from the group consisting of CO, O2, SO2, SO, PO2, PO, CH, Hydrogen, hydrophobic linear hydrocarbon, and hydrophobic cyclic hydrocarbon, wherein the hydrocarbon is one of:

a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted With an amino group, an alkyl group of at least 3 carbons substituted with both an amino and a carboxy group, an alkenyl group of at least 3 carbons substituted with both an amino and a carboxy group, and an alkyl group substituted with one or more halogens;
b) a phenyl group substituted with at least one car boxy group, a phenyl group substituted With at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group; and
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group;
e) saccharide; a substituted saccharide; D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives, an amino group, a substituted amino group, an imino group, or a substituted imino group.

In some embodiments, A-M represents a spacer of at least 2 atoms comprising an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, carbohydrazide —C(═O)—NH—NH—, sulfonohydrazide —S(═O)2-NH—NH—, and phosphonic dihydrazide —P(═O)(—NH—NH2)(NH—NH—) or combinations thereof.

In some embodiments, the A-M spacer of at least 2 atoms has a rotational freedom and length configured to allow an interaction of about 1 nM to about 50 μM to a galectin CRD epitope.

In some embodiments, the hydrophobic linear and cyclic hydrocarbons including heterocyclic substitutions have a molecule weight of about 50 to 200 D.

Aspects of the invention relate to a compound or a pharmaceutically acceptable salt or solvate thereof having formula of Table 1 and methods for using the same for the treatment of metabolic disorders associated in part with systemic insulin resistance.

Other aspects of the invention relate to methods for the treatment of metabolic disorders associated in part with systemic insulin resistance comprising administering to a subject in need thereof a therapeutically effective amount of a compound of Formula (3) or a pharmaceutically acceptable salt or solvate thereof

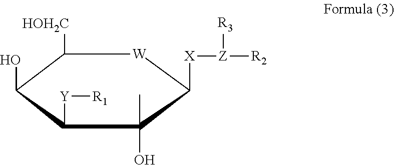

wherein X is S, O, or S(O2),
wherein W is selected from the group consisting of O, N, S, CH2, NH, and Se,
wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms, wherein R₁, R₂, and R₃ are independently selected from the group consisting of CO, O2, SO2, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide; a substituted saccharide, D-galactose, substituted D-galactose, C3-[1,2,3]-triazol-1-yl-substituted D-galactose, hydrogen, an alkyl group, an alkenyl group, an aryl group, a heteroaryl group, and a heterocycle and derivatives, an amino group, a substituted amino group, an imino group, and a substituted imino group.

Other aspects of the invention relate to methods for the treatment of metabolic disorders associated in part with systemic insulin resistance comprising administering to a subject in need thereof a therapeutically effective amount of a compound of Formula (4) or a pharmaceutically acceptable salt or solvate thereof

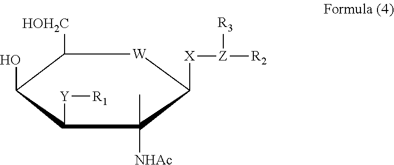

wherein X is S, O, or S(O2),

wherein W is selected from the group consisting of O, N, S, CH2, NH, and Se,

wherein Y is selected from the group consisting of O, S, NH, CH2, Se, S, S(O2), P(O2), amino acid, hydrophobic linear and cyclic hydrophobic hydrocarbon derivatives including heterocyclic substitutions of molecular weight of about 50-200 D and combinations thereof,

wherein Z is selected from the group consisting of O, S, NH, CH2, Se, P(O2), and hydrophobic hydrocarbons derivatives including heterocyclic substitutions of 3 or more atoms,

wherein R₁, R₂, and R₃ are independently selected from the group consisting of CO, O2, SO2, PO2, PO, CH, Hydrogen, and combination of these and, a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives; an amino group, a substituted amino group, an imino group, and a substituted imino group.

Other aspects of the invention relate to methods for the treatment of metabolic disorders associated in part with systemic insulin resistance comprising administering to a subject in need thereof a therapeutically effective amount of a compound of general Formula (5) or a pharmaceutically acceptable salt or solvate thereof

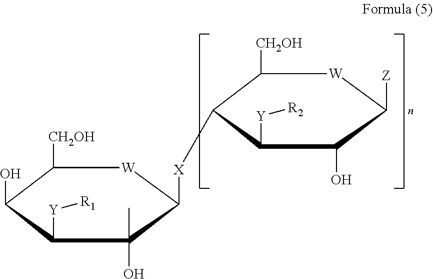

wherein X is S, O, or S(O2),
wherein W is selected from the group consisting of O, N, S, CH2, NH, and Se, wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms,
wherein n≤24,
wherein R₁ and R₂ are independently selected from the group consisting of CO, O2, SO2, SO, PO2, PO, CH, Hydrogen, combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one car boxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, or a substituted imino group.

Other aspects of the invention relate to methods for the treatment of metabolic disorders associated in part with systemic insulin resistance comprising administering to a subject in need thereof a therapeutically effective amount of a compound of general Formula (6) or a pharmaceutically acceptable salt or solvate thereof

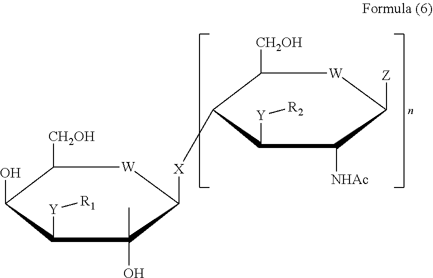

wherein X is S, O, or S(O2),
wherein W is selected from the group consisting of O, N, S, CH2, NH, and Se,
wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms,
wherein n≤24,
wherein R₁ and R₂ are independently selected from the group consisting of CO, O2, SO2, SO, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one car boxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, and a substituted imino group. In some embodiments, n=1. In other embodiments, n=2. Yet in other embodiments, n=3.

Other aspects of the invention relate to methods for the treatment of metabolic disorders associated in part with systemic insulin resistance comprising administering to a subject in need thereof a therapeutically effective amount of a compound of general Formula (7) or a pharmaceutically acceptable salt or solvate thereof

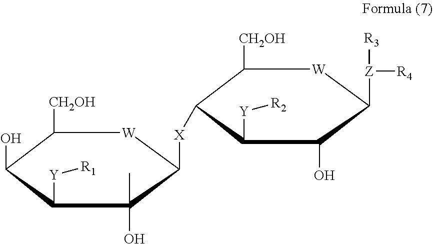

wherein X is S, O, S(O2), S—S, S—S(O2), S(O2)-S, O—S, S—O, O—S(O2), S(O2)-O, O—N(H), O—C(H2), O—C(O), O—C(H,OH), S—N(H), S—C(H2), S(O)—N(H), S(O2)-N(H), or O—P(O2),

wherein W is selected from the group consisting of O, N, S, CH2, NH, and Se,

wherein Y is selected from the group consisting of O, S, C, NH, CH2, Se, P, amino acid, hydrophobic linear and cyclic hydrophobic hydrocarbons derivatives including heterocyclic substitutions of molecular weight of about 50-200 D and combinations thereof,

wherein Z is selected from the group consisting of O, S, N, CH, Se, S, P, and hydrophobic hydrocarbons derivatives including heterocyclic substitutions of 3 or more atoms,

wherein R₁, R₂, R₃ and R₄ are independently selected from the group consisting of CO, O2, SO2, SO, PO2, PO, CH, Hydrogen, combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triazol-1-yl-substituted D-galactose, hydrogen, an alkyl group, an alkenyl group, an aryl group, a heteroaryl group, and a heterocycle and derivatives, an amino group, a substituted amino group, an imino group, and a substituted imino group.

Other aspects of the invention relate to methods for the treatment of metabolic disorders associated in part with systemic insulin resistance comprising administering to a subject in need thereof a therapeutically effective amount of a compound of general Formula (8) or a pharmaceutically acceptable salt or solvate thereof

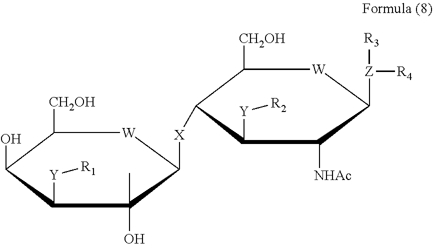

wherein X is S, O, S(O2), S—S, S—S(O2), S(O2)-S, O—S, S—O, O—S(O2), S(O2)-O, O—N(H), O—C(H2), O—C(O), O—C(H,OH), S—N(H), S—C(H2), S(O)—N(H), S(O2)-N(H), or O—P(O2),

wherein W is selected from the group consisting of O, N, S, CH2, NH, and Se,

wherein Y is selected from the group consisting of O, S, C, NH, CH2, Se, amino acid an combinations thereof,

wherein Z is selected from the group consisting of O, S, N, CH, Se, S, P, and hydrophobic hydrocarbons derivatives including heterocyclic substitutions of 3 or more atoms,

wherein R₁, R₂, R₃ and R₄ are independently selected from the group consisting of CO, O2, SO2, SO, PO2, PO, CH, Hydrogen, combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide; a substituted saccharide; D-galactose; substituted D-galactose, C3-[1,2,3]-triazol-1-yl-substituted D-galactose; hydrogen, an alkyl group, an alkenyl group, an aryl group, a heteroaryl group, and a heterocycle and derivatives; an amino group, a substituted amino group, an imino group, and a substituted imino group.

In some embodiments, the halogen is a fluoro, a chloro, a bromo or an iodo group.

In some embodiments, the compound is in a free form. In some embodiments, the free form is an anhydrate. In some embodiments, the free form is a solvate, such as a hydrate.

Some aspects of the invention relate to a compound of Formula (1), (2), (3), (4), (50, (6), (7), or (8) for use as a therapeutic agent in a mammal, such as a human.

Some aspects of the invention relate to a pharmaceutical composition comprising the compound of Formula (1), (2), (3), (4), (50, (6), (7), or (8) and optionally a pharmaceutically acceptable additive, such as carrier or excipient.

In some embodiments, the compounds of the present invention bind to one or more Galectins. In some embodiments, the compound binds to Galectin-3, and may bind to other Galectins e.g. Galectin-1, Galectin 8, and/or Galectin 9. In some embodiments, the compound binds to Galectin-3, Galectin-1, Galectin 8, and/or Galectin 9.

In some embodiments, the compounds of the present invention have high selectivity and affinity for Galectin-3. In some embodiments, the compounds of the present invention have an affinity of about 1 nM to about 50 μM for Galectin-3.

Aspects of the invention relate to compositions comprising the compound of the invention. In some embodiments, the composition comprises a therapeutically effective amount of the compound and a pharmaceutically acceptable adjuvant, excipient, formulation carrier or combinations thereof. In some embodiments, the composition comprises a therapeutically effective amount of the compound and of an anti-inflammatory drug, vitamin, pharmaceutical drug, nutraceutical drug, supplement, or combinations thereof.

Aspects of the invention relate to compositions or compounds that can be used in the treatment of diseases. Aspects of the invention relate to compositions or compounds that can be used in the treatment of metabolic diseases in which Galectins are at least in part involved in the pathogenesis. Other aspects of the invention relate to methods of treatment of a disease in a subject in need thereof.

In some embodiments, the composition or the compound can be used in the treatment of systemic insulin resistance due to obesity. In some embodiments, the composition or the compound can be used in the treatment of fatty liver with nonalcoholic steatohepatitis (NASH) with or without liver fibrosis, or cirrhosis.

In some embodiments, the invention relates to a method of treating systemic insulin resistance by reversal of the Galectin-3 binding to the insulin receptor and enhancing sensitivity to insulin activity in various tissues.

In some embodiments, the invention relates to a method of treating diseases due to disruption in the activity of TGFb1 (Transforming Growth Factor beta 1) by reversal of the Galectin-3 interaction with its receptor (TGFb1-Receptor) and recover normal regenerative activity in tissues.

In some embodiments, the invention relates to a method of treating diseases associated with the Transforming Growth Factor Beta-1 signaling pathway that involved many cellular and pathological processes in both the adult and embryo development including cell growth, cell differentiation, apoptosis, cellular homeostasis and other cellular functions.

In some embodiments, a therapeutically effective amount of the compound or of the composition can be compatible and effective in combination with a therapeutically effective amount of various anti-inflammatory drugs, vitamins, other pharmaceuticals and nutraceuticals drugs or supplement, or combinations thereof without limitation.

Some aspects of the present invention relate to a compound of Formula (1) or Formula (2) for use in a method for treating a disorder relating to the binding of a Galectin. Some aspects of the present invention relate to a compound of Formula (1) or Formula (2) for use in a method for treating a disorder relating to the binding of Galectin-3 to a ligand.

Some aspects of the present invention relate to a method for treatment of a disorder relating to the binding of a Galectin, such as Galectin-3 binding to an Insulin-Receptor or TGFb1-receptor in a human, wherein the method comprises administering a therapeutically effective amount of at least one compound of Formula (1), (2), (3), (4), (5), (6), (7), or (8) to a human in need thereof.

Some aspects of the invention relates to methods of treating insulin resistance, the method comprising administering to a subject in need thereof a composition comprising a therapeutically effective amount of the compound of Formula (1), (2), (3), (4), (5), (6), (7), or (8) or a pharmaceutically acceptable salt or solvate thereof.

In some embodiments, the compound can be used in conjunction with an active agent. In some embodiments, the active agent is an immunomodulatory, an anti-inflammatory drug, a vitamin, a nutraceutical drug, a supplement, or combinations thereof. In some embodiments, administration of the compound of the present invention and the active agent produces a synergistic effect.

Some aspects of the invention relate to a method of treating diseases due to disruption in the activity of TGFβ1 (Transforming Growth Factor beta 1) by reversal of the Galectin-3 interaction with its receptor (TGFβ1-Receptor) so as to recover normal regenerative activity in tissues.

Some aspects of the invention relate to a method of treating diseases associated with the Transforming Growth Factor Beta signaling pathway that involved many cellular and pathological processes in both the adult and embryo development including cell growth, cell differentiation, apoptosis, cellular homeostasis and other cellular functions.

Some aspects of the present invention relate to a method for treatment of a disorder relating to the binding of a Galectin, such as Galectin-3 binding to an Insulin-Receptor or TGFβ1-receptor in a human, wherein the method comprises administering a therapeutically effective amount of at least one compound of Formula (1) or Formula (2) to a subject in need thereof.

Some aspects of the present invention relate to a compound of Formula (1), (2), (3), (4), (5), (6), (7), or (8) or a pharmaceutically acceptable salt or solvate thereof for use in a method for treating a disorder relating to the binding of a galectin in a subject in need thereof. Some aspects of the present invention relate to a compound of Formula (1), (2), (3), (4), (5), (6), (7), or (8) or a pharmaceutically acceptable salt or solvate thereof for use in a method for treating a disorder relating to the binding of galectin-3 to a ligand in a subject in need thereof.

In some embodiments, the subject in need thereof is a mammal. In some embodiments, the subject in need thereof is a human.

Some aspects of the present invention relate to a method for treatment of a disorder relating to the binding of a galectin, such as galectin-3, to a ligand in a human, wherein the method comprises administering a therapeutically effective amount of at least one compound of Formula (1), (2), (3), (4), (5), (6), (7), or (8) or a pharmaceutically acceptable salt or solvate thereof to a human in need thereof. In some embodiments, the method of treatment is for systemic insulin resistance.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1A depicts a high-definition 3D structure of Galectin-3 Carbohydrate Recognition Domain (CRD) binding pocket with 3 potential sites of interaction.

FIG. 1B depicts the CRD pocket location in the Galectin-3 C-terminal with bound lactose unit.

FIG. 2 depicts a map of the Galectin-3 CRD site vicinity—potential cooperative amino-acids for enhanced binding.

FIG. 3A depicts in-silico 3D model predicted docking pose of a GalactoamideSuccinimide linked Compound according to some embodiments.

FIG. 3B depicts in-silico 3D model predicted docking pose of a GalactoAmide linked compound according to some embodiments.

FIGS. 4A-4W depicts the synthesis of GalactoAmide compounds according to some embodiments.

FIG. 5A depicts a Fluorescent Polarization Assay Format which detects compounds that bind specifically to the CRD according to some embodiments.

FIG. 5B depicts a Fluorescence Resonance Energy Transfer analytical assay (FRET Format) for screening compounds that inhibit Galectin-3 interaction with its Glycoprotein-ligand (for example TGFb1-Receptor FRET format) according to some embodiments.

FIG. 6A depicts the inhibition of Galectin binding moiety using a specific anti-Galectin-3 monoclonal antibodies binding assay (ELISA format) according to some embodiments.

FIG. 6B depicts a functional assay to screen compounds that inhibit the Galectin-3 interaction with its Glycoprotein-ligand (for example Insulin-Receptor ELISA format) according to some embodiments.

FIG. 7 provides examples of Compounds IC50 by Fluorescent Polarization—CRD specific assay of compounds according to some embodiments.

FIG. 8 provides examples of Compounds IC50 by the Insulin-Receptor-Galectin-3 ELISA format assays according to some embodiments.

FIG. 9 provides examples of Compounds IC50 by the TGFb1-Receptor-Galectin-3 ELISA format assay according to some embodiments.

FIG. 10 Illustrates inhibition of glucose uptake by Galectin-3 in 3T3L1 adipocytes model, and its reversal by a galactosulfonamides compound using the Promega Glucose Uptake-Glo™ Assay and protocol.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Citation of documents herein is not intended as an admission that any of the documents cited herein is pertinent prior art, or an admission that the cited documents are considered material to the patentability of the claims of the present application.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for additional factors not described, unless the context clearly dictates otherwise. In addition, throughout the specification, the meaning of “a,” “an,” and “the” include plural references.

Unless otherwise specified, all percentages expressed herein are weight/weight.

Aspects of the invention relate to compositions of mono, disaccharides and oligosaccharides of Galactose (or heteroglycoside) core bound to an A-M spacer of at least 2 atoms comprising an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, and/or an amino acid, on the anomeric carbon of the Galactose (or heteroglycoside).

In some embodiments, the A-M spacer comprises an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, carbohydrazide —C(═O)—NH—NH—, sulfonohydrazide —S(═O)2-NH—NH—, a phosphonic dihydrazide —P(═O)(—NH—NH2)(NH—NH—) spacer or any combination of the foregoing.

In some embodiments, the “AM” containing molecules render the compounds metabolically active while maintaining the chemical, physical and allosteric characteristics for specific interaction with lectins known to recognize carbohydrates. In some embodiments, the specific aromatic substitutions added to the galactose core further enhance the affinity of the “amide” bound pyranosyl and/or furanosyl structures by enhancing their interaction with amino acid residues (e.g. Arginine, Tryptophan, Histidine, Glutamic acid etc. . . . ) composing the carbohydrate-recognition-domains (CRD) of the lectins and thus strengthen the association and binding specificity.

### Galectins

Galectins (also known as galaptins or S-lectins) are a family of lectins which bind beta-galactoside. Galectin as a general name was proposed in 1994 for a family of animal lectins (Barondes, S. H., et al.: Galectins: a family of animal beta-galactoside-binding lectins. Cell 76, 597-598, 1994), The family is defined by having at least one characteristic carbohydrate recognition domain (CRD) with an affinity for beta-galactosides and sharing certain sequence elements. Further structural characterization segments the Galectins into three subgroups including: (1) Galectins having a single CRD, (2) Galectins having two CRDs joined by a linker peptide, and (3) a group with one member (Galectin-3) which has one CRD joined to a different type of N-terminal domain. The Galectin carbohydrate recognition domain is a beta-sandwich of about 135 amino acids. The two sheets are slightly bent with 6 strands forming the concave side, also called the S-face, and 5 strands forming the convex side, the F-face). The concave side forms a groove in which carbohydrate is bound (Leffler H, Carlsson S, Hedlund M, Qian Y, Poirier F (2004). “Introduction to Galectins”. Glycoconj. J. 19 (7-9): 433-40).

A wide variety of biological phenomena have been shown to be related to Galectins, including development, differentiation, morphogenesis, tumor metastasis, apoptosis, RNA splicing, and many others.

Generally, the carbohydrate domain binds to galactose contained glycan residues associated with glycoproteins. Galectins show an affinity for galactose residues attached to other organic compounds, such as in lactose [(P3-D-Galactosido)-D-glucose], N-acetyl-lactosamine, poly-N-acetyllactosamine, galactomannans, or fragments of pectins. However, it should be noted that galactose by itself does not bind to Galectins.

Plant polysaccharides like pectin and modified pectin have been shown to bind to Galectin proteins presumably on the basis of containing galactose residues that are presented in the context of a macromolecule, in this case a complex carbohydrate rather than a glycoprotein in the case of animal cells.

At least fifteen mammalian Galectin proteins have been identified which have one or two carbohydrate domains in tandem.

Galectin proteins are found in the intracellular space where they have been assigned a number of functions and they are also secreted into the extracellular space where they have different functions. In the extracellular space, Galectin proteins can have multiple functions that are mediated by their interaction with galactose containing glycoproteins including promoting interactions between glycoproteins that may modulate function or, in the case of integral membrane glycoprotein receptors, modification of cellular signaling (Sato et al “Galectins as danger signals in host-pathogen and host-tumor interactions: new members of the growing group of “Alarmins.” In “Galectins,” (Klyosov, et al eds.), John Wiley and Sons, 115-145, 2008, Liu et al “Galectins in acute and chronic inflammation,” Ann. N.Y. Acad. Sci. 1253: 80-91, 2012). Galectin proteins in the extracellular space can additionally promote cell-cell and cell matrix interactions (Wang et al., “Nuclear and cytoplasmic localization of Galectin-1 and Galectin-3 and their roles in pre-mRNA splicing.” In “Galectins” (Klyosov et al eds.), John Wiley and Sons, 87-95, 2008). In regards to intracellular space, Galectin functions appear to be more related to protein-protein interactions, although intracellular vesicle trafficking appears to be related to interaction with glycoproteins.

Galectins have been shown to have domains which promote homodimerization. Thus, Galectins are capable of acting as a “molecular glue” between glycoproteins. Galectins are found in multiple cellular compartments, including the nucleus and cytoplasm, and are secreted into the extracellular space where they interact with cell surface and extracellular matrix glycoproteins. The mechanism of molecular interactions can depend on the localization. While Galectins can interact with glycoproteins in the extracellular space, the interactions of Galectin with other proteins in the intracellular space generally occurs via protein domains. In the extracellular space the association of cell surface receptors may increase or decrease receptor signaling or the ability to interact with ligands.

Galectin proteins are markedly increased in a number of animal and human disease states, including but not limited to diseases associated with inflammation, fibrosis, autoimmunity, and neoplasia. Galectins have been directly implicated in the disease pathogenesis, as described below. For example, diseases states that may be dependent on Galectins include, but are not limited to, acute and chronic inflammation, metabolic disorders like systemic insulin resistance, allergic disorders, asthma, dermatitis, autoimmune disease, inflammatory and degenerative arthritis, immune-mediated neurological disease, fibrosis of multiple organs (including but not limited to liver, lung, kidney, pancreas, and heart), inflammatory bowel disease, atherosclerosis, heart failure, ocular inflammatory disease, a large variety of cancers.

In addition to disease states, Galectins are important regulatory molecules in modulating the response of immune cells to vaccination, exogenous pathogens and cancer cells.

One of skill in the art will appreciate that compounds that can bind to Galectins and/or alter Galectin's affinity for glycoproteins, reduce hetero- or homo-typic interactions between Galectins, or otherwise alter the function, synthesis, or metabolism of Galectin proteins may have important therapeutic effects in Galectin-dependent diseases.

Galectin proteins, such as Galectin-1 and Galectin-3 have been shown to be markedly increased in inflammation, fibrotic disorders, and neoplasia (Ito et al. “Galectin-1 as a potent target for cancer therapy: role in the tumor microenvironment”, Cancer Metastasis Rev. PMID: 22706847 (2012), Nangia-Makker et al. Galectin-3 binding and metastasis,” Methods Mol. Biol. 878: 251-266, 2012, Canesin et al. Galectin-3 expression is associated with bladder cancer progression and clinical outcome,” Tumor Biol. 31: 277-285, 2010, Wanninger et al. “Systemic and hepatic vein Galectin-3 are increased in patients with alcoholic liver cirrhosis and negatively correlate with liver function,” Cytokine. 55: 435-40, 2011). Moreover, experiments have shown that Galectins, particularly Galectin-1 (gal-1) and Galectin-3 (Galectin-3), are directly involved in the pathogenesis of these classes of disease (Toussaint et al., “Galectin-1, a gene preferentially expressed at the tumor margin, promotes glioblastoma cell invasion.”, Mol. Cancer. 11:32, 2012, Liu et al 2012, Newlaczyl et al., “Galectin-3—a jack-of-all-trades in cancer,” Cancer Lett. 313: 123-128, 2011, Banh et al., “Tumor Galectin-1 mediates tumor growth and metastasis through regulation of T-cell apoptosis,” Cancer Res. 71: 4423-31, 2011, Lefranc et al., “Galectin-1 mediated biochemical controls of melanoma and glioma aggressive behavior,” World J. Biol. Chem. 2: 193-201, 2011, Forsman et al., “Galectin 3 aggravates joint inflammation and destruction in antigen-induced arthritis,” Arthritis Reum. 63: 445-454, 2011, de Boer et al., “Galectin-3 in cardiac remodeling and heart failure,” Curr. Heart Fail. Rep. 7, 1-8, 2010, Ueland et al., “Galectin-3 in heart failure: high levels are associated with all-cause mortality,” Int. J. Cardiol. 150: 361-364, 2011, Ohshima et al., “Galectin 3 and its binding protein in rheumatoid arthritis,” Arthritis Rheum. 48: 2788-2795, 2003).

High levels of serum Galectin-3 have been shown to be associated with some human diseases, such as a more aggressive form of heart failure, which make identification of high-risk patients using Galectin-3 testing an important part of patient care. Galectin-3 testing may be useful in helping physicians determine which patients are at higher risk of hospitalization or death. For example, the BGM Galectin-3® Test is an in vitro diagnostic device that quantitatively measures Galectin-3 in serum or plasma and can be used in conjunction with clinical evaluation as an aid in assessing the prognosis of patients diagnosed with chronic heart failure. Measure of the concentration of endogenous protein Galectin-3 can be used to predict or monitor disease progression or therapeutic efficacy in patients treated with cardiac resynchronization therapy (see U.S. Pat. No. 8,672,857).

High levels of serum Galectin-3 have been shown to be associated with obesity and diabetes. Diabetes is an enduring disease which can be resolved but can be prevented by taking care. It is one of the commonly found metabolic syndromes in the world. Diabetes mellitus mainly associates with central nervous system and peripheral nervous system which are chronic complications. Diabetes mellitus is a commonly seen metabolic syndrome of diabetes where the body cannot use glucose and stores in blood which may damage kidneys, nerves, heart, eyes, and other complications.

### Insulin Resistance

Insulin resistance is a characteristic feature of patients with complications due to diabetes mellitus (T2DM) and is one of the defining clinical features in the Metabolic Syndrome (MetS). MetS is an array of biochemical and metabolic diseases that estimate to effect over 20% of adults (>20 years old) in the United States or approximately 50 million Americans. As the epidemic of obesity shows no signs of reversing, this number is likely to rise dramatically in the future.

Insulin resistance, the key feature of type 2 diabetes could develop in someone with type 1 diabetes designate clinically as Double diabetes. Someone with double diabetes will always have type 1 diabetes present but with complication of insulin resistance. The most common reason for developing insulin resistance is obesity and whilst type 1 diabetes is not itself brought on by obesity.

People with type 1 diabetes are able to become obese and suffer from insulin resistance as much as anyone else.

Insulin is a hormone which has diverse functions including stimulation of nutrient transport into cells, regulation of variety of enzymatic activity and regulation of energy homeostasis. These functions involve glucose metabolism through intracellular signaling pathways in the liver, adipose tissue and muscles. In the liver, insulin resistance leads to elevated hepatic glucose production. In adipose tissue insulin resistance affecting lipase activity leading to anti-lipolytic effecting free fatty acid efflux out of adipocytes and increasing circulating free fatty acids.

Recent studies indicate that Galectin-3 plasma levels are significantly elevated in human and animal obesity models.

In obesity, macrophages and other immune cells have been reported to recruited to insulin target tissues, and promoting a chronic inflammatory state and insulin resistance. Galectin-3 known to be mainly secreted by macrophages, may play a crucial role in this inflammation process thus it links inflammation to decrease in insulin sensitivity.

The insulin receptor is a transmembrane protein that is activated by bound insulin, IGF-I, IGF-II and belongs to the class of tyrosine kinase receptors. Insulin receptor plays a key role in the regulation of glucose homeostasis, that when dysfunction or metabolic impairment may result in a range of clinical manifestations including but not limited to diabetes. The insulin receptor is encoded by a single gene INSR, which during transcription may result in either IR-A or IR-B isoforms. Post-translational these isoform result in the formation of a proteolytically cleaved a and 3 subunits, which combine to form the final active ≈320 kDa transmembrane insulin receptor.

Insulin receptor and insulin interaction is checkpoint for a second pathway, the Ras-mitogen-activated protein kinase (MAPK) which mediates gene expression, and also affects the PI3K-AKT pathway that controls cell growth and differentiation. Insulin receptor substrate (IRS) is the common intermediate, which include four distinct family members, IRS1-4. Defects in insulin signaling typically involve insulin receptor substrate-1 (IRS1). Activation of the insulin receptor increase tyrosine phosphorylation of IRS1 which initiates signal transduction. However, when serine 307 is phosphorylated, signaling is diminished. Additional inflammation-related negative regulators of IR or IRS1 including the suppressor of cytokine signaling (Socs) may promote ubiquitylation, where ubiquitin, a small protein, is attached to another targeted protein changing their functionality and subsequent degradation, e.g. IRS inactivation.

Some aspects of the invention relate to compounds and use of compounds that inhibit Galectin-3 to treat insulin resistance.

### Galectin Inhibitors

Natural oligosaccharide ligands capable of binding to Galectin-1 and/or Galectin-3, for example, modified forms of pectins and galactomannan derived from Guar-gum have been described (see WO 2013040316, US 20110294755, WO 2015138438). Synthetic digalactosides like lactose, N-acetyllactosamine (LacNAc) and thiolactose effective against pulmonary fibrosis and other fibrotic disease (WO 2014067986 A1).

Advances in protein crystallography and availability of high definition 3D structure of the carbohydrate recognition domain (CRD) of many Galectins have generated many derivatives with enhanced affinity to the CRD having a greater affinity than galactose or lactose (WO 2014067986 A). These compounds were shown to be effective for treatment of an animal model of lung fibrosis which is thought to mimic human idiopathic pulmonary fibrosis (IPF). For example, a thio-digalactopyranosyl substituted with 3-fluorophenyl-2,3-triazol groups (TD-139) has been reported to bind to Galectin 3 and to be effective in in a mouse model of lung fibrosis. The compound required pulmonary administration using intra-tracheal instillation or nebulizers (see U.S. Pat. Nos. 8,703,720, 7,700,763, 7,638,623 U.S. Pat. No. 7,230,096).

Aspects of the invention relates to novel compounds that mimic the natural ligand of galectin proteins. In some embodiments, the compound mimics the natural ligand of galectin-3. In some embodiments, the compound mimics the natural ligand of galectin-1. In some embodiments, the compound mimics the natural ligand of galectin-8. In some embodiments, the compound mimics the natural ligand of galectin-9.

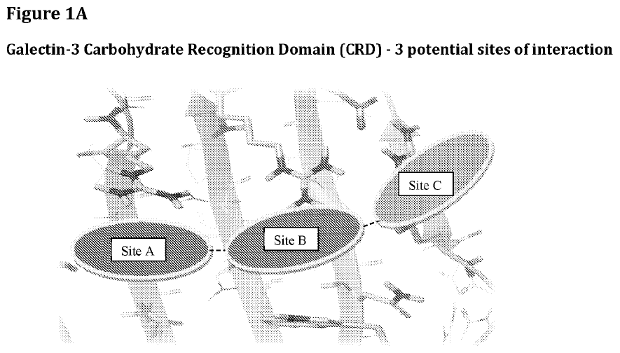

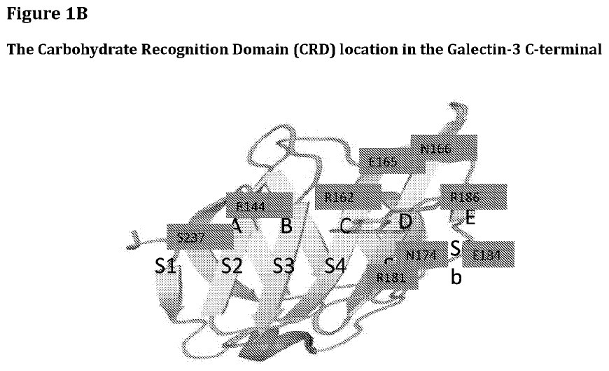

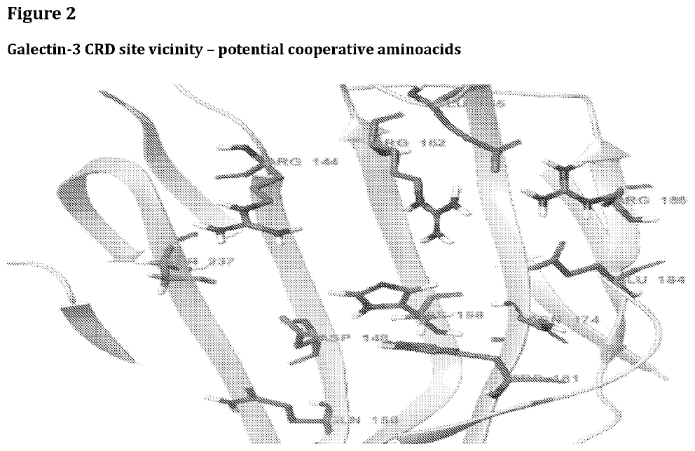

In some embodiments, the compound has a mono, di or oligomer structure composed of Galactose-AM core bound to the anomeric carbon on the galactose and which serves as a linker to the rest of the molecule. In some embodiments, the Galactose-AM core may be bound to other saccharide/amino acid/acids/group that bind Galectin CRD (as shown in FIG. 1A, 1B) in the high definition 3D structure of Galectin-3) and together can enhance the compound's affinity to the CRD. In some embodiments, the Galactose-AM core may be bound to other saccharide/amino acid/acid/group that bind in “site B” of the Galectin CRD (as shown in FIGS. 1A, 1B & FIG. 2 in the high definition 3D structure of Galectin-3) and together can enhance the compound's affinity to the CRD.

According to some aspects, the compounds can have substitutions that interact with site A and/or site C to further improve the association with the CRD and enhance their potential as a therapeutic targeted to Galectin-dependent pathology. In some embodiments, the substituents can be selected through in-silico analysis (computer assisted molecular modeling) as described herein. In some embodiments, the substituents can be further screened using binding assay with the Galectin protein of interest. For example, the compounds can be screened using a Galectin-3 binding assay and/or an in-vitro inflammatory and fibrotic model of activated cultured macrophages (see Macrophage polarization minireview, AbD Serotec).

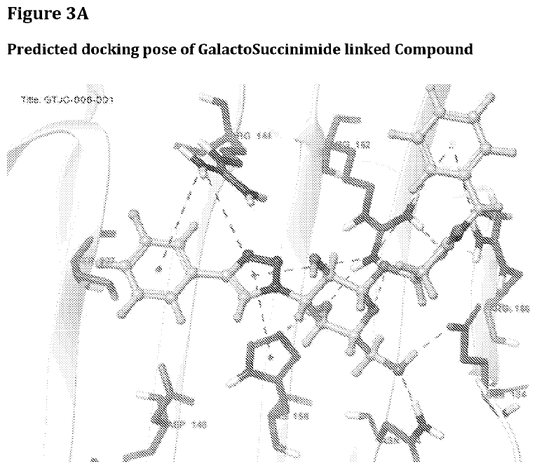

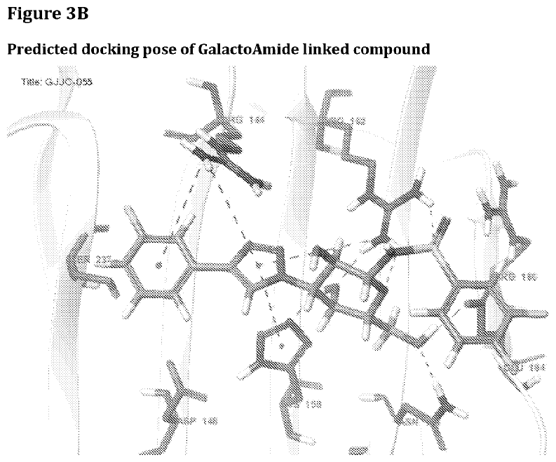

According to some aspects, the compounds comprise one or more specific substitutions of the core Galactose-AM. For example, the core Galactose-AM can be substituted with specific substituents that interact with residues located within the CRD. Such substituents can dramatically increase the association and potential potency of the compound as well as the ‘drugability’ characteristic (FIG. 3A, 3B)

### Galactoside-AM Compounds

Most “amide” and “sulfon” compounds, organic and inorganic, are readily absorbed from the diet and transported to the liver—the prime organ for metabolism. The general metabolism of “amide” compounds follows three major routes depending on the chemical properties, that is, redox-active “amide” compounds, precursors of methylamide and conjugation with amino acids.

### AM Spacer

Aspects of the invention relates to compounds comprising pyranosyl and/or furanosyl galactose structures bound to an “A-M” spacer on the anomeric carbon of the pyranosyl and/or furanosyl.

In some embodiments, A-M is representing a spacer of at least 2 atoms comprising an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, Hydrazide —N(—H)—N(—H)—, amino acid, or combinations thereof.

Without being bound to the theory, A-M is representing a spacer of at least 2 atoms that has more rotational freedom and length thus affording closer and tighter interaction to the galectin CRD epitope and surrounding aminoacids sites. Spacers like an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, carbohydrazide —C(═O)—NH—NH—, sulfonohydrazide —S(═O)2-NH—NH—, and phosphonic dihydrazide —P(═O)(—NH—NH2)(NH—NH—) augment the interaction with the galectin.

Aspects of the invention relates to compounds comprising pyranosyl and/or furanosyl galactose structures bound to an “amide” or “sulfon” type structures on the anomeric carbon of the pyranosyl and/or furanosyl. As used herein an amide bond refers to a C—N bond (R—C(O)—NH—R). In some embodiments, the amide bond can be a sulfonamide bond. In some embodiments, the sulfon bond can have the genera formula R—S(═O)₂—R′. As used herein an amide bond refers to a C—N bond (R—C(O)—NH—R). In some embodiments, the amide bond can be a N—SO2 (sulfonamide bond). or the genera R—N—S(═O)₂—R′. In some embodiments, the C—SO2 (sulfon bond) can have the genera formula R—C—S(═O)₂—R′.

In some embodiments, specific aromatic substitutions can be added to the galactose core or heteroglycoside core to further enhance the affinity of the “amide” bound pyranosyl and/or furanosyl structures. Such aromatic substitutions can enhance the interaction of the compound with amino acid residues (e.g. Arginine, Tryptophan, Histidine, Glutamic acid etc. . . . ) composing the carbohydrate-recognition-domains (CRD) of the lectins and thus strengthen the association and binding specificity.

In some embodiments, the compound comprises monosaccharides, disaccharides and oligosaccharides of galactose or a heteroglycoside core bound to an “amide” or “sulfon” atom on the anomeric carbon of the galactose or of the heteroglycoside.

In some embodiments, the compound is a symmetric digalactoside wherein the two galactosides are bound by one or more “amide” and/or “sulfon” bonds. In some embodiments, the compound is a symmetric digalactoside wherein the two galactosides are bound by one or more sulfonamide bonds. In some embodiments, the compound is a symmetric digalactoside wherein the two galactosides are bound by one or more “amide” bonds and wherein the “amide” is bound to the anomeric carbon of the galactose. In some embodiments, the compound is a symmetric digalactoside wherein the two galactosides are bound by one or more “amide” bonds and one or more sulfon bonds and wherein the “amide” is bound to the anomeric carbon of the galactose. Yet in other embodiments, the compound can be an asymmetric digalactoside. For example, the compound can have different aromatic or aliphatic substitutions on the galactose core.

In some embodiments, the compound is asymmetric galactoside wherein a single galactoside having one or more “amide” or “sulfon” on the anomeric carbon of the galactose. In some embodiments, the galactoside has one or more “amide” bound to the anomeric carbon of the galactose and one or more sulfur bound to the “amide”. In some embodiments, the compound can have different aromatic or aliphatic substitutions on the galactose core.

Without being bound to the theory, it is believed that the compounds containing the AM linkage render the compound metabolically stable while maintaining the chemical, physical and allosteric characteristics for specific interaction with lectins or galectins known to recognize carbohydrates. In some embodiments, the digalactoside or oligosaccharides of galactose of the invention are metabolically more stable than compounds having an O-glycosidic bond and resistant to most galactosidase digestion. In some embodiments, the digalactoside or oligosaccharides of galactose of the invention are metabolically more stable than compounds having an S-glycosidic bond.

Aspects of the invention relate to compounds having based on galactoside structure with “amide” type bridge [AM] to another galactose, hydroxyl cyclohexane, aromatic moiety, alkyl, aryl, amine, or amide group.

As used herein, the term “alkyl group” is meant to comprise from 1 to 12 carbon atoms, for example 1 to 7 or 1 to 4 carbon atoms. In some embodiments, the alkyl group may be a straight- or a branched-chain. In some embodiments, the alkyl group may also form a cycle comprising from 3 to 7 carbon atoms, preferably 3, 4, 5, 6, or 7 carbon atoms. Thus alkyl encompasses any of methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

As used herein, the term “alkenyl group” is meant to comprise from 2 to 12, for example 2 to 7 carbon atoms. The alkenyl group comprises at least one double bond. In some embodiments, the alkenyl group encompasses any any of vinyl, allyl, but-1-enyl, but-2-enyl, 2,2-direthylethenyl, 2,2-dimethylprop-1-enyl, pent-1-enyl, pent-2-enyl, 2,3-dimethylbut-1-enyl, hex-1-enyl, hex-2-enyl, hex-3-enyl, prop-1,2-dienyl, 4-methylhex-1-enyl, cycloprop-1-enyl group, and others.

As used herein, the term “alkoxy group” relates to an alkoxy group containing 1-12 carbon atoms, which may include one or more unsaturated carbon atoms. In some embodiments the alkoxy group contains 1 to 7 or 1 to 4 carbon atoms, which may include one or more unsaturated carbon atoms. Thus the term “alkoxy group” encompasses a methoxy group, an ethoxy group, a propoxy group, a isopropoxy group, a n-butoxy group, a sec-butoxy group, tert-butoxy group, pentoxy group, isopentoxy group, 3-methylbutoxy group, 2,2-dimethylpropoxy group, n-hexoxy group, 2-methylpentoxy group, 2,2-dimethylbutoxy group 2,3-dimethylbutoxy group, n-heptoxy group, 2-methylhexoxy group, 2,2-dimethylpentoxy group, 2,3-dimethylpentoxy group, cyclopropoxy group, cyclobutoxy group, cyclopentyloxy group, cyclohexyloxy group, cycloheptyloxy group, and 1-methylcyclopropyloxy group.

As used herein, the term “aryl group” is meant to comprise from 4 to 12 carbon atoms. Said aryl group may be a phenyl group or a naphthyl group. The above-mentioned groups may naturally be substituted with any other known substituents within the art of organic chemistry. The groups may also be substituted with two or more of the said substituents. Examples of substituents are halogen, alkyl, alkenyl, alkoxy, nitro, sulfo, amino, hydroxy, and carbonyl groups. Halogen substituents can be bromo, fluoro, iodo, and chloro. Alkyl groups are as defined above containing 1 to 7 carbon atoms. Alkenyl are as defined above containing 2 to 7 carbon atoms, preferably 2 to 4. Alkoxy is as defined below containing 1 to 7 carbon atoms, preferably 1 to 4 carbon atoms, which may contain an unsaturated carbon atom. Combinations of substituents can be present such as trifluoromethyl.

As used herein, the term “heteroaryl group” is meant to comprise any aryl group comprising from 4 to 18 carbon atoms, wherein at least one atom of the ring is a heteroatom, i.e. not a carbon. In some embodiments, the heteroaryl group may be a pyridine, or an indole group.

The above-mentioned groups may be substituted with any other known substituents within the art of organic chemistry. The groups may also be substituted with two or more of the substituents. Examples of substituents are halogen, alkoxy, nitro, sulfo, amino, hydroxy, and carbonyl groups. Halogen substituents can be bromo, fluoro, iodo, and chloro. Alkyl groups are as defined above containing 1 to 7 carbon atoms. Alkenyl are as defined above containing 2 to 7 carbon atoms, for example 2 to 4. Alkoxy is as defined below containing 1 to 7 carbon atoms, for example 1 to 4 carbon atoms, which may contain an unsaturated carbon atom.

Examples of substituents are

a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens. Halogens can be a fluoro, a chloro, a bromo or an iodo group.

b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,

c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group; and

d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group;

e) saccharide; a substituted saccharide, D-galactose, substituted D-galactose, C3-[1,2,3]-triazol-1-yl-substituted D-galactose, hydrogen, an alkyl group, an alkenyl group, an aryl group, a heteroaryl group, and a heterocycle and derivatives, an amino group, a substituted amino group, amino group, or a substituted imino group.

Wherein NRx is selected from the group consisting of hydrogen, an alkyl group, an alkenyl group, an aryl group, a heteroaryl group, and a heterocycle.

As used herein, the term “alkoxy group” relates to an alkoxy group containing 1-7 carbon atoms, which may include one or more unsaturated carbon atoms. In some embodiments the alkoxy group contains 1-4 carbon atoms, which may include one or more unsaturated carbon atoms. Thus the term “alkoxy group” encompasses a methoxy group, an ethoxy group, a propoxy group, a isopropoxy group, a n-butoxy group, a sec-butoxy group, tert-butoxy group, pentoxy group, isopentoxy group, 3-methylbutoxy group, 2,2-dimethylpropoxy group, n-hexoxy group, 2-methylpentoxy group, 2,2-dimethylbutoxy group 2,3-dimethylbutoxy group, n-heptoxy group, 2-methylhexoxy group, 2,2-dimethylpentoxy group, 2,3-dimethylpentoxy group, cyclopropoxy group, cyclobutoxy group, cyclopentyloxy group, cyclohexyloxy group, cycloheptyloxy group, and 1-methylcyclopropyloxy group.

### Monomeric Compound

In some embodiments, the compound or a pharmaceutically acceptable salt or solvate thereof has Formula 1

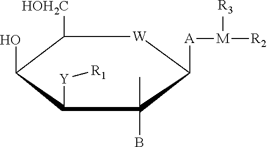

wherein A-M is representing a spacer of at least 2 atoms comprising an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, amino acid, Hydrazide —N(—H)—N(—H)—, or combinations thereof,
wherein A is selected from the group consisting of NRa, CRb, and PRc,
wherein M is selected from the group consisting of NRa, CRb, PRc, ORd, SRe amino acid, and hydrophobic hydrocarbons derivatives including heterocyclic substitutions of 3 or more atoms,
wherein Ra is selected from the group consisting of H, H2, CH3, COOH, NH2, COMe, halogen and combinations thereof,
wherein Rb is selected from the group consisting of H, H2, O, OH, CH3, COOH, NH2, COMe, halogen and combinations thereof,
wherein Rc is selected from the group consisting of O2, PO2, OH, halogen and combinations thereof,
wherein Rd is selected from the group consisting of H, CH3, and combination thereof,
wherein Re is selected from the group consisting of OH, O2, S, halogen and combinations thereof,
wherein B is OH, NH2, NHAc, or NH-alkyl, wherein the alkyl group comprises 1 to 18 Carbons,
wherein W is selected from the group consisting of O, S, CH2, NH, and Se,
wherein Y is selected from the group consisting of O, S, NH, CH2, Se, S, P, amino acid, and hydrophobic linear and cyclic hydrophobic hydrocarbons derivatives including heterocyclic substitutions of molecular weight of about 50-200 D and combinations thereof,
wherein R₁, R₂, and R₃ are independently selected from the group consisting of H, O2, CO, NH2, SO2, SO, PO2, PO, CH3, linear hydrocarbon, and cyclic hydrocarbon, and
wherein the hydrocarbon is one of a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, Deoxygalactose, substituted D-Galctose, C3-[1,2,3]-triaZol-1-yl-substituted D-galactose, hydrogen, an alkyl group, an alkenyl group, an aryl group, a heteroaryl group, and a heterocycle and derivatives, an amino group, a substituted amino group, an imino group, or a substituted imino group.

In some embodiments, the compound has the general formula (1), wherein AM-R1 is for example N′-methylamide-3,4-difluorobenzene, wherein Y—R1 is triazole-3-fluorobenzene

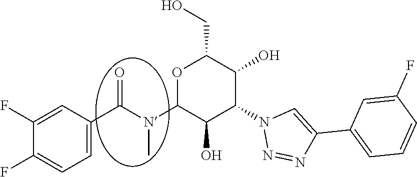

### Dimeric Compounds

In some embodiments, the compound is a dimeric-polyhydroxylated-cycloalkanes compound.

In some embodiments, the compound or a pharmaceutically acceptable salt or solvate thereof has Formula (2):

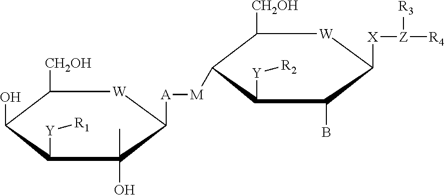

wherein A-M is representing a spacer of at least 2 atoms comprising an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, Hydrazide —N(—H)—N(—H)— or combinations thereof, wherein A is selected from the group consisting of NRa, CRb, and PRc,
wherein M is selected from the group consisting of NRa, CRb, PRc, ORd, SRe, amino acid, and hydrophobic hydrocarbons derivatives including heterocyclic substitutions of 3 or more atoms.
wherein Ra is selected from the group consisting of H, H2, CH3, COOH, NH2, COMe, halogen and combinations thereof,
wherein Rb is selected from the group consisting of H, H2, O, OH, CH3, COOH, NH2, COMe, halogen and combinations thereof,
wherein Rc is selected from the group consisting of O2, PO2, OH, halogen and combinations thereof,
wherein Rd is selected from the group consisting of H, CH3, and combination thereof,
wherein Re is selected from the group consisting of OH, O2, S, halogen and combinations thereof,
wherein B is OH, NH2, NHAc, or NH-alkyl of 1 to 18 Carbons,
wherein W is selected from the group consisting of O, S, CH2, NH, or Se
Wherein X is selected from the group consisting of O, N, S, CH2, NH, and PO2
wherein Y and Z are independently selected from the group consisting of O, S, C, NH, CH2, Se, S, P, amino acid, hydrophobic linear and cyclic hydrophobic hydrocarbons derivatives including heterocyclic substitutions of molecular weight of about 50-200 D and combinations thereof,
wherein R1, R2, R3, are independently selected from the group consisting of CO, O2, SO2, SO, PO2, PO, CH, Hydrogen, hydrophobic linear hydrocarbon, and hydrophobic cyclic hydrocarbon, wherein the hydrocarbon is one of:
a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted With an amino group, an alkyl group of at least 3 carbons substituted with both an amino and a carboxy group, an alkenyl group of at least 3 carbons substituted with both an amino and a carboxy group, and an alkyl group substituted with one or more halogens;
b) a phenyl group substituted with at least one car boxy group, a phenyl group substituted With at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group; and
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group,
e) saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, or a substituted imino group.

In some embodiments, the compound has the general Formula below

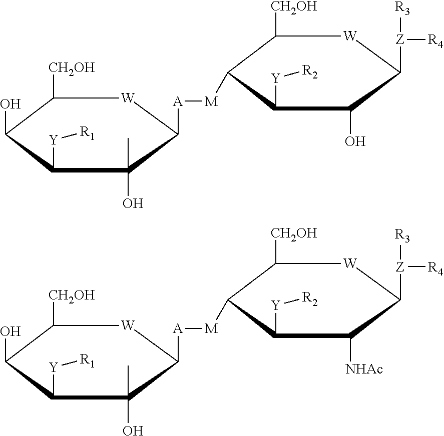

Wherein A-M is an amide, Sulfate, sulfonamide, a carbon ester and/or includes an aryl derivative like AM-Benzene-AM structure (Example 14, Scheme 6),
Wherein W is selected from the group consisting of O, N, S, CH2, NH, and Se,
Wherein Y and Z are independently selected from the group consisting of O, S, C, NH, CH2, NR, Se, or Amino acid,
Wherein R1, R2, R3, and R4 (Rx) are independently selected from the group consisting of CO, SO2, SO, PO2, PO, CH, Hydrogen, Hydrophobic linear and cyclic including Heterocyclic substitutions of molecular weight of 50-200 D including, but not limited to:
a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens;
b) a phenyl group substituted with at least one car boxy group, a phenyl group substituted With at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group; and
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group;
e) saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, amino group, or a substituted imino group.
Rx is selected from the group consisting of hydrogen, an alkyl group, an alkenyl group, an aryl group, a heteroaryl group, and a heterocycle.

As used herein, the term “alkyl group” relates to an alkyl group containing 1-7 carbon atoms, which may include one or more unsaturated carbon atoms. In some embodiments the alkyl group contains 1-4 carbon atoms, which may include one or more unsaturated carbon atoms. The carbon atoms in the alkyl group may form a straight or branched chain. The carbon atoms in said alkyl group may also form a cycle containing 3, 4, 5, 6, or 7 carbon atoms. Thus, the term “alkyl group” used herein encompasses methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

Without being bound to the theory, the galactose-Amide or sulfon based linker compounds described herein have an enhanced stability as its structure is less prone to hydrolysis (metabolism) and oxidation e.g. aromatic ring without substitutions, Carbon-Oxygen systems, Carbone-Nitrogen system etc.

In some embodiments, the compounds comprising pyranosyl and/or furanosyl galactose structures bound to an “amide” or “sulfon” type structures on the anomeric carbon of the pyranosyl and/or furanosyl. As used herein an amide bond refers to a C—N bond (R—C(O)—NH—R). In some embodiments, the amide bond can be a N—SO2 (sulfonamide bond) can have the genera formula R—N—S(═O)₂—R′. In some embodiments, the C—S(O2)-C (sulfon bond) can have the genera formula R—C—S(═O)₂—R′.

In some embodiments, specific aromatic substitutions can be added to the galactose core or heteroglycoside core to further enhance the affinity of the “amide” bound pyranosyl and/or furanosyl structures. Such aromatic substitutions can enhance the interaction of the compound with amino acid residues (e.g. Arginine, Tryptophan, Histidine, Glutamic acid etc. . . . ) composing the carbohydrate-recognition-domains (CRD) of the lectins and thus strengthen the association and binding specificity.

In some embodiments, the compound comprises monosaccharides, disaccharides and oligosaccharides of galactose or a heteroglycoside core bound to an “amide” or “sulfon” atom on the anomeric carbon of the galactose or of the heteroglycoside.

In some embodiments, the compound is a symmetric digalactoside wherein the two galactosides or galactose and a carbohydrate (like glucose) are bound by one or more “amide” and/or “sulfon” bonds. In some embodiments, the compound is a symmetric digalactoside wherein the two galactosides are bound by one or more “amide” or “sulfon” bonds. In some embodiments, the compound is a symmetric digalactoside wherein the two galactosides are bound by one or more “amide” bonds and wherein the “amide” is bound to the anomeric carbon of the galactose. In some embodiments, the compound is a symmetric digalactoside wherein the two galactosides are bound by one or more “amide” bonds and one or more sulfon bonds and wherein the “amide” is bound to the anomeric carbon of the galactose. Yet in other embodiments, the compound can be an asymmetric digalactoside. For example, the compound can have different aromatic or aliphatic substitutions on the galactose core.

In some embodiments, the compound is asymmetric galactoside wherein a single galactoside having one or more “amide” or “sulfon” on the anomeric carbon of the galactose. In some embodiments, the galactoside has one or more “amide” bound to the anomeric carbon of the galactose and one or more sulfur bound to the “amide”. In some embodiments, the compound can have different aromatic or aliphatic substitutions on the galactose core.

Without being bound to the theory, it is believed that the compounds containing the AM linkage render the compound metabolically stable while maintaining the chemical, physical and allosteric characteristics for specific interaction with lectins or Galectins known to recognize carbohydrates. In some embodiments, the digalactoside or oligosaccharides of galactose of the present invention are metabolically more stable than compounds having an O-glycosidic bond and resistant to most galactosidase digestion. In some embodiments, the digalactoside or oligosaccharides of galactose of the present invention are metabolically more stable than compounds having a single O or S-glycosidic bond.

Aspects of the invention relate to compounds having based on galactoside structure with “amide” bridge [AM] to another galactose, hydroxyl cyclohexane, aromatic moiety, alkyl, aryl, amine, or amide group.

As used herein, the term “alkyl group” is meant to comprise from 1 to 12 carbon atoms, for example 1 to 7 or 1 to 4 carbon atoms. In some embodiments, the alkyl group may be a straight- or a branched-chain. In some embodiments, the alkyl group may also form a cycle comprising from 3 to 7 carbon atoms, preferably 3, 4, 5, 6, or 7 carbon atoms. Thus, alkyl encompasses any of methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

As used herein, the term “alkenyl group” is meant to comprise from 2 to 12, for example 2 to 7 carbon atoms. The alkenyl group comprises at least one double bond. In some embodiments, the alkenyl group encompasses any any of vinyl, allyl, but-1-enyl, but-2-enyl, 2,2-dimethylethenyl, 2,2-dimethylprop-1-enyl, pent-1-enyl, pent-2-enyl, 2,3-dimethylbut-1-enyl, hex-1-enyl, hex-2-enyl, hex-3-enyl, prop-1,2-dienyl, 4-methylhex-1-enyl, cycloprop-1-enyl group, and others.

As used herein, the term “alkoxy group” relates to an alkoxy group containing 1-12 carbon atoms, which may include one or more unsaturated carbon atoms. In some embodiments the alkoxy group contains 1 to 7 or 1 to 4 carbon atoms, which may include one or more unsaturated carbon atoms. Thus the term “alkoxy group” encompasses a methoxy group, an ethoxy group, a propoxy group, a isopropoxy group, a n-butoxy group, a sec-butoxy group, tert-butoxy group, pentoxy group, isopentoxy group, 3-methylbutoxy group, 2,2-dimethylpropoxy group, n-hexoxy group, 2-methylpentoxy group, 2,2-dimethylbutoxy group 2,3-dimethylbutoxy group, n-heptoxy group, 2-methylhexoxy group, 2,2-dimethylpentoxy group, 2,3-dimethylpentoxy group, cyclopropoxy group, cyclobutoxy group, cyclopentyloxy group, cyclohexyloxy group, cycloheptyloxy group, and 1-methylcyclopropyloxy group.

As used herein, the term “aryl group” is meant to comprise from 4 to 12 carbon atoms. Said aryl group may be a phenyl group or a naphthyl group. The above-mentioned groups may naturally be substituted with any other known substituents within the art of organic chemistry. The groups may also be substituted with two or more of the said substituents. Examples of substituents are halogen, alkyl, alkenyl, alkoxy, nitro, sulfo, amino, hydroxy, and carbonyl groups. Halogen substituents can be bromo, fluoro, iodo, and chloro. Alkyl groups are as defined above containing 1 to 7 carbon atoms. Alkenyl are as defined above containing 2 to 7 carbon atoms, preferably 2 to 4. Alkoxy is as defined below containing 1 to 7 carbon atoms, preferably 1 to 4 carbon atoms, which may contain an unsaturated carbon atom. Combinations of substituents can be present such as trifluoromethyl.

As used herein, the term “heteroaryl group” is meant to comprise any aryl group comprising from 4 to 18 carbon atoms, wherein at least one atom of the ring is a heteroatom, i.e. not a carbon. In some embodiments, the heteroaryl group may be a pyridine, or an indole group.

The above-mentioned groups may be substituted with any other known substituents within the art of organic chemistry. The groups may also be substituted with two or more of the substituents. Examples of substituents are halogen, alkoxy, nitro, sulfo, amino, hydroxy, and carbonyl groups. Halogen substituents can be bromo, fluoro, iodo, and chloro. Alkyl groups are as defined above containing 1 to 7 carbon atoms. Alkenyl are as defined above containing 2 to 7 carbon atoms, for example 2 to 4. Alkoxy is as defined below containing 1 to 7 carbon atoms, for example 1 to 4 carbon atoms, which may contain an unsaturated carbon atom.

a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens. Halogens can be a fluoro, a chloro, a bromo or an iodo group.

b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,

c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group; and

d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group;

e) saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives; an amino group, a substituted amino group, amino group, or a substituted imino group.

Wherein NR5 is selected from the group consisting of hydrogen, an alkyl group, an alkenyl group, an aryl group, a heteroaryl group, and a heterocycle.

As used herein, the term “alkoxy group” relates to an alkoxy group containing 1-7 carbon atoms, which may include one or more unsaturated carbon atoms. In some embodiments the alkoxy group contains 1-4 carbon atoms, which may include one or more unsaturated carbon atoms. Thus the term “alkoxy group” encompasses a methoxy group, an ethoxy group, a propoxy group, a isopropoxy group, a n-butoxy group, a sec-butoxy group, tert-butoxy group, pentoxy group, isopentoxy group, 3-methylbutoxy group, 2,2-dimethylpropoxy group, n-hexoxy group, 2-methylpentoxy group, 2,2-dimethylbutoxy group 2,3-dimethylbutoxy group, n-heptoxy group, 2-methylhexoxy group, 2,2-dimethylpentoxy group, 2,3-dimethylpentoxy group, cyclopropoxy group, cyclobutoxy group, cyclopentyloxy group, cyclohexyloxy group, cycloheptyloxy group, and 1-methylcyclopropyloxy group.

In some embodiments, the compound is a dimeric or trimeric polyhydroxylated-cycloalkanes compound.

Aspect the present invention relates to a compound of Formula (1) or a pharmaceutically acceptable salt or solvate thereof:

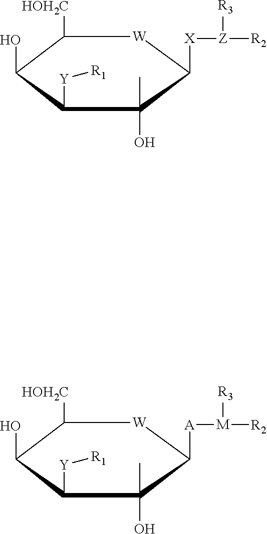

Wherein X is the first atom in the linkage selected from NH, NCH3, SO2, CH2, CHOH, or CHCH3,
Wherein Z is independently selected from O, S, NH, NCH3, SO2, CH2, CHOH, or CHCH3,
Wherein “A-M” represents an amide type linkage, e.g. amide, N′-methylamide, Sulfonamide, carbosulfon, or acetohydrazide linkage to the substitutions R₂ and R₃,
Wherein W is selected from the group consisting of O, N, S, CH2, NH, and Se,
Wherein Y is selected from the group consisting of O, S, C, NH, CH2, Se, and amino acid,
Wherein R₁, R₂, R₃, and R₄ are independently selected from the group consisting of H, CO, SO2, SO, PO2, PO, CH, galactose, carbohydrate, and/or hydrophobic linear and cyclic hydrophobic hydrocarbons derivative including heterocyclic substitutions of molecular weight of about 50-200 D.

Aspect the present invention relates to a compound of Formula (2) or a pharmaceutically acceptable salt or solvate thereof:

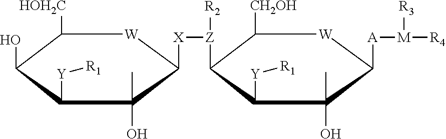

### Wherein X is O, N, S, CH2, or NH,

Wherein Z is independently a linkage to a carbohydrate composing, which could also be an oligomeric structure with a linkage consisting of O, N, S, CH2, NH, Se with a linkage R₂ as detailed below,
Wherein W is selected from the group consisting of O, N, S, CH2, NH, and Se;
Wherein Y is selected from the group consisting of O, S, C, NH, CH2, and amino acid;
Wherein R₁, R₂, R₃, and R₄ are independently selected from the group consisting of CO, SO2, SO, PO2, PO, CH, Hydrogen, and hydrophobic linear and cyclic hydrocarbons including heterocyclic substitutions of molecular weight of about 50-200 Dalton.

In some embodiments, the hydrophobic linear and cyclic hydrocarbons can comprise one of: a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, or a substituted imino group.

In some embodiments, the compound has the genera formula (2) and is for example a sulfonamide, wherein Y—R1 is triazole-3-fluorobenzene as depicted below

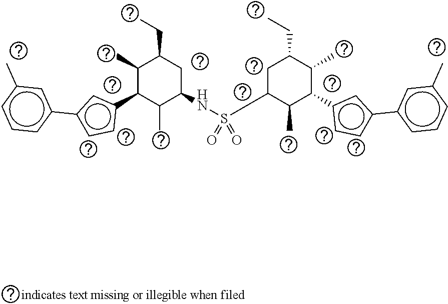

In some embodiments, the compound has the general formulas below

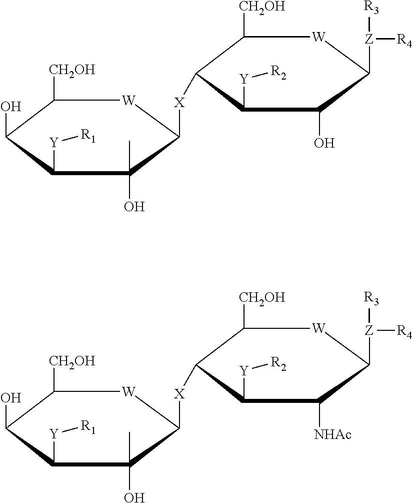

Wherein X is an amide, Sulfon, or a carbon and/or includes an aryl derivative like AM-Benzene-AM structure;
Wherein W is selected from the group consisting of O, N, S, CH2, NH, and Se; Y and Z are selected from the group consisting of O, S, C, NH, CH2, NR, Se, Amino acid and NR⁵;
Wherein R1, R2, R3, and R4 are independently selected from the group consisting of CO, SO2, SO, PO2, PO, CH, Hydrogen, Hydrophobic linear and cyclic including Heterocyclic substitutions of molecular weight of 50-200 D including, but not limited to:
a) an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted With an amino group, an alkyl group of at least 4 carbons substituted with both an amino and a carboxy group, an alkenyl group of at least 4 carbons substituted with both an amino and a carboxy group, and an alkyl group substituted with one or more halogens;
b) a phenyl group substituted with at least one car boxy group, a phenyl group substituted With at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group; and
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group.
e) saccharide; a substituted saccharide; D-galactose; substituted D-galactose; C3-[1,2,3]-triaZol-1-yl-substituted D-galactose; hydrogen, an alkyl group, an alkenyl group, an aryl group, a heteroaryl group, and a heterocycle and derivatives; an amino group, a substituted amino group, amino group, or a substituted imino group.
NR5 is selected from the group consisting of hydrogen, an alkyl group, an alkenyl group, an aryl group, a heteroaryl group, and a heterocycle. As used herein, the term “alkyl group” relates to an alkyl group containing 1-7 carbon atoms, which may include one or more unsaturated carbon atoms. In some embodiments the alkyl group contains 1-4 carbon atoms, which may include one or more unsaturated carbon atoms. The carbon atoms in the alkyl group may form a straight or branched chain. The carbon atoms in said alkyl group may also form a cycle containing 3, 4, 5, 6, or 7 carbon atoms. Thus, the term “alkyl group” used herein encompasses methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

In some embodiments, the compound is an inhibitor of Galectin-3. Without being bound to the theory, the galactose-Amide or sulfur based linker compounds described herein have an enhanced stability as its structure is less prone to hydrolysis (metabolism) and oxidation e.g. aromatic ring without substitutions, Carbon-Oxygen systems, Carbone-Nitrogen system etc.

Other aspects of the invention relate to methods for the treatment of metabolic disorders associated in part with systemic insulin resistance comprising administering to a subject in need thereof a therapeutically effective amount of a compound of general Formula (3) or a pharmaceutically acceptable salt or solvate thereof

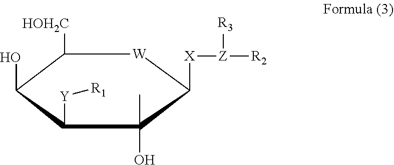

wherein X is S, O, or S(O2),
wherein W is selected from the group consisting of O, N, S, CH2, NH, and Se,
wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms,
wherein R₁, R₂, and R₃ are independently selected from the group consisting of CO, O2, SO2, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide; a substituted saccharide, D-galactose, substituted D-galactose, C3-[1,2,3]-triazol-1-yl-substituted D-galactose, hydrogen, an alkyl group, an alkenyl group, an aryl group, a heteroaryl group, and a heterocycle and derivatives, an amino group, a substituted amino group, an imino group, and a substituted imino group.

Other aspects of the invention relate to methods for the treatment of metabolic disorders associated in part with systemic insulin resistance comprising administering to a subject in need thereof a therapeutically effective amount of a compound of Formula (4) or a pharmaceutically acceptable salt or solvate thereof

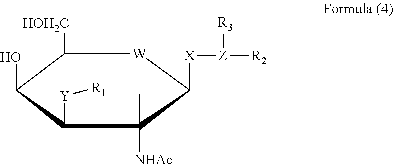

wherein X is S, O, or S(O2),

wherein W is selected from the group consisting of O, N, S, CH2, NH, and Se,

wherein Y is selected from the group consisting of O, S, NH, CH2, Se, S, S(O2), P(O2), amino acid, hydrophobic linear and cyclic hydrophobic hydrocarbons derivatives including heterocyclic substitutions of molecular weight of about 50-200 D and combinations thereof,

wherein Z is selected from the group consisting of O, S, NH, CH2, Se, P(O2), and hydrophobic hydrocarbons derivatives including heterocyclic substitutions of 3 or more atoms,

wherein R₁, R₂, and R₃ are independently selected from the group consisting of CO, O2, SO2, PO2, PO, CH, Hydrogen, combination of these and, a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives; an amino group, a substituted amino group, an imino group, and a substituted imino group.

Other aspects of the invention relate to methods for the treatment of metabolic disorders associated in part with systemic insulin resistance comprising administering to a subject in need thereof a therapeutically effective amount of a compound of general Formula (5) or a pharmaceutically acceptable salt or solvate thereof

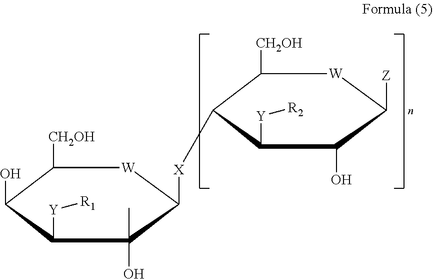

wherein X is S, O, or S(O2),
wherein W is selected from the group consisting of O, N, S, CH2, NH, and Se,
wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms,
wherein n≤24,
wherein R₁ and R₂ are independently selected from the group consisting of CO, O2, SO2, SO, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one car boxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, or a substituted imino group.

Other aspects of the invention relate to methods for the treatment of metabolic disorders associated in part with systemic insulin resistance comprising administering to a subject in need thereof a therapeutically effective amount of a compound of general Formula (6) or a pharmaceutically acceptable salt or solvate thereof

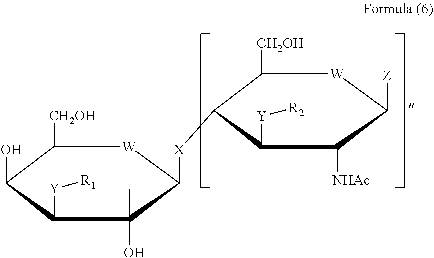

wherein X is S, O, or S(O2),
wherein W is selected from the group consisting of O, N, S, CH2, NH, and Se,
wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms,
wherein n≤24,
wherein R₁ and R₂ are independently selected from the group consisting of CO, O2, SO2, SO, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one car boxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, and a substituted imino group. In some embodiments, n=1. In other embodiments, n=2, yet in other embodiments, n=3.

Other aspects of the invention relate to methods for the treatment of metabolic disorders associated in part with systemic insulin resistance comprising administering to a subject in need thereof a therapeutically effective amount of a compound of general Formula (7) or a pharmaceutically acceptable salt or solvate thereof

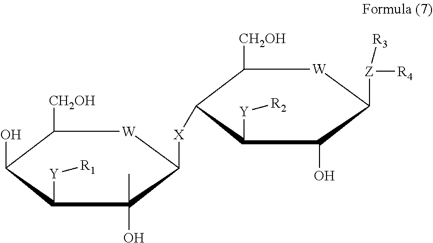

wherein X is S, O, S(O2), S—S, S—S(O2), S(O2)-S, O—S, S—O, O—S(O2), S(O2)-O, O—N(H), O—C(H2), O—C(O), O—C(H,OH), S—N(H), S—C(H2), S(O)—N(H), S(O2)-N(H), or O—P(O2),

wherein W is selected from the group consisting of O, N, S, CH2, NH, and Se,

wherein Y is selected from the group consisting of O, S, C, NH, CH2, Se, P, amino acid, an hydrophobic linear and cyclic hydrophobic hydrocarbons derivatives including heterocyclic substitutions of molecular weight of about 50-200 D and combinations thereof,

wherein Z is selected from the group consisting of O, S, N, CH, Se, S, P, and hydrophobic hydrocarbons derivatives including heterocyclic substitutions of 3 or more atoms,

wherein R₁, R₂, R₃ and R₄ are independently selected from the group consisting of CO, O2, SO2, SO, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triazol-1-yl-substituted D-galactose, hydrogen, an alkyl group, an alkenyl group, an aryl group, a heteroaryl group, and a heterocycle and derivatives, an amino group, a substituted amino group, an imino group, and a substituted imino group.

Other aspects of the invention relate to methods for the treatment of metabolic disorders associated in part with systemic insulin resistance comprising administering to a subject in need thereof a therapeutically effective amount of a compound of general Formula (8) or a pharmaceutically acceptable salt or solvate thereof

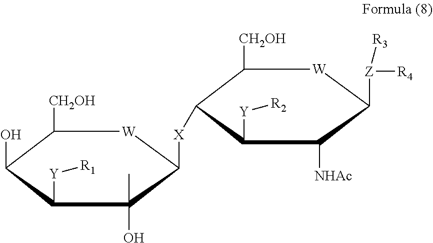

wherein X is S, O, S(O2), S—S, S—S(O2), S(O2)-S, O—S, S—O, O—S(O2), S(O2)-O, O—N(H), O—C(H2), O—C(O), O—C(H,OH), S—N(H), S—C(H2), S(O)—N(H), S(O2)-N(H), or O—P(O2),

wherein W is selected from the group consisting of O, N, S, CH2, NH, and Se,

wherein Y is selected from the group consisting of O, S, C, NH, CH2, Se, amino acid an combinations thereof,

wherein Z is selected from the group consisting of O, S, N, CH, Se, S, P, and hydrophobic hydrocarbons derivatives including heterocyclic substitutions of 3 or more atoms,

wherein R₁, R₂, R₃ and R₄ are independently selected from the group consisting of CO, O2, SO2, SO, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide; a substituted saccharide; D-galactose; substituted D-galactose, C3-[1,2,3]-triazol-1-yl-substituted D-galactose, hydrogen, an alkyl group, an alkenyl group, an aryl group, a heteroaryl group, and a heterocycle and derivatives, an amino group, a substituted amino group, an imino group, and a substituted imino group.

In some embodiments, the halogen is a fluoro, a chloro, a bromo or an iodo group.

### Synthetic Route

The compounds of this invention may be prepared by the following general methods and procedures. It should be appreciated that where typical or preferred process conditions (e.g. reaction temperatures, times, molar ratios of reactants, solvents, pressures, pH etc) are given, other process conditions may also be used unless otherwise stated. Optimum reaction conditions may vary with the particular reactants, solvents used and pH etc., but such conditions can be determined by one skilled in the art by routine optimization procedures.

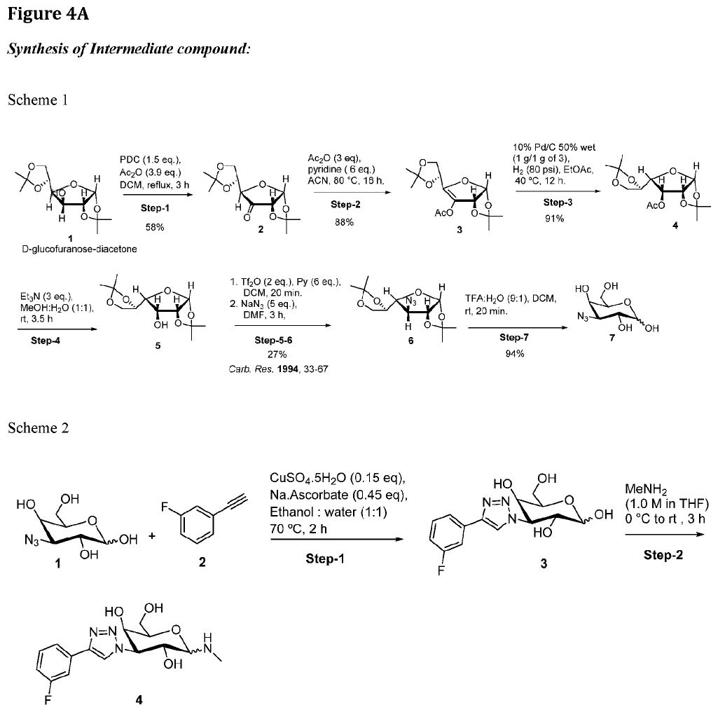

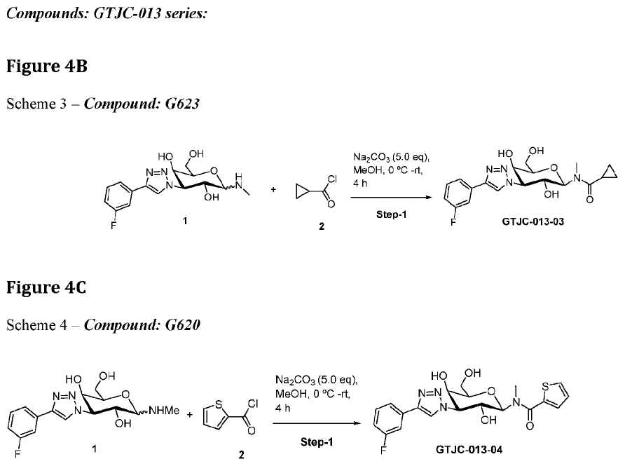

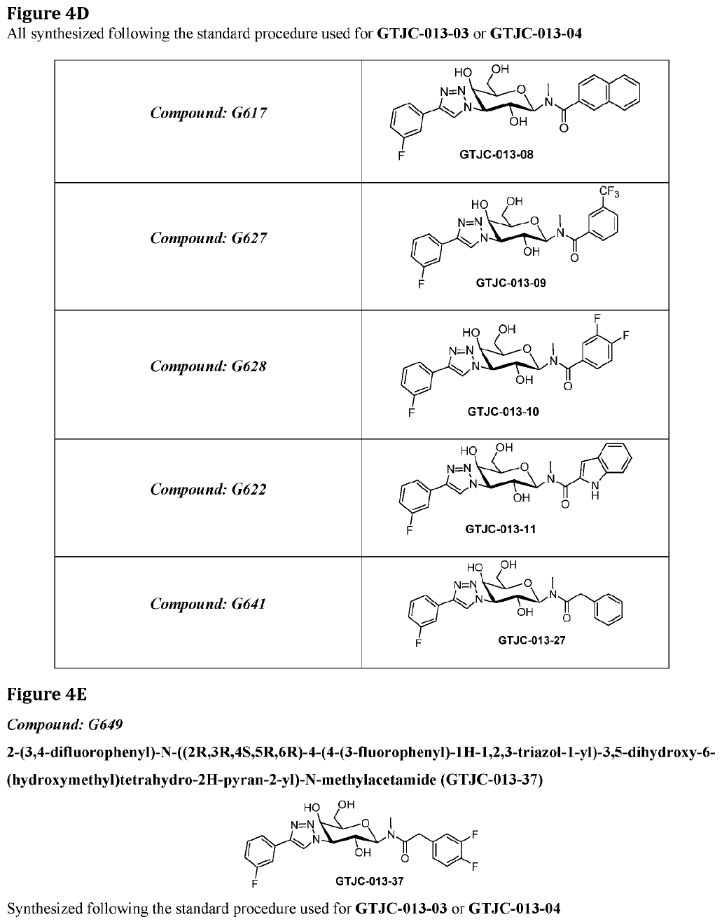

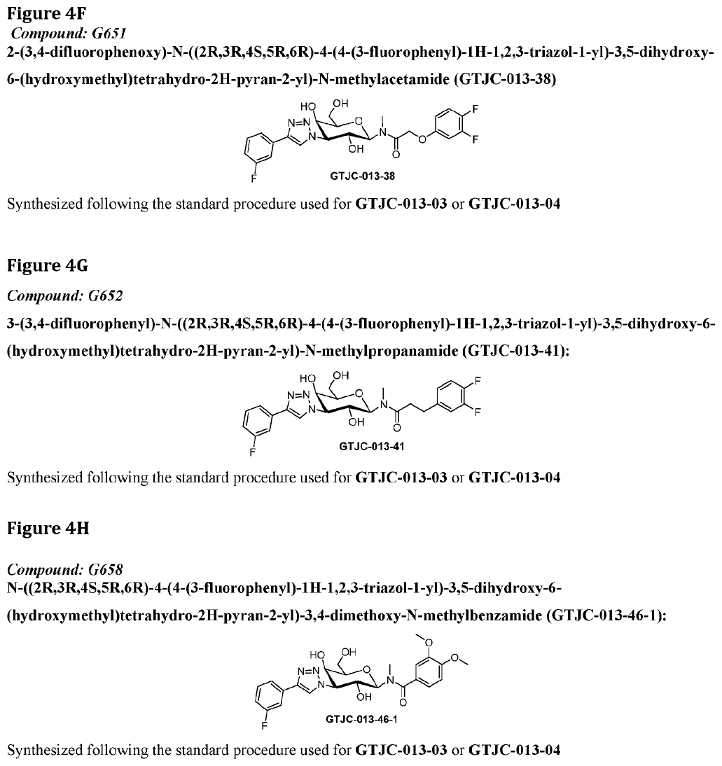

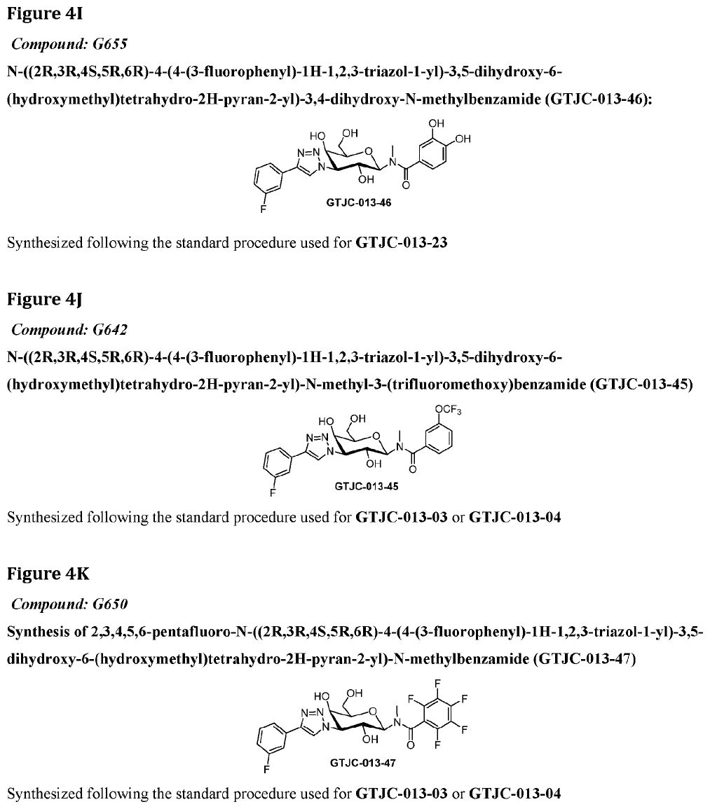

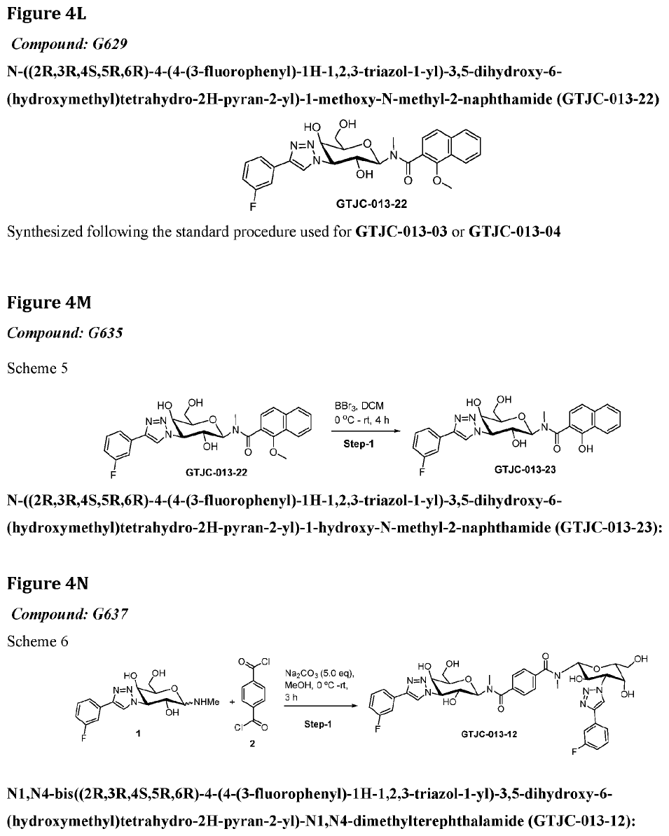

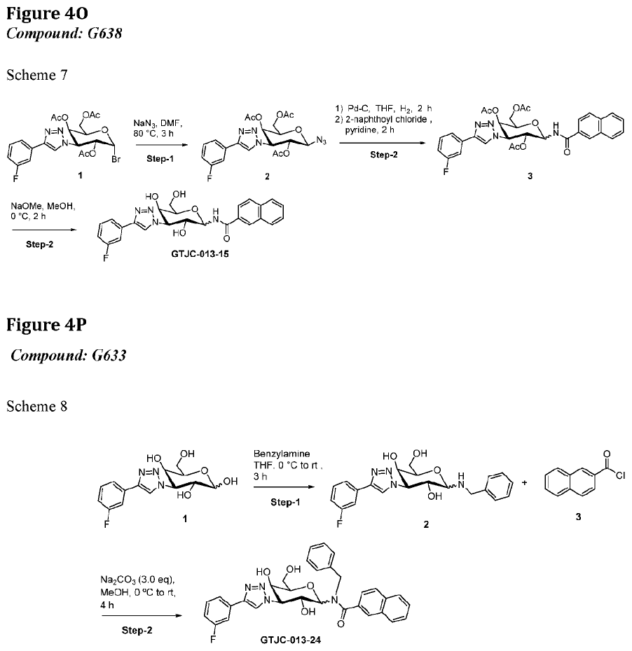

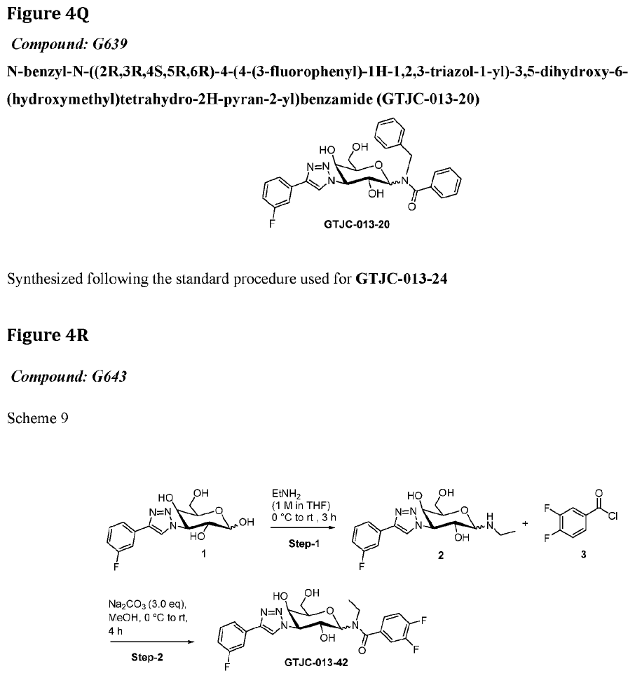

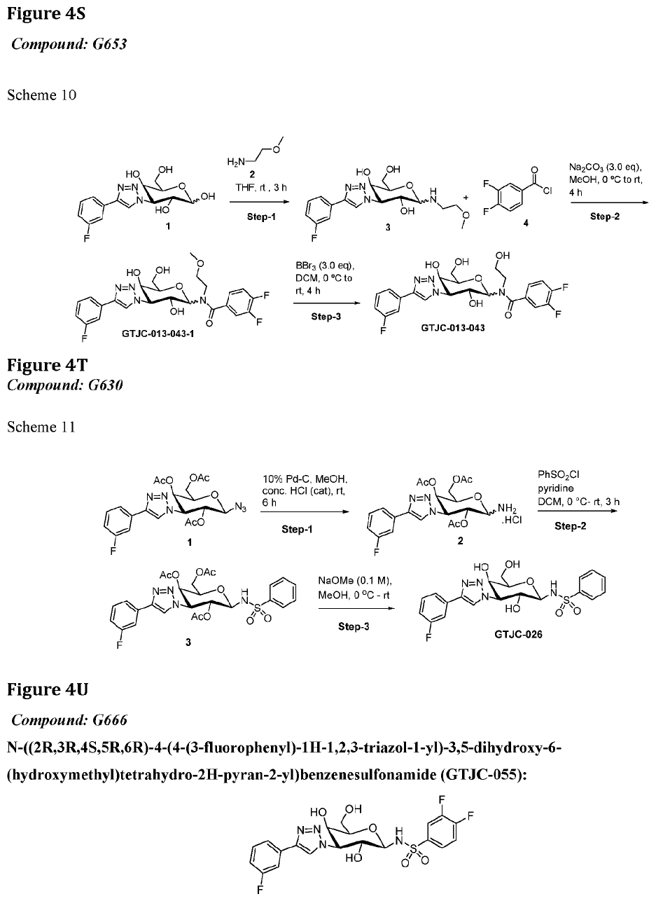

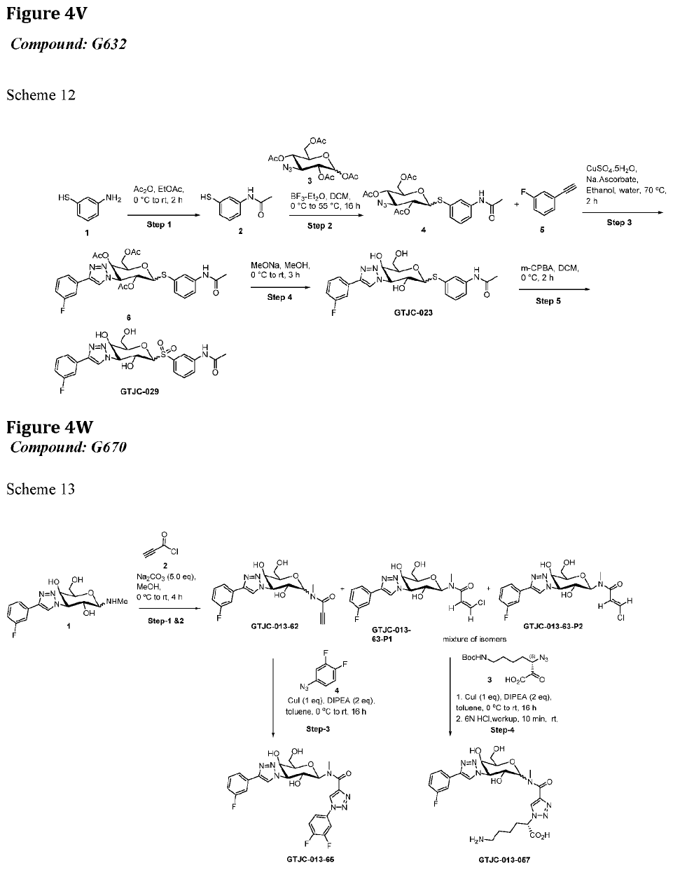

In some embodiments, the compounds were synthesized using the synthetic routes as given in Example 14 and shown in FIG. 4.

For example, compound G631 (a Galactosulfonamide, GTJC-026)

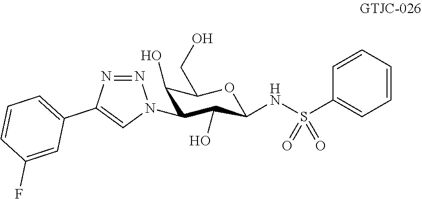

was prepared as shown in Example 14 Scheme 11 (as shown FIG. 4).

In some embodiments, the di-galactoamide compounds were synthesized using the synthetic routes as given in Example 14 Scheme 6 and shown in FIG. 4.

For example, compound G637 (a di-galactoamide with arylamide linkage, GTJC-013-12) was prepared as shown in Example 14 Scheme 6 and shown in FIG. 4.

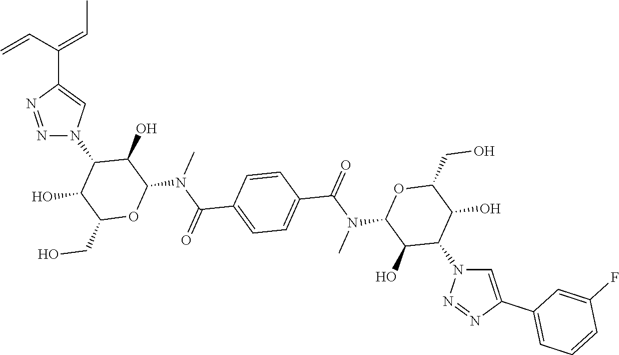 [SMILES: C=C/C(=C\C)c1cn([C@@H]2[C@@H](O)[C@H](N(C)C(=O)c3ccc(C(=O)N(C)[C@@H]4O[C@H](CO)[C@H](O)[C@H](n5cc(-c6cccc(F)c6)nn5)[C@H]4O)cc3)O[C@H](CO)[C@@H]2O)nn1]

In some embodiments, the compound was synthesized using the synthetic routes shown in FIG. 4, For example, compound G666 was prepared as shown in FIG. 4, Scheme 11.

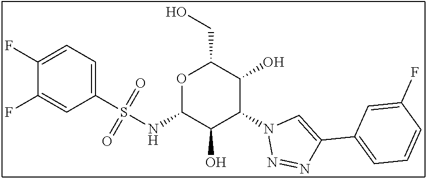 [SMILES: O=S(=O)(N[C@@H]1O[C@H](CO)[C@H](O)[C@H](n2cc(-c3cccc(F)c3)nn2)[C@H]1O)c1ccc(F)c(F)c1]

In some embodiments, the compounds were synthesized using the synthetic routes as given in Example 14 and shown in FIG. 4.

### Pharmaceutical Compositions

Aspects of the invention relate to the use of the compounds described herein for the manufacture of medicaments.

Aspects of the invention relate to pharmaceutical compositions comprising one or more of the compounds described herein. In some embodiments, the pharmaceutical compositions comprise one or more of the following: pharmaceutically acceptable adjuvant, diluent, excipient, and carrier.

The term “pharmaceutically acceptable carrier” refers to a carrier or adjuvant that may be administered to a subject (e.g., a patient), together with a compound of this invention, and which does not destroy the pharmacological activity thereof and is nontoxic when administered in doses sufficient to deliver a therapeutic amount or an effective mount of the compound.

“Pharmaceutically acceptable carrier” refers to any and all solvents, dispersion media. The use of such media and compounds for pharmaceutically active substances is well known in the art. Preferably, the carrier is suitable for oral, intravenous, intramuscular, subcutaneous, parenteral, spinal or epidural administration (e.g., by injection or infusion). Depending on the route of administration, the active compound can be coated in a material to protect the compound from the action of acids and other natural conditions that can inactivate the compound.

Some embodiments relate to the compounds or the use of the compounds having Formula (1) or Formula (2) or a pharmaceutically acceptable salt or solvate thereof. Some embodiments relate to the compounds or the use of the compounds of Table 1 or a pharmaceutically acceptable salt or solvate thereof.

Some aspects of the present invention relate to a pharmaceutical composition comprising the compound of the invention and optionally a pharmaceutically acceptable additive, such as carrier or excipient. In some embodiments, the pharmaceutical composition comprising the compound of Formula (1) or Formula (2) or a pharmaceutically acceptable salt or solvate thereof and optionally a pharmaceutically acceptable additive, such as carrier or excipient. In some embodiments, the pharmaceutical composition comprising the compound of Table 1 or a pharmaceutically acceptable salt or solvate thereof and optionally a pharmaceutically acceptable additive, such as carrier or excipient.

In some embodiments, the pharmaceutical composition comprises a compound described herein as active ingredient together with a pharmaceutically acceptable adjuvant, diluent, excipient or carrier. A pharmaceutical composition can comprise from 1 to 99 weight % of a pharmaceutically acceptable adjuvant, diluent, excipient or carrier and from 1 to 99 weight % of a compound described herein.

The adjuvants, diluents, excipients and/or carriers that may be used in the composition of the invention are pharmaceutically acceptable, i.e. are compatible with the compounds and the other ingredients of the pharmaceutical composition, and not deleterious to the recipient thereof. The adjuvants, diluents, excipients and carriers that may be used in the pharmaceutical composition of the invention are well known to a person within the art.

An effective oral dose of the compound of the present invention to an experimental animal or human may be formulated with a variety of excipients and additives that enhance the absorption of the compound via the stomach and small intestine.

The pharmaceutical composition of the present invention may comprise two or more compounds of the present invention. The composition may also be used together with other medicaments within the art for the treatment of related disorders.

In some embodiments, the pharmaceutical composition comprising a compound described herein may be adapted for oral, intravenous, topical, intraperitoneal, nasal, buccal, sublingual, or subcutaneous administration, or for administration via the respiratory tract in the form of, for example, an aerosol or an air-suspended fine powder, or, for administration via the eye, intra-ocularly, intravitreally or corneally.

In some embodiments, the pharmaceutical composition comprising a compound described herein may be in the form of, for example, tablets, capsules, powders, solutions for injection, solutions for spraying, ointments, transdermal patches or suppositories.

Some aspects of the present invention relate to pharmaceutical composition comprising the compound described herein or a pharmaceutically acceptable salt or solvate thereof and optionally a pharmaceutically acceptable additive, such as carrier or excipient.

An effective oral dose could be 10 times and up to 100 times the amount of the effective parental dose.

An effective oral dose may be given daily, in one or divided doses or twice, three times weekly, or monthly.

In some embodiments, the compounds described herein can be co-administered with one or more other therapeutic agents. In certain embodiments, the additional agents may be administered separately, as part of a multiple dose regimen, from the compounds of this invention (e.g., sequentially, e.g., on different overlapping schedules with the administration of the compound of the invention. In other embodiments, these agents may be part of a single dosage form, mixed together with the compounds of this invention in a single composition. In still another embodiment, these agents can be given as a separate dose that is administered at about the same time that the compound of the invention. When the compositions include a combination of the compound of this invention and one or more additional therapeutic or prophylactic agents, both the compound and the additional agent can be present at dosage levels of between about 1 to 100%, and more preferably between about 5 to 95% of the dosage normally administered in a monotherapy regimen.

In some embodiments, administration of the compound of the present invention and the active agent produces a synergistic effect. In some embodiments, the active agent is an antidiabetic drug. As used herein, the term “synergistic effect” refers to the correlated action of two or more agents of the present invention so that the combined action is greater than the sum of each acting separately. In some embodiments, the compounds of the present invention and the active agent can be administered simultaneously or sequentially.

Aspects of the invention relates to a composition or a compound to treat neoplastic conditions in combination with other anti-neoplastic drugs including but not limited to checkpoint inhibitors (anti-CTLA2, anti-PD1, anti-PDL1), other immune modifiers including but not limited to anti-OX40, and multiple other anti-neoplastic agents of multiple mechanisms.

In some embodiments, a therapeutically effective amount of the compound or of the composition can be compatible and effective in combination with a therapeutically effective amount of various anti-inflammatory drugs, vitamins, other pharmaceuticals and nutraceuticals drugs or supplement, or combinations thereof without limitation.

Aspects of the invention relates to a composition or a compound to treat neoplastic conditions in combination with other anti-neoplastic drugs including but not limited to checkpoint inhibitors (anti-CTLA2, anti-PD1, anti-PDL1), other immune modifiers including but not limited to anti-OX40, and multiple other anti-neoplastic agents of multiple mechanisms.

### Methods of Treatment

Some aspects of the invention relate to the use of the compounds described herein or the composition described herein for us in the treatment of a disorder relating to the binding of a Galectin to a ligand. In some embodiments, Galectin is Galectin-3.

Some aspects of the invention relate to the method of treating various disorders relating to the binding of a Galectin to a ligand. In some embodiments, the methods comprise administering in a subject in need thereof a therapeutically effective amount of at least one compound described herein. In some embodiments, the subject in need thereof is a human having high levels of Galectin-3. Levels of Galectin, for example Galectin-3 can be quantified using any methods known in the art.

Some aspects of the invention relate to a method of treating diseases due to disruption in the activity of TGFb1 (Transforming Growth Factor beta 1) by reversal of the Galectin-3 interaction with its receptor (TGFb1-Receptor) so as to recover normal regenerative activity in tissues.

Some aspects of the invention relate to a method of treating diseases associated with the Transforming Growth Factor Beta signaling pathway that involved many cellular and pathological processes in both the adult and embryo development including cell growth, cell differentiation, apoptosis, cellular homeostasis and other cellular functions.

Some aspects of the present invention relate to a method for treatment of a disorder relating to the binding of a Galectin, such as Galectin-3 binding to an Insulin-Receptor or TGFb1-receptor in a human, wherein the method comprises administering a therapeutically effective amount of at least one compound of Formula (1) or Formula (2), Table 1, or a pharmaceutically acceptable salt or solvate thereof to a human in need thereof.

Aspects of the invention relate to compounds, compositions and methods for the treatment of, but not limited to, systemic insulin resistance. In some embodiments, the systemic insulin resistance is associated with obesity where elevated galectin-3 interacts with insulin receptor. In some embodiments, treatment with compounds of this invention can restore sensitivity to insulin activity in various tissues.

Aspects of the invention relate to compounds, compositions and methods for the treatment of systemic insulin resistance associated with type 1 diabetes. Aspects of the invention relate to compounds, compositions and methods for the treatment of systemic insulin resistance associated with type 2 diabetes mellitus (T2DM). Aspects of the invention relate to compounds, compositions and methods for the treatment of systemic insulin resistance associated with obesity, gestational diabetes and prediabetes. In some embodiments, the compound restores sensitivity of cells to insulin activity. In some embodiments, the compound inhibits galectin-3 interaction with Insulin receptor, which interferes with insulin binding and cellular glucose uptake mechanism. Aspects of the invention relate to compounds, compositions and methods for the treatment of low-grade inflammation, due to elevated levels of free fatty acid and triglycerides that cause insulin resistance in skeletal muscle and liver which contributes to the development of atherosclerotic vascular diseases and NAFLD. Aspects of the invention relate to compounds, compositions and methods for the treatment of polycystic ovarian syndrome (PCOS) associated with obesity, insulin resistance, and the compensatory hyperinsulinemia which affects some 65-70% of women with PCOS. Aspects of the invention relate to compounds, compositions and methods for the treatment of diabetic nephropathy and glomerulosclerosis by attenuating integrin and TGFβ Receptor pathway in kidney chronic disease. In some embodiments, the compound can inhibit the overexpression of TGFβ receptor signaling system triggered by Insulin resistance in diabetic and cause decline in renal function, and can reverse the established lesions of diabetic glomerulopathy.

In some embodiments, the compound is administered with a pharmaceutically acceptable adjuvant, excipient, formulation carrier or combinations thereof. In some embodiments, the compound is administered with an active agent and a pharmaceutically acceptable adjuvant, excipient, formulation carrier or combinations thereof. In some embodiments, the compound is administered with one or more anti diabetic drug. In some embodiments, administration of the compound of the present invention and the active agent produces a synergistic effect.

Aspects of the invention relate to compounds, compositions and methods of treating systemic insulin resistance associated with obesity where elevated galectin-3 interacts with insulin receptor. In some embodiments, treatment with compounds of this invention can restore sensitivity to insulin activity in various tissues.

In some embodiments, the compounds or compositions of the invention that bind to insulin receptor (also identified as IR, INSR, CD220, HHF5).

Aspects of the invention relate to compounds, compositions and methods of treating diseases caused by disruption in the activity of TGFb1 (Transforming Growth Factor beta 1).

In some embodiments, the disorder is an inflammatory disorder, for example inflammatory bowel disease, Crohn's disease, multiple sclerosis, systemic lupus erythematosus, or ulcerative colitis.

In some embodiments, the disorder is fibrosis, for example liver fibrosis, pulmonary fibrosis, kidney fibrosis, heart fibrosis or fibrosis of any organ compromising the normal function of the organ.

In some embodiments, the disorder is cancer.

In some embodiments, the disorder is an autoimmune disease such as rheumatoid arthritis and multiple sclerosis.

In some embodiments, the disorder is heart disease or heart failure.

In some embodiments, the disorder is a metabolic disorder, for example diabetes.

In some embodiments, the disorder relating is pathological angiogenesis, such as ocular angiogenesis, disease or conditions associated with ocular angiogenesis and cancer.

In some embodiments, the composition or the compound can be used in the treatment of nonalcoholic steatohepatitis with or without liver fibrosis, inflammatory and autoimmune disorders, neoplastic conditions or cancers.

In some embodiments, the composition can be used in the treatment of liver fibrosis, kidney fibrosis, lung fibrosis, or heart fibrosis.

In some embodiments, the composition or the compound is capable of enhancing anti-fibrosis activity in organs, including but not limited to, liver, kidney, lung, and heart.

In some embodiments, the composition or the compound can be used in treatment of inflammatory disorders of the vasculature including atherosclerosis and pulmonary hypertension.

In some embodiments, the composition or the compound can be used in the treatment of heart disorders including heart failure, arrhythmias, and uremic cardiomyopathy.

In some embodiments, the composition or the compound can be used in the treatment of kidney diseases including glomerulopathies and interstitial nephritis.

In some embodiments, the composition or the compound can be used in the treatment of inflammatory, proliferative and fibrotic skin disorders including but not limited to psoriasis and scleroderma.

Aspects of the invention relates to methods of treating allergic or atopic conditions, including but not limited to eczema, atopic dermatitis, or asthma.

Aspects of the invention relates to methods of treating inflammatory and fibrotic disorders in which galectins are at least in part involved in the pathogenesis, by enhancing anti-fibrosis activity in organs, including but not limited to liver, kidney, lung, and heart.

Aspects of the invention relates to methods relates to a composition or a compound that has a therapeutic activity to treat nonalcoholic steatohepatitis (NASH). In other aspects, the invention elates to a method to reduce the pathology and disease activity associated with nonalcoholic steatohepatitis (NASH).

Aspects of the invention relates to a composition or a compound used in treating or a method of treating inflammatory and autoimmune disorders in which galectins are at least in part involved in the pathogenesis including but not limited to arthritis, systemic lupus erythematosus, rheumatoid arthritis, asthma, and inflammatory bowel disease.

Aspects of the invention relates to a composition or a compound to treat neoplastic conditions (e.g. benign or malignant neoplastic diseases) in which galectins are at least in part involved in the pathogenesis by inhibiting processes promoted by the increase in galectins. In some embodiments, the invention relates a method of treating neoplastic conditions (e.g. benign or malignant neoplastic diseases) in which galectins are at least in part involved in the pathogenesis by inhibiting processes promoted by the increase in galectins. In some embodiments, the composition or a compound can be used to treat or prevent tumor cell growth, invasion, metastasis, and neovascularization. In some embodiments, the composition or a compound can be used to treat primary and secondary cancers.

### Examples

### Example 1: Compound Inhibition of Galectin Binding to Labeled Probes

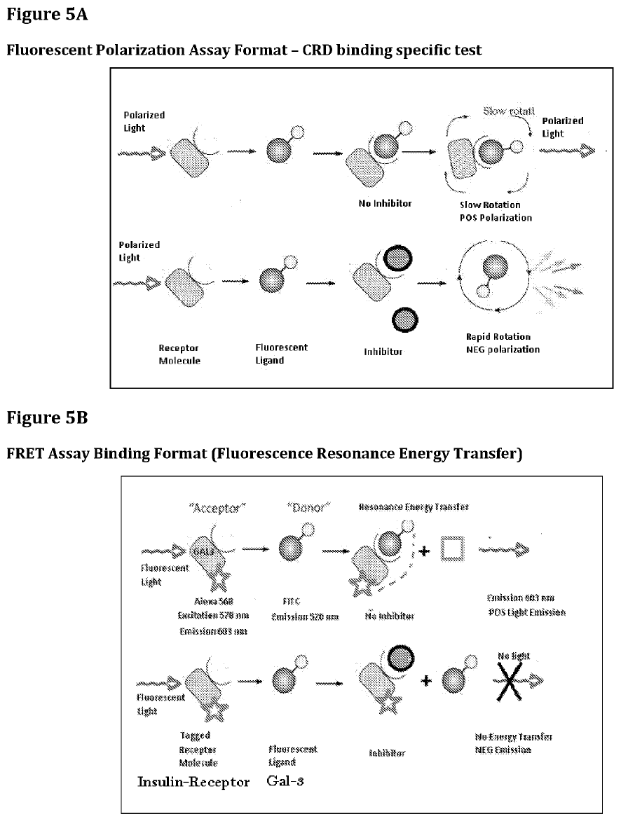

Fluorescein-labeled probes have been developed which bind to Galectin 3 and other Galectin proteins and these probes have been used to establish assays (FIGS. 5A & 5B) that measure the binding affinity of ligands for the Galectin proteins using Fluorescence Polarization (Sorme P, et al. Anal Biochem. 2004 Nov. 1; 334(1):36-47).

Compounds described herein avidly bind to Galectin-3, as well as other Galectin proteins, using this assay format (FIG. 5A) and displace the Fluorescein-labeled probe with high affinity, with IC₅₀'s (concentration at 50% inhibition) of between about 5 ηM to about 40 μM. In some embodiments, the IC50 is about from 5 nM to about 20 nM. In some embodiments, the IC50 is from about 5 nM to about 100 nM. In some embodiments, the IC50 is from about 10 nM to about 100 nM. In some embodiments, the IC50 is from about 50 nM to about 5 μM. In some embodiments, the IC50 is from about 0.5 μM to about 10 μM. In some embodiments, the IC50 is from about 5 μM to about 40 μM.

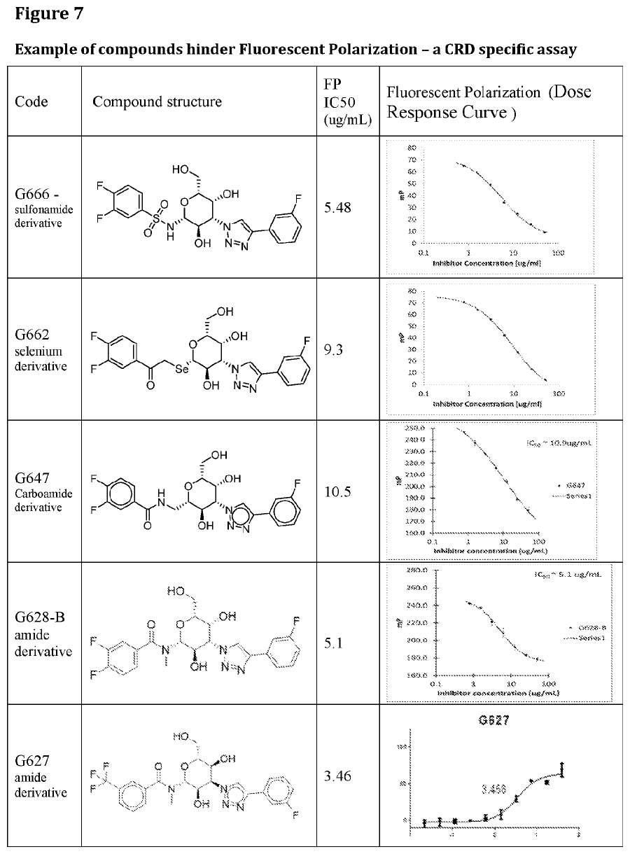

Compounds claimed under this invention were synthesized (FIG. 4) and showed an inhibitory activity in the Fluorescent polarization assay (FIG. 7).

### Example 2: Compound Inhibition of Galectin Binding Using FRET Assay

FRET assay (fluorescent resonance energy transfer) assays were developed for evaluating the interaction of Galectin proteins, including but not limited to Galectin-3, with a model fluorescent-labeled probe (see FIG. 5B). Using this assay, compounds described herein avidly bind to Galectin-3, as well as other Galectin proteins, using this assay and displace the probe with high affinity, with IC₅₀'s (concentration at 50% inhibition) of between about 5 ηM to about 40 μM. In some embodiments, the IC50 is about from 5 nM to about 20 nM. In some embodiments, the IC50 is from about 5 nM to about 100 nM. In some embodiments, the IC50 is from about 10 nM to about 100 nM. In some embodiments, the IC50 is from about 50 nM to about 5 μM. In some embodiments, the IC50 is from about 0.5 μM to about 10 μM. In some embodiments, the IC50 is from about 5 μM to about 40 μM.

### Example 3: Compound Inhibition of Galectin Binding to Physiologic Ligands

Galectin proteins, including but not limited to Galectin-3 and Galectin-1, have multiple biologically relevant binding ligands in mammalian species, including but not limited to rodents, primates, and humans. Galectins are carbohydrate-binding proteins that bind to glycoproteins with β-galactoside-containing sugars. The result of binding of Galectin proteins to these ligands results in a plethora of biological effects in and on cells and in tissues and whole organisms including regulating cell survival and signaling, influencing cell growth and chemotaxis, interfering with cytokine secretion, mediating cell-cell and cell-matrix interactions or influencing tumor progression and metastasis. Additionally, changes in normal expression of Galectin proteins are responsible for pathological effects in multiple diseases, including but not limited to inflammatory, fibrotic and neoplastic diseases.

Compounds described in this invention are designed to bind to the carbohydrate recognition domain of Galectin proteins, including but not limited to Galectin-3, and disrupt interactions with biologically relevant ligands. They are intended to inhibit the function of Galectin proteins that may be involved in pathological processes at normal levels of expression or in situations where they are increased over physiological levels.

Some of the ligands for Galectin proteins that are important in normal cellular function and pathology in disease include, but are not limited to, TIM-3 (T cell immunoglobulin mucin-3)), CD8, T cell receptor, integrins, Galectin-3 binding protein, TGF-β receptor, Insulin Receptor, laminins, fibronectins, BCR (B cell receptor, CTLA-4 (cytotoxic T-lymphocyte-associated protein-4), EGFR (Epidermal growth factor receptor), FGFR (fibroblast growth factor receptor), GLUT-2 (glucose transporter-2), IGFR (insulin-like growth factor receptor), various interleukins, LPG (lipophosphoglycan), MHC (major histocompatibility complex), PDGFR (platelet-derived growth factor receptor), TCR (T cell receptor), TGF-β (transforming growth factor-β), TGFβR (transforming growth factor-β receptor, CD98, Mac3 antigen (Lysosome-associated membrane protein 2 (LAMP2) also known as CD107b (Cluster of Differentiation 107b)).

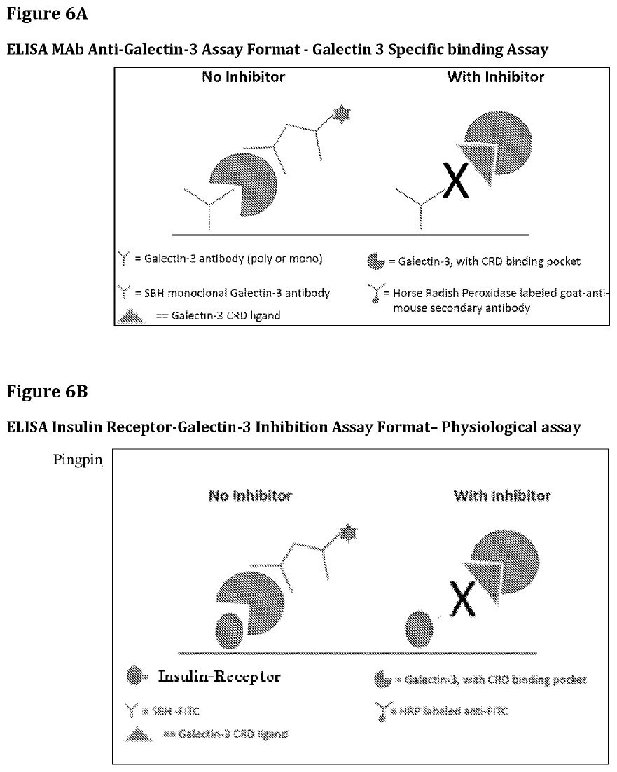

Experiments have been performed to evaluate the physical interaction of Galectin proteins with these various biological ligands mediating cellular functions. The experiments were designed to evaluate the interaction between various Galectin-3 ligands and determine whether compounds described herein are able to inhibit these interactions, as analytical assays format shown in FIGS. 6A and 6B.

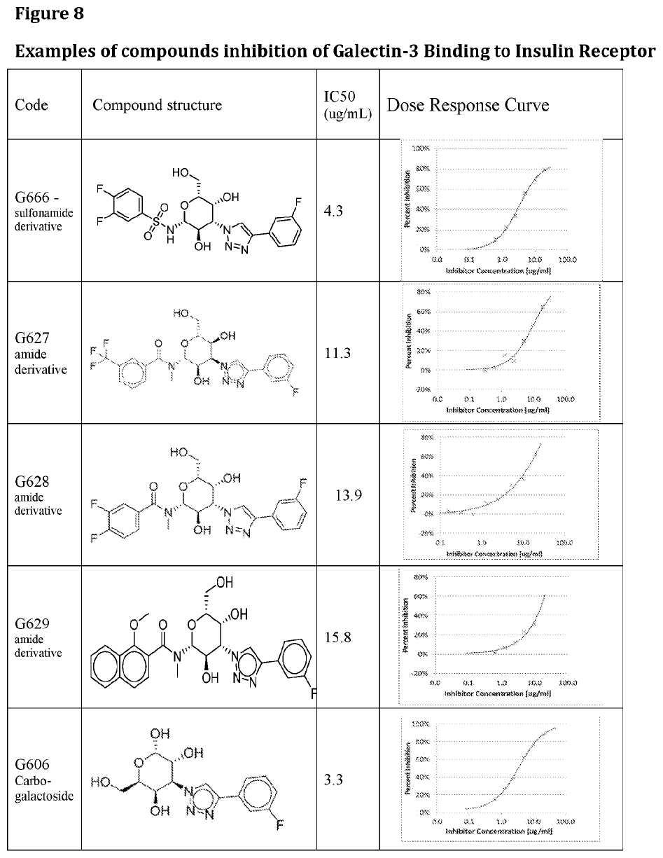

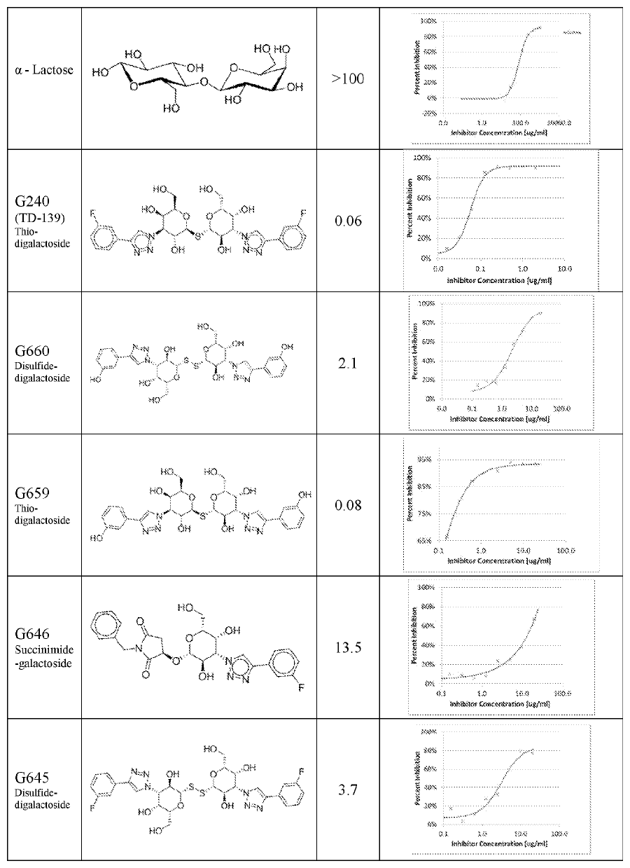

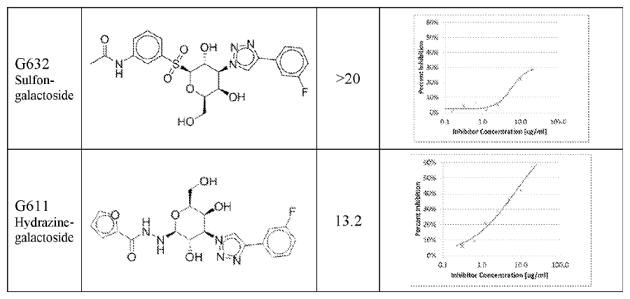

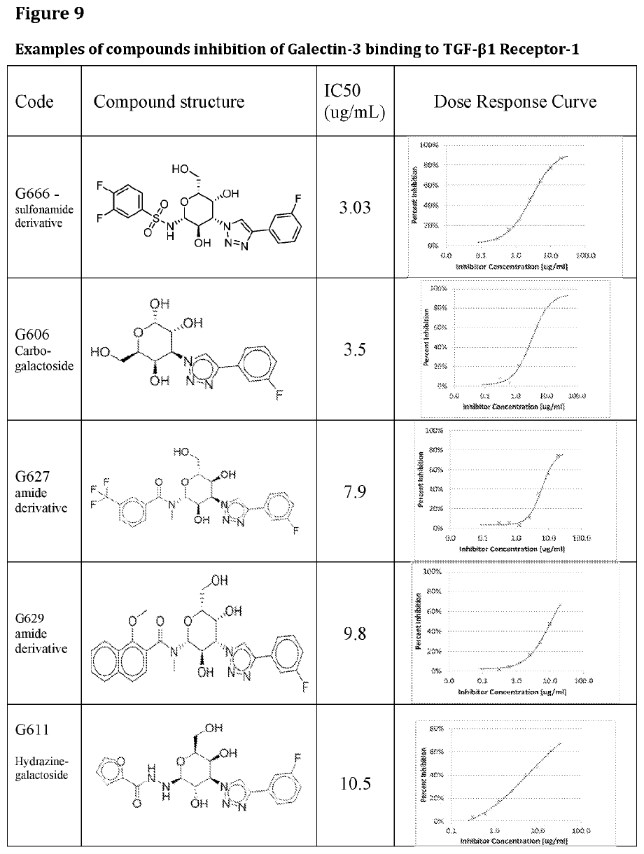

Using these assays format, the compounds described herein inhibit the interaction of Galectin-3 with Insulin Receptor and TGFb1-receptor (FIG. 8 & FIG. 9). The compounds have previously shown to inhibited Galectin proteins interaction with other ligands, including but not limited to various integrin molecules (αVβ3, αVβ6, αMβ2, α2β3, and others) with IC50's in the range of about 5 ηM to about 40 μM. In some embodiments, the IC50 is about from 5 nM to about 20 nM. In some embodiments, the IC50 is from about 5 nM to about 100 nM. In some embodiments, the IC50 is from about 10 nM to about 100 nM. In some embodiments, the IC50 is from about 50 nM to about 5 μM. In some embodiments, the IC50 is from about 0.5 μM to about 10 μM. In some embodiments, the IC50 is from about 5 μM to about 40 μM (FIGS. 8 and 9).

### Example 4: Compound Binding to Amino Acid Residues in Galectin Proteins

Heteronuclear NMR spectroscopy is used to evaluate the interaction of compounds described herein with Galectin molecules, including but not limited to Galectin-3, to assess the interaction residues on the Galectin-3 molecule.

Uniformly ¹⁵N-labeled Galectin-3 is expressed in BL21 (DE3) competent cells (Novagen), grown in minimal media, purified over a lactose affinity column, and fractionated on a gel filtration column, as described previously for production of Gal-1 (Nesmelova I V, Pang M, Baum L G, Mayo K H. 1H, 13C, and 15N backbone and side-chain chemical shift assignments for the 29 kDa human Galectin-1 protein dimer. Biomol NMR Assign 2008 December; 2(2):203-205).

Uniformly ¹⁵N-labeled Galectin-3 is dissolved at a concentration of 2 mg/ml in 20 mM potassium phosphate buffer at pH 7.0, made up using a 95% H₂O/5% D₂O mixture. ¹H-¹⁵N HSQC NMR experiments are used to investigate binding of a series of compounds described herein. ¹H and ¹⁵N resonance assignments for recombinant human Galectin-3 were previously reported (Ippel H, et al. (1)H, (13)C, and (15)N backbone and side-chain chemical shift assignments for the 36 proline-containing, full length 29 kDa human chimera-type Galectin-3. Biomol NMR Assign 2015 April; 9(1):59-63.).

NMR experiments are carried out at 30° C. on Bruker 600 MHz, 700 MHz or 850 MHz spectrometers equipped with H/C/N triple-resonance probes and x/y/z triple-axis pulse field gradient units. A gradient sensitivity-enhanced version of two-dimensional ¹H-¹⁵N HSQC is applied with 256 (t1)×2048 (t2) complex data points in nitrogen and proton dimensions, respectively. Raw data are converted and processed by using NMRPipe and were analyzed by using NMRview.

These experiments show differences between compounds described herein in the binding residues in the carbohydrate binding domain of Galectin-3.

### Example 5: Cellular Activity of Cytokine Expression Related to Galectin Binding Inhibition

Example 3 describes the ability of compounds of this application to inhibit the binding of physiologic ligands to Galectin molecules. In the experiments of this example, the functional implications of those binding interactions are evaluated.

One of the interactions with Galectin-3 that is inhibited by the compounds described herein was TGF-β receptor. Therefore, experiments are done to evaluate the effect of compounds on TGR-β receptor activity in cell lines. Various TGF-β responsive cell lines, including but not limited to LX-2 and THP-1 cells, are treated with TGF-β and response of the cells is measured by looking at activation of second messenger systems, including but not limited to phosphorylation of various intracellular SMAD proteins. After establishing that TGF-β activates the second messenger systems in the various cell lines, the cells are treated with compounds described herein. This experiments show that these compounds inhibit TGF-β signaling pathways, confirming that the binding interaction inhibition described in Example 1 has a physiological role in cellular models.

Cellular assays are also performed to evaluate the physiological significance of inhibiting the interaction of Galectin-3 with various integrin molecules. Cell-cell interaction studies are performed using monocytes binding to vascular endothelial cells, as well as other cell lines. Treatment of cells with compounds described herein is found to inhibit these integrin-dependent interactions, confirming that the binding interaction inhibition described in Example 1 has a physiological role in cellular models.

Cellular motility assays are performed to evaluate the physiological significance of inhibiting the interaction of Galectin-3 with various integrin and other cell surface molecules defined in Example 3. Cellular studies are performed using multiple cell lines in a semi-permeable membrane separated well apparatus. Treatment of cells with compounds described herein is found to inhibit cellular motility, confirming that the binding interaction inhibition described in Example 3 has a physiological role in cellular models.

### Example 6: In-Vitro Inflammatory Model (a Monocyte Based Assay)

A model of macrophage polarization is set up, starting from THP-1 monocytes culture which is differentiated into inflammatory macrophages using PMA (Phorbol 12-myristate 13-acetate) for 2-4 days. Once differentiated (M0 macrophages), the macrophages are induced with LPS or LPS and IFN-gamma for macrophage activation (M1) to inflammatory stage for 1-3 days. Array of cytokines and chemokines are analyzed to confirm the polarization of THP-1-derived macrophages to inflammatory stage. The impact of the anti-Galectin-3 compounds on macrophage polarization is assessed first by monitoring cell viability using a colorimetric method (using a tetrazolium reagent) to determine the number of viable cells in proliferation or cytotoxicity assays (Promega, The CellTiter 96® AQueous One Solution Cell Proliferation Assay which contains a novel tetrazolium compound [3-(4,5-dimethyl-2-yl)-5-(3-carboxymethoxyphenyl)-2-(4-sulfophenyl)-2H-tetrazolium, inner salt; MTS] and an electron coupling reagent (phenazine ethosulfate; PES)) and inflammatory stage evaluated by a quantitatively measure of the chemokine Monocyte Chemoattractant Protein-1 (MCP-1/CCL2), a key protein that regulates migration and infiltration of monocytes/macrophages in cellular process of inflammation. Follow-up testing for the expression and secretion of other cytokines and chemokines are done for leading active compounds. Results are expressed in percentage reduction of MCP-1.

Example of method steps:

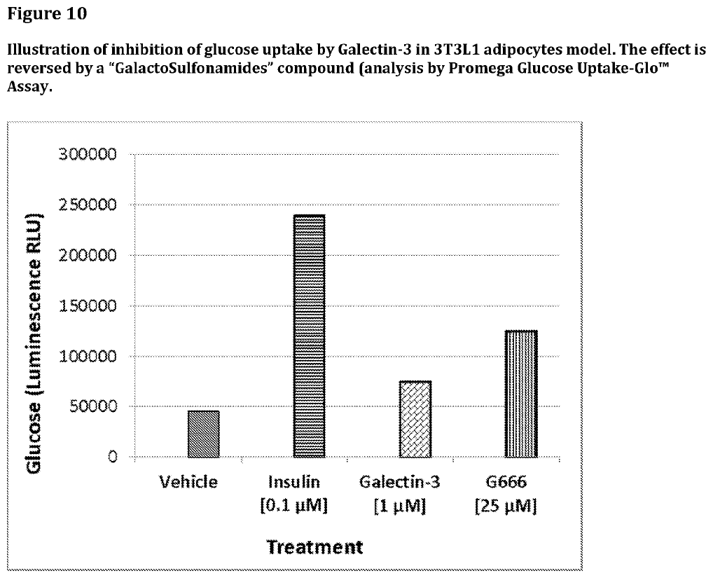

1) THP-1 cells were cultured in media containing Gentamicin
2) THP-1 cells are transfer to wells in a 96 well plate 2,000 cells/well for 2 days incubation in assay media containing 5-50 ng/ml PMA
3) Serial dilution of test compounds is made in LPS (1-10 ng/ml) containing media
4) To each well 100 ml of compounds/LPS solution is added to a final assay volume of each well of 200 ml which contains also Gentamicin and 5-20 ng/ml PMA
5) Cells are incubated up to 8 days.
6) Every other day samples of 20-60 ul are removed for biomarker assays
7) At termination 15 ml of Promega Substrate CellTiter 96 Aqueous One Solution is prepared and added to each well to monitor cytotoxicity (at 490 nm)
8) For cellular biomarkers evaluation the cells are washed 1×PBS and extracted with 200 ul of Lysis buffer for 1 hour. Extract is spinned down 10 minutes and 120 ul sample is removed from top. All samples are kept at −70 C until testing. (See FIG. 10)

### Example 7: Cell Culture Adipocyte Model

Cell Culture and Insulin Resistance model used 3T3-L1 fibroblasts that were cultured in DMEM containing 10% FCS and GlutaMAX and differentiated to adipocytes as previously reported (Shewan, A. M., van Dam, E. M., Martin, S., Luen, T. B., Hong, W., Bryant, N. J., and James, D. E. (2003) “GLUT4 recycles via a trans-Golgi network (TGN) subdomain enriched in Syntaxins 6 and 16 but not TGN38: involvement of an acidic targeting motif”. Mol. Biol. Cell 14, 973-986). Various Insulin resistance models were used. 3T3-L1 adipocytes were cultured with various doses of insulin (10 nM to 100 nM) to cause chronic insulin exposure or 0.1M to 1M dexamethasone (DEX) for 8 to 24 h at 37° C. or with 1 to 20 ng/ml TNF at 37° C. for 48 h in full DMEM medium. The medium was replaced twice a day with fresh medium containing TNF. After insulin resistance treatment, cells were washed and then serum starved for 1-2 h prior to insulin stimulation and assessment of insulin-regulated kinases and processes. It has been previously shown that this protocol is adequate to return the cells to their baseline level of GLUT4 translocation (Hoehn, K. L., Hohnen-Behrens, C., Cederberg, A., Wu, L. E., Turner, N., Yuasa, T., Ebina, Y., and James, D. E. (2008) IRS1-independent defects define major nodes of insulin resistance. Cell Metab. 7, 421-433)

Experiments were performed with 3T3-L1 fibroblasts differentiated to adipocytes cultures following the Promega protocol:
1. On Day 1, 1 ml vial of low passage number 3T3L1 cells was thawed and combined with 9 ml of Maintenance Medium (MM). The cells were centrifuged at 200×g for 10 minutes and the liquid medium was aspirated.
2. The cell pellet was resuspended in 11 ml of MM. The cells were plated at 20,000 cells per 100 μl in a 96-well plate.
3. The cells were grown to confluency at 37° C. in 5% CO2 with medium replacement every 2 days. Because of the weak adherence of these cells during differentiation, cells were plated on collagen coated plates (Corning, Cat.#356650). Medium removal and addition was performed at the slowest pipetting speeds possible.
4. On Day 5, the medium was replaced with 100 μl Differentiation Medium I (DM-I) and continued to be replaces every 2 days.
5. On Day 12, the medium was replaced with 100 μl Differentiation Medium II (DM-II).
6. On Day 14, the medium was replaced with 100 μl of MM. the medium was continued to be replaced every 2 days.
7. Insulin responses were measured between 8-11 days.

3T3L1 adipocytes were assayed as follows:
1. The medium was replaced with 100 μl MM without serum one day before the assay,
2. On the day of the assay, the medium was replaced with 100p DMEM without serum or glucose (Life Technologies, Cat.#11966) containing a range of insulin concentrations. The cells were incubated for 1 hour at 37° C. in 5% CO2.
3. The medium was removed and 50 μl of 2DG (1 mM) in PBS were added and cells were incubated for 10 minutes at 25° C.
4. 25 μl of Stop Buffer was added and the sample was briefly shaken.
5. 25 μl of Neutralization Buffer was added and the sample was briefly shaked.
6. 100 μl of 2DG6P Detection Reagent was added, the sample was briefly shaked and incubated for 1 hour at 25° C.
7. Luminescence was recorded with 0.3-1 second integration on a luminometerto evaluate the cellular effect of Galectin-3 on glucose uptake.

Differentiation of adipocytes cells was monitored by various well-defined insulin related activation markers, including expression of Insulin Receptor (IR) and its activation by insulin, but not limited to IR kinase activity within minutes of exposing to insulin. Inhibition of this insulin activation by treatment with Galectin-3. The effect of Galectin-3 on IR was monitored also by rate of glucose uptake.

The compounds described herein were found to inhibit the effect of Galectin-3 and reversal of insulin resistance and recovering of glucose uptake (FIG. 10) confirming a physiological and potential therapeutic role in systemic insulin resistance in diabetes linked to obesity.

### Example 8: Evaluation of Compound Absorption, Distribution, Metabolism, and Elimination

Compounds described herein are evaluated for physicochemical properties, including but not limited to solubility (Thermodynamic and Kinetic method), various pH changes, solubility in biorelevant medium (FaSSIF, FaSSGF, FeSSIF), Log D (Octanol/water and Cyclohexane/water), chemical stability in plasma, and blood partitioning.

Compounds described herein are evaluated for in vitro permeability properties, including but not limited to PAMPA (parallel artificial membrane permeability assay), Caco-2, and MDCK (wild type)

Compounds described herein are evaluated for animal pharmacokinetic properties, including but not limited to pharmacokinetics by various routes viz., oral, intravenous, intraperitoneal, subcutaneous in mice (Swiss Albino, C57, Balb/C), rats (Wistar, Sprague Dawley), rabbits (New Zealand white), dogs (Beagle), Cynomolgus monkeys, etc., tissue distribution, brain to plasma ratio, biliary excretion, and mass balance.

Compounds described herein are evaluated for protein binding, including but not limited to plasma protein binding (ultra Filtration and Equilibrium Dialysis) and microsomal protein binding.

Compounds described herein are evaluated for in vitro metabolism, including but not limited to cytochrome P450 inhibition, cytochrome P450 time dependent inhibition, metabolic stability, liver microsome metabolism, S-9 fraction metabolism, effect on cryopreserved hepatocyte, plasma stability, and GSH trapping.

Compounds described herein are evaluated for metabolite identification, including but not limited to identification in vitro (microsomes, S9 and hepatocytes) and in vivo samples.

### Example 9: Synthesis of GalactoAmide and GalactoSulfonamides Compounds

Non-limiting examples of compounds according to some embodiments are shown in Table 1

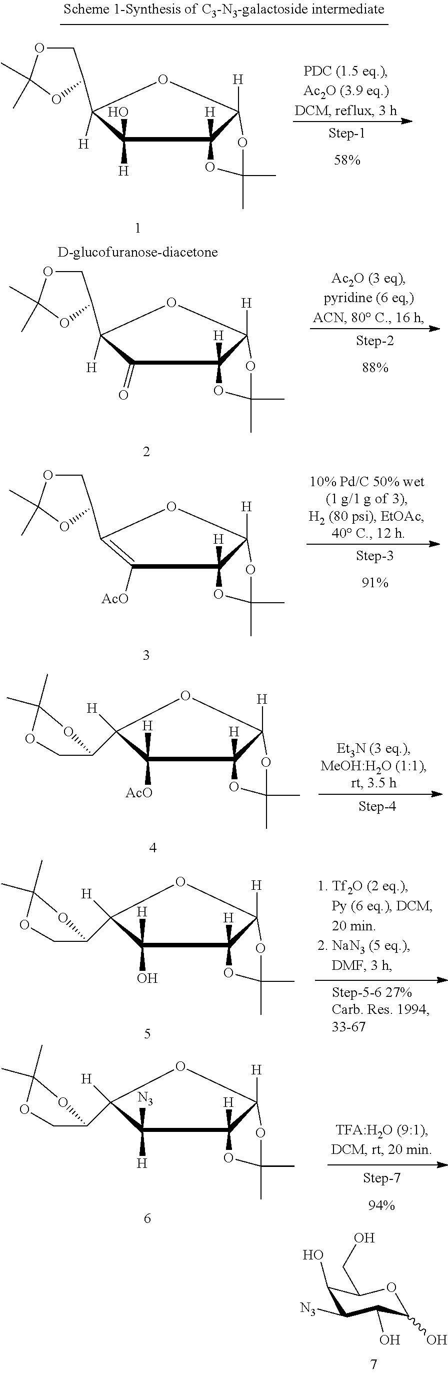

### Step-1

### (3aR,5R,6aS)-5-((S)-2,2-dimethyl-1,3-dioxolan-4-yl)-2,2-dimethyldihydrofuro [2,3-d][1,3]dioxol-6(5H)-one

To a stirred solution of (3aR,5S,6S,6aR)-5-((S)-2,2-dimethyl-1,3-dioxolan-4-yl)-2,2-dimethyltetrahydrofuro[2,3-d][1,3]dioxol-6-ol (1000 g, 3846 mmol) in DCM (8000 mL) was added Ac20 (3.9 eq) followed by PDC (1.5 eq) portion wise at room temperature over a period of 2 h. The reaction mixture was reflux for 3 h. After completion, the crude product was passed through a SiO2 column (60-120 mesh, 15 kg) and eluted with ethyl acetate (40 L). The solvent was evaporated to afford the title compound as a sticky yellow liquid (580 g, 58%). 1H NMR (400 MHz; CDCl3): └ 6.13 (d, J=4.4 Hz, 1H), 4.35-4.42 (m, 3H), 4.01-4.07 (m, 2H), 1.54 (s, 3H), 1.44 (s, 3H), 1.36 (s, 3H), 1.31 (s, 3H).

### Step-2

### (3aR,6aR)-5-((S)-2,2-dimethyl-1,3-dioxolan-4-yl)-2,2-dimethyl-3a,6a-dihydro furo[2,3-d][1,3]dioxol-6-yl acetate

To a stirred solution of (3aR,5R,6aS)-5-((S)-2,2-dimethyl-1,3-dioxolan-4-yl)-2,2-dimethyldihydrofuro [2,3-d][1,3]dioxol-6(5H)-one (580 g, in ACN and pyridine was added Ac20 and the reaction mixture was heated to 80° C. for 16 h. After consumption of the starting material (monitored by TLC), the reaction mixture was concentrated in vacuum and codistilled with toluene (3×250 mL) to afford the title compound as a dark brown sticky liquid (595 g, crude, 88%).

1H-NMR (400 MHz; CDCl3): └ 6.03-6.02 d, 1H), 5.39-5.38 (d, J=5.4 Hz, 1H), 4.7 (t, 1H), 4.0-4.10 (m, 2H), 2.23 (s, 3H), 1.54 (s, 3H), 1.46 (s, 3H), 1.44 (s, 3H), 1.37 (s, 3H).

### Step-3

### (3aR,5S,6R,6aR)-5-((R)-2,2-dimethyl-1,3-dioxolan-4-yl)-2,2-dimethyl tetrahydrofuro [2,3-d][1,3]dioxol-6-yl acetate

To a stirred solution of (3aR,6aR)-5-((S)-2,2-dimethyl-1,3-dioxolan-4-yl)-2,2-dimethyl-3a,6a-dihydro furo[2,3-d][1,3]dioxol-6-yl acetate (595 g) in EtOAc (8 volume) was added 10% Pd/C (200 g, 50% wet) and the reaction mixture was stirred at 40° C. for 12 h under H2 atm (80 psi). After completion, the reaction mixture was filtered through celite, washed with EtOAc (5×300 mL) and concentrated in vacuum to afford the title compound as a sticky yellow liquid (544 g, 91%). 1H NMR (400 MHz; CDCl3): └ 5.80 (d, J=4.0 Hz, 1H), 5.04 (t, J=12.3 Hz, 1H), 4.78-4.81 (m, 1H), 4.58-4.64 (m, 1H), 4.01-4.13 (m, 2H), 3.5 (t, J=15.7 Hz, 1H), 2.16 (s, 3H), 1.57 (s, 3H), 1.43 (s, 3H), 1.37 (s, 3H), 1.34 (s, 3H).

### Step-4

### (3aR,5R,6R,6aR)-5-((R)-2,2-dimethyl-1,3-dioxolan-4-yl)-2,2-dimethyltetrahydrofuro[2,3-d][1,3]dioxol-6-ol

To a stirred solution of (3aR,5S,6R,6aR)-5-((R)-2,2-dimethyl-1,3-dioxolan-4-yl)-2,2-dimethyl tetrahydrofuro [2,3-d][1,3]dioxol-6-yl acetate (544.0 g) in MeOH:H2O (1900 mL:1900 mL) was added Et3N (3.0 eq) and the reaction mixture was stirred at rt for 3.5 h. After completion, the reaction mixture was concentrated in vacuum and codistilled with toluene (3×500 mL) to afford the title compound as a black solid (510 g, crude). The crude was used for next step without purification. 1H NMR (400 MHz; CDCl3): └ 5.78 (d, J=4.0 Hz, 1H), 4.66 (t, J=10.2 Hz, 1H), 4.44-4.50 (m, 1H), 4.2 (m, 1H), 3.9 (m, 1H), 3.03-3.09 (m, 1H), 3.70 (t, J=4.5 Hz, 1H), 1.44 (s, 3H), 1.42 (s, 3H), 1.37 (s, 3H).

### Steps-5 & 6

### (3aR,5R,6S,6aR)-6-azido-5-((R)-2,2-dimethyl-1,3-dioxolan-4-yl)-2,2-dimethyl tetrahydrofuro[2,3-d][1,3]dioxole

To a stirred solution of (3aR,5R,6R,6aR)-5-((R)-2,2-dimethyl-1,3-dioxolan-4-yl)-2,2-dimethyltetrahydrofuro[2,3-d][1,3]dioxol-6-ol (510 g) in DCM:pyridine (3.0 eq) was slowly added triflic anhydride in DCM at −20° C. and the reaction mixture was stirred at same temperature for 30 min. after completion, the reaction mixture was quenched with ice cold 1N HCl (pH ˜6) and the aqueous layer was extracted with DCM (2×1000 mL), dried (Na2SO4) and concentrated. This crude residue was dissolved in DMF and NaN3 (5.0 eq) was added portionwise at 0° C. and stirred at the same temperature for 3 h. After completion, the reaction mixture was poured into ice water (500 mL) and extracted with ethyl acetate (2×1000 mL). The combined organic layer was again washed with ice cold water (3×500 mL), dried (Na2SO4) and concentrated. The residue was purified by flash column chromatography [normal phase, silica gel (100-200 mesh), gradient 0 to 5% EtOAc in hexane] to afford the title compound as pale yellow gum (150 g, 27%). 1H NMR (400 MHz, CDCl3): └ 5.80 (d, J=3.8 Hz, 1H), 4.60-4.63 (m, 1H) 4.35-4.39 (m, 1H), 4.10 (t, J=3.6 Hz, 1H), 3.94 (d, J=2.8 Hz, 1H), 3.89-3.93 (m, 2H), 1.58 (s, 3H), 1.55 (s, 3H), 1.45 (s, 3H), 1.36 (s, 3H).

### Step-7

### Synthesis of (3R,4S,5R,6R)-4-azido-6-(hydroxymethyl)tetrahydro-2H-pyran-2,3,5-triol

To a solution of (3aR,5R,6S,6aR)-6-azido-5-((R)-2,2-dimethyl-1,3-dioxolan-4-yl)-2,2-dimethyl tetrahydrofuro [2,3-d][1,3]dioxole (150 g) in DCM (500 mL) 90% TFA in water was slowly added at −20° C. and stirred at same temperature for 15 min. After completion the reaction mixture was concentrated in vacuum and codistilled with toluene (3×500 mL) to afford (3R,4S,5R,6R)-4-azido-6-(hydroxymethyl)tetrahydro-2H-pyran-2,3,5-triol (100 g, 94%) as a yellow solid.

1H-NMR (400 MHz; CDCl3): └ 5.23 (d, J=3.44 Hz, 1H), 4.59 (d, J=7.64 Hz, 1H), 4.64 (t, J=15.7 Hz, 2H), 3.87-3.93 (m, 1H), 3.58 (t, J=17.7 Hz, 1H), 3.48-3.56 (m, 1H).

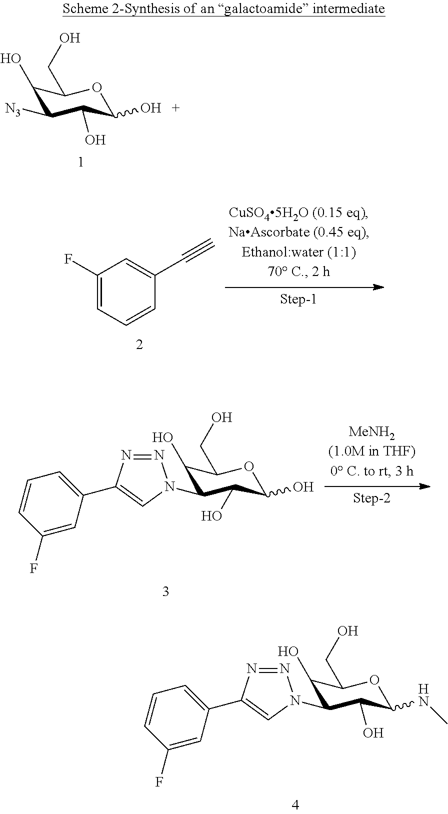

### Step-1

### (3R,4S,5R,6R)-4-(4-(3-fluorophenyl)-1H-1,2,3-triazol-1-yl)-6-(hydroxymethyl) tetrahydro-2H-pyran-2,3,5-triol (3)

CuSO4.5H2O (638 mg, 1.64 mmol) and sodium ascorbate (870 mg, 4.39 mmol) were added to a solution of (3R,4S,5R,6R)-4-azido-6-(hydroxymethyl)tetrahydro-2H-pyran-2,3,5-triol (2.0 g, 9.75 mmol) and 1-ethynyl-3-fluorobenzene (2.46 g, 19.51 mmol) in EtOH-H2O (1:1, 20 mL) at room temperature and the reaction mixture was heated to 70° C. for 5 h. After completion, the reaction mixture was cooled to room temperature. The volatiles were evaporated and the aqueous part was extracted with EtOAc (3×30 mL). The organic layer was dried (Na2SO4) and concentrated and the residue was triturated with Et2O to afford the title compound as a yellow solid (2.8 g, 90%). ESIMS m/z 326 [M+H]+; 1H NMR (400 MHz, DMSO-d6, anomeric mixture, α:β=1:1): d 3.37-3.46 (m, 3H), 3.49-3.57 (m, 3H), 3.66 (t, J=6.1 Hz, 1H), 3.86-3.89 (m, 1H), 3.92-3.96 (m, 2H), 4.03 (t, J=6.2 Hz, 1H), 4.25-4.32 (m, 1H), 4.53-4.61 (m, 2H), 4.66 (t, J=5.5 Hz, 1H), 4.71 (dd, J=11.0 & 3.1 Hz, 1H), 4.84-4.89 (m, 2H), 5.11-5.17 (m, 3H), 5.23 (d, J=5.7 Hz, 1H), 6.71 (d, J=4.5 Hz, 1H), 6.93 (d, J=6.1 Hz, 1H), 7.14 (t, J=8.5 Hz, 2H), 7.46-7.51 (m, 2H), 7.69 (d, J=10.2 Hz, 2H), 7.73 (d, J=7.8 Hz, 2H), 8.57 (s, 1H), 8.61 (s, 1H).

### Step-2

### (2R,3R,4S,5R)-4-(4-(3-fluorophenyl)-1H-1,2,3-triazol-1-yl)-2-(hydroxymethyl)-6-(methylamino)tetrahydro-2H-pyran-3,5-diol (4)

Methyl amine (1.0 M in THF, 10.0 mL) was added a solution of (3R,4S,5R,6R)-4-(4-(3-fluorophenyl)-1H-1,2,3-triazol-1-yl)-6-(hydroxymethyl)tetrahydro-2H-pyran-2,3,5-triol (950 mg, 2.91 mmol) in THF (4 mL), at 0° C. The resulting reaction mixture was stirred at rt for 3 h. After completion the volatiles were evaporate under reduced pressure to the title compound as a greenish solid (900 mg, crude). ESIMS m/z 347.12 [M+H]+; 1H NMR (400 MHz, CDCl3): └ 2.04 (s, 3H), 2.06 (s, 3H), 2.18 (s, 3H), 2.45 (s, 3H), 2.76-2.80 (m, 1H), 4.03-4.17 (m, 3H), 5.44-5.53 (m, 3H), 7.27 (d, J=8.1 Hz, 2H), 7.75 (d, J=8.1 Hz, 2H).

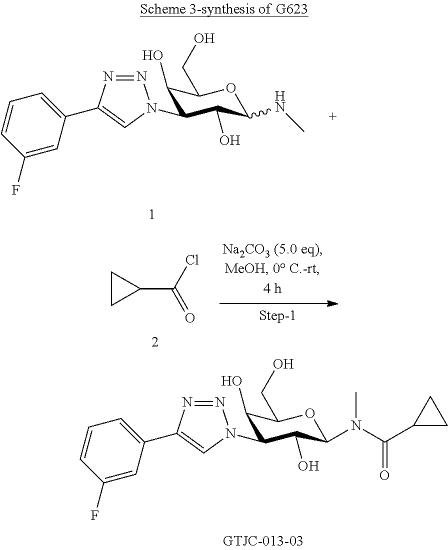

### Step-1

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylcyclopropanecarboxamide (GTJC-013-03)

Na2CO3 (235 mg, 2.212 mmol) and cyclopropanecarbonyl chloride 2 (94 mg, 0.885 mmol) were added to a solution of (2R,3R,4S,5R)-4-(4-(3-fluorophenyl)-1H-1,2,3-triazol-1-yl)-2-(hydroxymethyl)-6-(methylamino)tetrahydro-2H-pyran-3,5-diol 1 (150 mg, 0.442 mmol) in methanol (3 mL) at 0° C. The reaction mixture was stirred at room temperature. After completion the reaction mixture was quenched with water (5 mL) and extracted with EtOAc (3×25 mL). The combined organic layers were washed with brine, dried (Na2SO4), filtered and concentrated under reduced pressure at 45° C. The residue was purified by flash column chromatography eluting with 4% Methanol in DCM to afford the title compound as a white solid (35 mg, 19%). HRMS: (ESI) [M+H]+ calc. for C19H23FN4O5 406.17, found: 407.36 [M+H]+; LCMS: m/z 407 [M+H]+ (ES+) at 89.73% at 3.92 min and 7.08% at 4.14 min.

1H NMR (400 MHz, DMSO-d6, anomeric mixture, α:β=1:9): └ 8.72 (s, 1H), 7.69-7.76 (m, 2H), 7.43-7.52 (m, 1H), 7.13-7.17 (m, 1H), 5.54-5.57 (m, 1H), 5.34 (d, 0.9H, J1-2=6.4 Hz, α-H-1), 5.33 (d, 0.1H, J1-2=2.7 Hz, β-H-1), 4.96-5.00 (m, 1H), 4.48-4.82 (m, 2H), 3.74-3.92 (m, 2H), 3.48-3.53 (m, 2H), 3.13 (s, 3H), 2.08 (m, 1H), 0.75-0.85 (m, 4H),

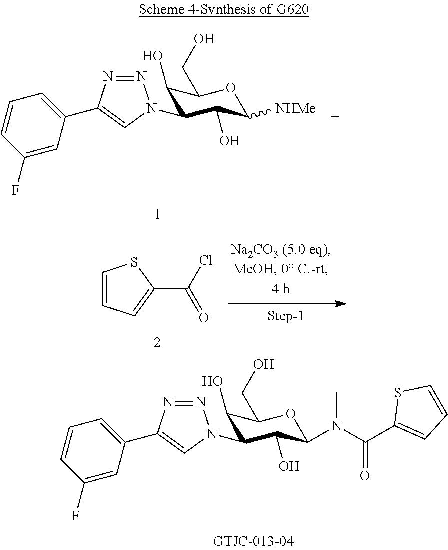

### Step-1

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylthiophene-2-carboxamide (GTJC-013-04)

Na2CO3 (47.04 mg, 0.4437 mmol) and thiophene-2-carbonyl chloride 2 (43.19 mg, 0.2958 mmol) were added to a solution of (2R,3R,4S,5R)-4-(4-(3-fluorophenyl)-1H-1,2,3-triazol-1-yl)-2-(hydroxymethyl)-6-(methylamino)tetrahydro-2H-pyran-3,5-diol 1 (50 mg, 0.1479 mmol) in methanol (3 mL) at 0° C. The reaction mixture was stirred at room temperature. After completion the reaction mixture was quenched with water (5 mL) and extracted with EtOAc (3×25 mL). The combined organic layers were washed with brine, dried (Na2SO4), filtered and concentrated under reduced pressure at 45° C. The residue was purified by flash column chromatography by using 2% Methanol in DCM to afford the title compound as a white solid (15 mg, 23%). HRMS (ESI) [M+H]+ calc. for C20H21FN4O5S: 448.12, found: 449.35 [M+H]+: LCMS: m/z 449 [M+H]+;

1H NMR (400 MHz, DMSO-d6, single β isomer): └ 8.66 (s, 1H), 7.83 (d, 1H), 7.75-7.82 (m, 2H), 7.63 (d, 1H), 7.47-7.52 (m, 1H), 7.13-7.17 (m, 2H), 5.60 (s, 1H), 5.35 (d, J1-2=6.5 Hz, α-H-1), 5.19 (s, 1H), 4.91-4.94 (m, 1H), 4.86 (m, 1H), 4.52-4.54 (m, 1H), 3.90 (m, 1H), 3.79 (m, 1H), 3.52-3.56 (m, 2H), 3.06 (s, 3H).

### Synthesis of G617

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methyl-2-naphthamide (GTJC-013-08)

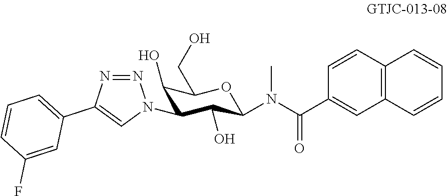

Synthesized following the standard procedure used for GTJC-013-03 or GTJC-013-04

Appearance: white solid; synthesized: 85 mg; yield 23%

LCMS: m/z 493 [M+H]+; 1H NMR (400 MHz, DMSO-d6): └ 3.13 (s, 3H), 3.52-3.60 (m, 2H), 3.66-3.72 (m, 1H), 3.79-3.83 (m, 1H), 4.50-4.56 (m, 1H), 4.72-4.77 (m, 1H), 4.85 (d, J=8.8 Hz, 1H), 5.01 (t, J=5.3 Hz, 1H), 5.33 (d, J=6.8 Hz, 1H), 5.55 (d, J=6.4 Hz, 1H), 7.13-7.18 (m, 1H), 7.46-7.52 (m, 1H), 7.58-7.74 (m, 5H), 7.99 (d, J=8.0 Hz, 2H), 8.05 (d, J=7.6 Hz, 1H), 8.25 (s, 1H), 8.71 (s, 1H).

### Synthesis of G627

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methyl-3-(trifluoromethyl)benzamide (GTJC-013-09)

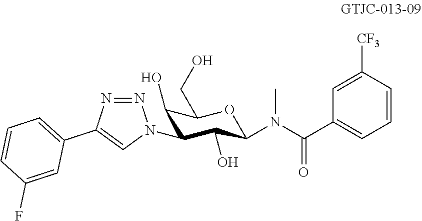

Synthesized following the standard procedure used for GTJC-013-03 or GTJC-013-04

Appearance: white solid; synthesized: 40 mg; yield 13%

HRMS (ESI) [M+H]+ calc. for C23H22F4N4O5 510.15, found: 511.37 [M+H]+

1H NMR (400 MHz, DMSO-d6) (anomeric mixture α:β=1:8): └ 8.73 (s, 1H), 7.87-7.93 (m, 3H), 7.67-7.74 (m, 3H), 7.47-7.52 (m, 1H), 7.13-7.18 (m, 1H), 5.60 (d, J1-2=6.68 Hz, α-H-1), 5.34 (d, 1H), 5.01 (d, J1-2=4.7 Hz, β-H-1), 4.43-4.83 (m, 4H), 3.53-3.58 (m, 4H), 3.08 (s, 3H).

### Synthesis of G628

### 3,4-difluoro-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylbenzamide (GTJC-013-10)

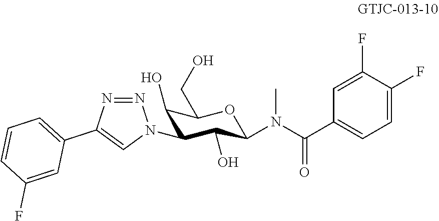

Synthesized following the standard procedure used for GTJC-013-03 or GTJC-013-04

Appearance: white solid; synthesized: 25 mg; yield 9%

α, β isomers are separated by Prep HPLC.

LCMS (β isomer): m/z 479 [M+H]+ (ES+), at 4.65 min (98.44%)

LCMS (α isomer): m/z 479 [M+H]+ (ES+), at 4.79 min (97.98%)

1H-NMR (400 MHz, DMSO-d6, single β isomer) └ 8.73 (s, 1H), 7.72-7.74 (m, 1H), 7.62-7.67 (m, 1H), 7.57-7.60 (m, 1H), 7.50-7.54 (m, 2H), 7.42-7.47 (m, 1H), 7.12-7.17 (m, 1H), 5.58-5.62 (m, 1H), 5.34 (d, 1H, J=6.68 Hz), 4.82-4.99 (m, 2H), 4.78 (d, 1H, J1-2=11.9 Hz, α-H-1), 4.45-4.52 (m, 1H), 3.82-3.96 (m, 1H), 3.49-3.60 (m, 3H), 3.05 (s, 3H).

1H-NMR (400 MHz; DMSO-d6, single α isomer) └ 9.00 (s, 1H), 7.72-7.74 (m, 1H), 7.66-7.68 (m, 1H), 7.49-7.62 (m, 3H), 7.42 (m, 1H), 7.16-7.21 (m, 1H), 6.21 (bs, 1H), 5.17-5.29 (m, 3H), 4.78-4.83 (m, 1H), 4.62-4.64 (m, 1H), 4.45 (d, 1H, J1-2=8.12 Hz, β-H-1), 3.38-3.41 (m, 2H), 3.28-3.33 (m, 2H), 3.05 (s, 3H).

### Synthesis of G622

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methyl-1H-indole-2-carboxamide (GTJC-013-11)

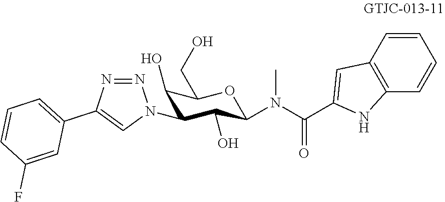

Synthesized following the standard procedure used for GTJC-013-03 or GTJC-013-04

Appearance: white solid: synthesized: 12 mg; yield 8%

HRMS (ESI) [M+H]+ calc. for C24H24FN5O5 481.18, found: 482.38 [M+H]+; LCMS: m/z 482 (M+H)+ (ES+) 93.64% at 4.78 min; 1H NMR (400 MHz, DMSO-d6, single β isomer): └ 11.58 (s, 1H), 8.77 (s, 1H), 7.71-7.73 (m, 1H), 7.62-7.68 (m, 2H), 7.49-7.52 (m, 1H), 7.44-7.47 (m, 1H), 7.19-7.22 (m, 1H), 7.13-7.17 (m, 1H), 7.06-7.08 (m, 1H), 7.03 (s, 1H), 5.60 (s, 2H), 5.36 (d, 1H, J1-2=6.52 Hz, α-H-1), 4.94 (m, 2H), 4.55 (m, 1H), 3.84-3.91 (m, 2H), 3.55-3.64 (m, 2H), 3.10 (s, 3H).

### Synthesis of G641

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methyl-2-phenylacetamide (GTJC-013-27)

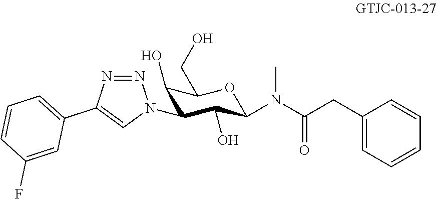

Synthesized following the standard procedure used for GTJC-013-03 or GTJC-013-04

Appearance: white solid; synthesized: 30 mg; yield 23%

ESIMS: m/z 347.12 [M+1]+; 1H NMR (400 MHz, DMSO-d6): └ 3.49-3.61 (m, 4H), 3.72 (t, J=6.2 Hz, 2H), 3.99 (dd, 6.6 & 2.9 Hz, 2H), 4.36-4.43 (m, 2H), 4.70 (t, J=5.5 Hz, 1H), 4.82 (dd, 10.5 & 2.8 Hz, 2H), 5.19 (d, J=9.7 Hz, 2H), 5.31 (d, J=7.2 Hz, 2H), 5.40 (d, J=6.6 Hz, 2H), 7.12-7.17 (m, 2H), 7.46-7.51 (m, 2H), 7.66 (dd, J=10.2 & 2.3 Hz, 2H), 7.72 (d, J=7.8 Hz, 2H), 8.67 (s, 2H).

### Synthesis of G649

### 2-(3,4-difluorophenyl)-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylacetamide (GTJC-013-37)

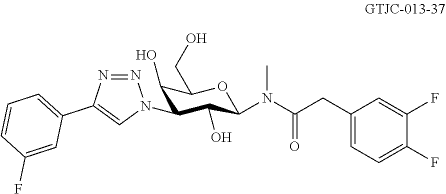

Synthesized following the standard procedure used for GTJC-013-03 or GTJC-013-04

Appearance: white solid; synthesized: 25 mg; yield 17%

HRMS (ESI) [M+H]+ calc. for C23H23F3N4O5 492.16, found: 493.5 [M+H]+

LCMS: m/z 493.5 [M+H]+ (ES+) 88.40% at 4.75 min and 9.81% at 4.88 min,

1H-NMR (400 MHz; DMSO-d6, anomeric mixture, α:β=1:9): └ 8.74 (s, 1H), 7.69-7.76 (m, 2H), 7.49-7.53 (m, 1H), 7.31-7.39 (m, 2H), 7.28-7.29 (m, 2H), 5.59 (d, 1H, J1-2=9.48 Hz, α-H-1), 4.70-5.36 (m, 5H), 3.76-3.94 (m, 4H), 3.45-3.51 (m, 2H), 3.03 (s, 3H).

### Synthesis of G651

### 2-(3,4-difluorophenoxy)-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylacetamide (GTJC-013-38)

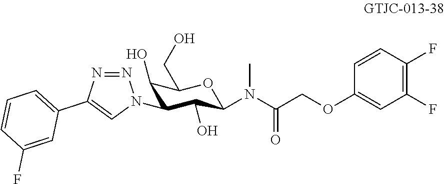

Synthesized following the standard procedure used for GTJC-013-03 or GTJC-013-04

Appearance: white solid; synthesized: 15 mg; yield 11%

HRMS (ESI) [M+H]+ calc. for C23H23F3N4O6 508.16, found: 509.52 [M+H]+

LCMS: m/z 509.5 [M+H]+ (ES+) 91.95% at 4.86 min & 6.98% at 4.96 min,

1H NMR (400 MHz; DMSO-d6, anomeric mixture α:β=1:13): └ 8.77 (s, 2H), 7.74-7.76 (m, 2H), 7.69-7.72 (m, 2H), 7.47-7.53 (m, 2H), 7.30-7.37 (m, 2H), 7.13-7.18 (m, 2H), 7.02-7.08 (m, 2H), 6.76-6.80 (m, 2H), 5.73 (d, 1H, J1-2=6.8 Hz, α-H-1), 5.37-5.41 (m, 2H), 5.29-5.31 (m, 1H), 5.02-5.04 (m, 1H), 4.91-4.98 (m, 5H), 4.76-4.79 (m, 2H), 4.70-4.72 (m, 1H), 4.37-4.44 (m, 2H), 3.91-3.96 (m, 3H), 3.79-3.82 (m, 1H), 3.50-3.56 (m, 2H), 3.00 (s, 2H), 2.88 (s, 3H).

### Synthesis of G652

### 3-(3,4-difluorophenyl)-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylpropanamide (GTJC-013-41)

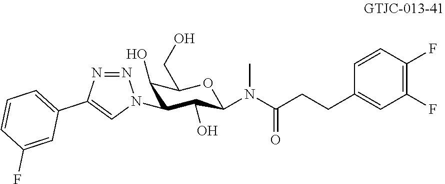

Synthesized following the standard procedure used for GTJC-013-03 or GTJC-013-04

Appearance: white solid; synthesized: 11 mg; yield 9%

HRMS (ESI) [M+H]+ calc. for C24H25F3N4O5 506.18, found: 507.52 [M+H]+; LCMS: m/z 507.5 [M+H]+ (ES+) 75.38% at 5.04 min, 7.07% at 5.15 min, 7.07%, 15.93% at 5.28 min.

1H NMR (400 MHz; DMSO-d6, mixture of 3 isomers): └ 8.71 (s, 1H), 7.51-7.77 (m, 2H), 7.37-7.49 (m, 1H), 7.29-7.35 (m, 2H), 7.14-7.26 (m, 2H), 5.48 (d, 1H, J1-2=6.92 Hz, α-H-1), 4.68-5.32 (m, 4H), 4.40-4.50 (m, 1H), 3.51-3.99 (m, 4H), 2.54-2.89 (m, 7H).

### Synthesis of G658

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-3,4-dimethoxy-N-methylbenzamide (GTJC-013-46-1)

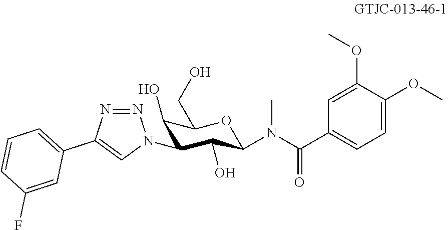

Synthesized following the standard procedure used for GTJC-013-03 or GTJC-013-04

Appearance: white solid; synthesized: 110 mg; yield 37%

HRMS (ESI) [M+H]+ calc. for C24H27FN4O7 502.19, found: 503.52 [M+H]+; LCMS: m/z 503.5 (M+H)+ (ES+) 95.21% at 4.25 min; 1H NMR (400 MHz; DMSO-d6, single β isomer): 8.71 (s, 1H), 7.68-7.75 (m, 2H), 7.46-7.52 (m, 1H), 7.12-7.18 (m, 3H), 7.01 (d, J=8.28 Hz, 1H), 5.55 (d, 1H, J1-2=6.36 Hz, α-H-1), 5.32 (d, J=6.64 Hz, 1H), 4.90-4.92 (m, 1H), 4.83 (m, 2H), 4.45-4.52 (m, 1H), 3.79-3.85 (m, 7H), 3.61-3.67 (m, 1H), 3.32-3.59 (m, 2H), 3.02 (s, 3H).

### Synthesis of G655

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-3,4-dihydroxy-N-methylbenzamide (GTJC-013-46)

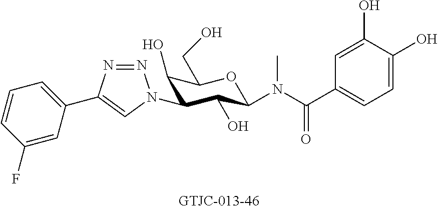

Synthesized following the standard procedure used for GTJC-013-23

Appearance: white solid; synthesized: 18 mg; yield 24%

HRMS (ESI) [M+H]+ calc. for C22H23FN4O7 474.16, found: 475.50 [M+H]+

LCMS: m/z 475.5 [M+H]+ (ES+) 98.93% at 3.86 min.

1H NMR (400 MHz; DMSO-d6, single β isomer): └ 9.11 (bs, 2H), 8.71 (s, 1H), 7.74 (d, 1H, J=7.68 Hz,), 7.69 (d, J=10.32 Hz, 1H), 7.46-7.52 (m, 1H), 7.12-7.17 (m, 1H), 6.94 (s, 2H), 6.73-6.75 (m, 1H), 5.51 (d, 1H, J1-2=6.01 Hz, α-H-1), 5.29 (bs, 1H), 4.45-4.85 (m, 4H), 3.86 (bs, 1H), 3.57 (m, 3H), 2.98 (s, 3H).

### Synthesis of G642

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methyl-3-(trifluoromethoxy)benzamide (GTJC-013-45)

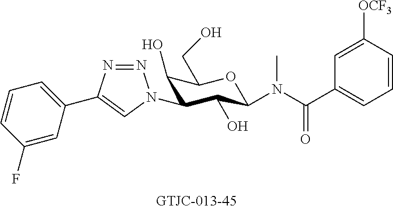

Synthesized following the standard procedure used for GTJC-013-03 or GTJC-013-04

Appearance: white solid; synthesized: 50 mg; yield 33%

HRMS (ESI) [M+H]+ calc. for C23H22F4N4O6 526.15, found: 527.47 [M+H]+

1H NMR (400 MHz, DMSO-d6, anometic mixture): d 8.82 (s, 1H), 7.83-7.93 (m, 3H), 7.69-7.77 (m, 3H), 7.49-7.52 (m, 1H), 7.13-7.18 (m, 1H), 5.60 (d, 1H, J1-2=6.68 Hz, α-H-1), 5.34 (d, J=6.6 Hz, 1H), 5.14 (d, 1H, J1-2=4.0 Hz, β-H-1), 4.47-4.61 (m, 4H), 3.39-3.60 (m, 4H), 3.08 (s, 3H).

### Synthesis of G650

### Synthesis of 2,3,4,5,6-pentafluoro-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylbenzamide (GTJC-013-47)

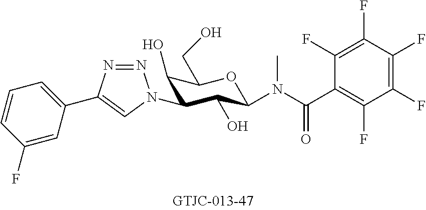

Synthesized following the standard procedure used for GTJC-013-03 or GTJC-013-04

Appearance: white solid; synthesized: 30 mg; yield 19%

HRMS (ESI) [M+H]+ calc. for: C22H18F6N4O5 532.12, found: 533.48 [M+H]+; LCMS: m/z 533.4 [M+H]+ (ES+) 82.08% at 5.04 min & 14.98% at 5.15 min.

1H NMR (400 MHz, DMSO-d6, anomeric mixture, α:β=1:6): └ 8.71 (s, 1H), 7.55-7.74 (m, 2H), 7.47-7.52 (m, 1H), 7.13-7.21 (m, 1H), 5.37 (d, 1H, J1-2=6.6 Hz, α-H-1), 4.37-5.62 (m, 5H), 3.32-3.69 (m, 4H), 3.07 (s, 3H).

### Synthesis of G629

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-1-methoxy-N-methyl-2-naphthamide (GTJC-013-22)

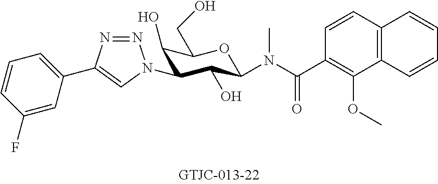

Synthesized following the standard procedure used for GTJC-013-03 or GTJC-013-04

Appearance: white solid; synthesized: 110 mg; yield 35%

ESIMS: m/z 347.12 [M+H]+; 1H NMR (400 MHz, DMSO-d6): └ 3.49-3.61 (m, 4H), 3.72 (t, J=6.2 Hz, 2H), 3.99 (dd, 6.6 & 2.9 Hz, 2H), 4.36-4.43 (m, 2H), 4.70 (t, J=5.5 Hz, 1H), 4.82 (dd, 10.5, 2.8 Hz, 2H), 5.19 (d, J=9.7 Hz, 2H), 5.31 (d, J=7.2 Hz, 2H), 5.40 (d, J=6.6 Hz, 2H), 7.12-7.17 (m, 2H), 7.46-7.51 (m, 2H), 7.66 (dd, J=10.2 & 2.3 Hz, 2H), 7.72 (d, J=7.8 Hz, 2H), 8.67 (s, 2H).

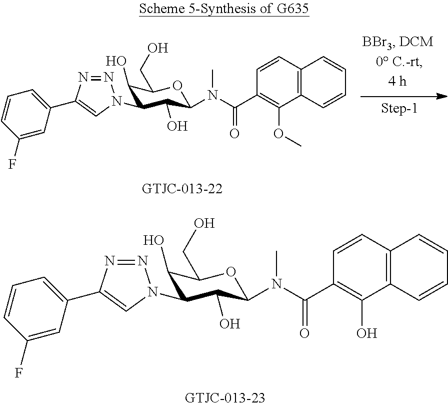

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-1-hydroxy-N-methyl-2-naphthamide (GTJC-013-23)

To a solution of N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-1-methoxy-N-methyl-2-naphthamide (80 mg, 0.1532 mmol) in DCM (4 mL) added BBr3 (115.4 mg, 0.4597 mmol) at 0° C. The resulting mixture was stirred at room temperature for 3 h. After completion, the reaction mixture was quenched with saturated NaHCO₃ solution (6 mL) to adjust pH-8 and the aqueous layer was extracted with DCM (3×10 mL). The combined organic layer was dried (Na2SO4) and concentrated in vacuo. The residue was purified by Flash chromatography eluting with 3% Methanol in DCM to afford the title compound as white solid (13 mg, 17%). ESIMS: m/z 347.12 [M+1]+; 1H NMR (400 MHz, DMSO-d6): └ 3.49-3.61 (m, 4H), 3.72 (t, J=6.2 Hz, 2H), 3.99 (dd, 6.6, 2.9 Hz, 2H), 4.36-4.43 (m, 2H), 4.70 (t, J=5.5 Hz, 1H), 4.82 (dd, 10.5 & 2.8 Hz, 2H), 5.19 (d, J=9.7 Hz, 2H), 5.31 (d, J=7.2 Hz, 2H), 5.40 (d, J=6.6 Hz, 2H), 7.12-7.17 (m, 2H), 7.46-7.51 (m, 2H), 7.66 (dd, J=10.2 & 2.3 Hz, 2H), 7.72 (d, J=7.8 Hz, 2H), 8.67 (s, 2H).

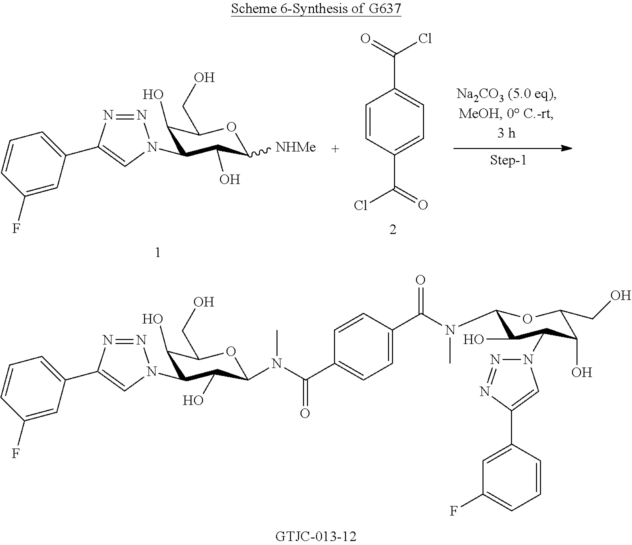

### N1,N4-bis((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N1,N4-dimethylterephthalamide (GTJC-013-12)

To a solution of (2R,3R,4S,5R)-4-(4-(3-fluorophenyl)-1H-1,2,3-triazol-1-yl)-2-(hydroxymethyl)-6-(methylamino)tetrahydro-2H-pyran-3,5-diol (140 mg, 0.4142 mmol) in methanol (3 mL) was added Na2CO3 (220 mg, 2.0710 mmol) and terephthaloyl dichloride (172 mg, 0.8284 mmol) at 0° C. The reaction mixture was stirred at room temperature. After completion the reaction mixture was quenched with water (5 mL) and extracted with EtOAc (3×25 mL). The combined organic layers were washed with brine and dried (Na2SO4), filtered and concentrated under reduced pressure at 45° C. The residue was purified by Prep HPLC to afford the title compound (3 mg) as white solid. HRMS (ESI) [M+H]+ calc. for C38H40F2N8O10 806.28, found: 807.71 [M+H]+; LCMS: m/z 807.7 (M+H)+ (ES+) 98.10% at 4.49 min. 1H NMR (400 MHz; DMSO-d6, 3 isomer): └ 8.73 (s, 2H), 7.65-7.74 (m, 8H), 7.47-7.52 (m, 2H), 7.13-7.17 (m, 2H), 5.64 (d, 2H, J1-2=6.48 Hz, α-H-1), 5.34-5.36 (m, 2H), 4.76-4.81 (m, 4H), 4.49-4.53 (m, 2H), 3.61-3.73 (m, 2H), 3.54-3.58 (m, 6H), 3.08 (s, 6H).

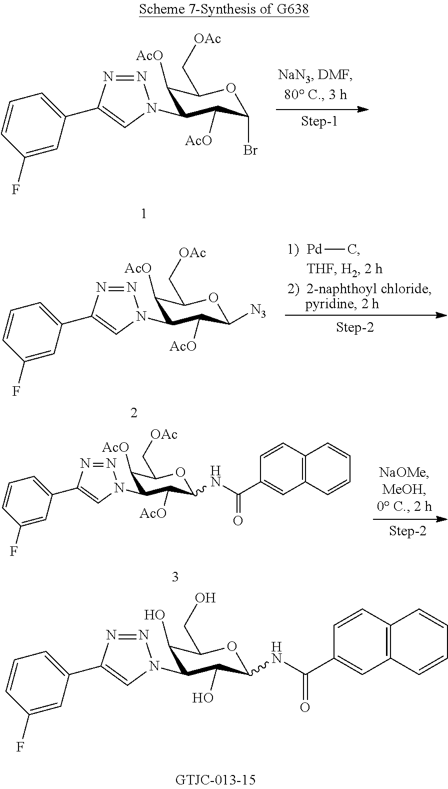

### Step-1

### (2R,3R,4S,5R,6R)-2-(acetoxymethyl)-6-azido-4-(4-(3-fluorophenyl)-1H-1,2,3-triazol-1-yl)tetrahydro-2H-pyran-3,5-diyl diacetate

To a solution of (2R,3R,4S,5R,6R)-2-(acetoxymethyl)-6-bromo-4-(4-(3-fluorophenyl)-1H-1,2,3-triazol-1-yl)tetrahydro-2H-pyran-3,5-diyl diacetate (1.92 g, 3.74 mmol) in DMF (20 mL), NaN3 (1.21 g, 18.7 mmol) was added at room temperature. The reaction mixture was heated to 80° C. for 3 h. After completion, the reaction mixture was cooled to room temperature and quenched with cold water (20 mL). The aqueous layer was extracted with EtOAc (2×20 mL), dried (Na2SO4) and concentrated in vacuo. The crude residue was purified by flash column chromatography [normal phase, silica gel (100-200 mesh), gradient 0 to 50% EtOAc in hexane] to afford the title compound as a white solid (670 mg, 38%). ESIMS m/z 477 [M+H]+; 1H NMR (400 MHz, CDCl3): δ 1.95 (s, 3H), 2.07 (s, 6H), 4.17-4.24 (i, 3H), 4.81 (d, J=8.5 Hz, 1H), 5.17 (dd. J=11.3 & 3.1 Hz, 1H), 5.59 (d, J=2.9 Hz, 1H), 5.62-5.70 (m, 1H), 7.02-7.06 (m, 1H), 7.35-7.41 (m, 1H), 7.52 (t, J=7.7 Hz, 2H), 7.80 (s, 1H).

### Step-2

### (3R,4S,5R,6R)-2-(2-naphthamido)-6-(acetoxymethyl)-4-(4-(3-fluorophenyl)-1H-1,2,3-triazol-1-yl)tetrahydro-2H-pyran-3,5-diyl diacetate

To a solution of (2R,3R,4S,5R,6R)-2-(acetoxymethyl)-6-azido-4-(4-(3-fluorophenyl)-1H-1,2,3-triazol-1-yl)tetra hydro-2H-pyran-3,5-diyl diacetate (100 mg, 0.21 mmol) in THF (5 mL), Pd—C (20 mg, 10%, dry) was added and the reaction mixture was stirred under H2 (1 atm) at room temperature for 2 h. After completion, pyridine (0.05 mL, 0.63 mmol) was added to the reaction mixture, cooled to 0° C. and 2-naphthoyl chloride (80 mg, 0.42 mmol) was slowly added and stirred at room temperature for 2 h. After completion, the reaction mixture was filtered, washed with EtOAc (3×10 mL). The combined organic layers were washed with water (10 mL), dried (Na2SO4), and concentrated in vacuo to afford the title compound as a white sticky solid (158 mg, crude). ESIMS: m/z 605 [M+H]+.

### Step-3

### N-((3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-2-naphthamide (GTJC-013-15)

To a solution of (3R,4S,5R,6R)-2-(2-naphthamido)-6-(acetoxymethyl)-4-(4-(3-fluorophenyl)-1H-1,2,3-triazol-1-yl)tetrahydro-2H-pyran-3,5-diyl diacetate (4, 158 mg, 0.26 mmol) MeOH (5 mL), NaOMe (0.26 mL, 1M, 0.26 mmol) was added at 0° C. The reaction mixture was stirred at 0° C. for 2 h. After completion, the reaction mixture was acidified with Amberlyst 15 (pH ˜6) and filtered. Washed with MeOH (3×10 mL) and concentrated in vacuo. The residue was purified by flash column chromatography [normal phase, silica gel (100-200 mesh), gradient 0 to 10% MeOH in DCM] to afford the title compound as a white solid (60 mg, 48%). LCMS: m/z 479 (M+H)+; (ES+) 70.96% at 4.80 min and 23.57% at 4.87 min. 1H NMR (400 MHz; DMSO-d6): └ 3.53 (t, J=5.9 Hz, 2H), 3.83 (t, J=6.1 Hz, 1H), 3.97-4.02 (m, 2H), 4.39-4.44 (m, 1H), 4.70-4.74 (m, 1H), 4.93 (dd, J=10.8, 2.8 Hz, 1H), 5.28 (d, J=6.7 Hz, 1H), 5.36 (d, J=6.7 Hz, 1H), 7.16 (td, J=8.8, 2.5 Hz, 1H), 7.46-7.52 (m, 1H), 7.59-7.66 (m, 2H), 7.69-7.76 (m, 2H), 7.96-8.07 (m, 4H), 8.50, 8.60 (each singlet, 1H), 9.27 (d, J=8.9 Hz, 1H).

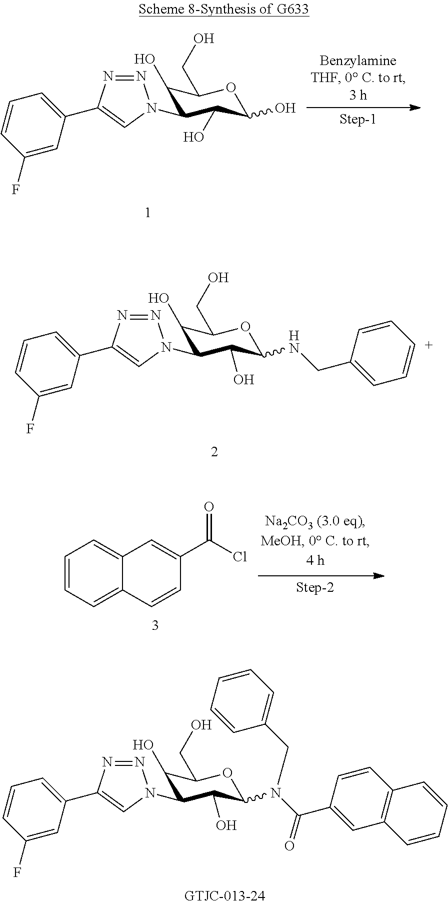

### Step-1

### (3R,4S,5R,6R)-2-(benzylamino)-4-(4-(3-fluorophenyl)-1H-1,2,3-triazol-1-yl)-6-(hydroxymethyl)tetrahydro-2H-pyran-3,5-diol

Benzylamine (87.1 mg, 0.8136 mmol) was added to a solution of (3R,4S,5R,6R)-4-(4-(3-fluorophenyl)-1H-1,2,3-triazol-1-yl)-6-(hydroxymethyl) tetra hydro-2H-pyran-2,3,5-triol (250 mg, 0.7396 mmol) in TH (2 mL) and the mixture was stirred at room temperature for 2 h. After completion, the reaction mixture was concentrated and the residue was triturated by Et2O to afford the title compound as a light yellow solid (150 mg). The crude material was used in next step. ESIMS: m/z 353 [M+H]+.

### Step-2

### N-benzyl-N-((2R,3R,4S,5R,6R-2-(benzylamino)-4-(4-3-fluorophenyl)-1H-1,2,3-triazol-1-yl)-3,5-dihydroxy-6-(hydroxymethyl)tetrahydro-2H-pyran-2-yl)-2-naphthamide

Na2CO3 (115.04 mg, 1.0869 mmol) and 2-naphthoyl chloride (190.99 mg, 0.7246 mmol) were added to a solution of (3R,4S,5R,6R)-2-(benzylamino)-4-(4-(3-fluorophenyl)-1H-1,2,3-triazol-1-yl)-6-(hydroxymethyl)tetrahydro-2H-pyran-3,5-diol (150 mg, 0.3623 mmol) in methanol (3 mL) at 0° C. The reaction mixture was stirred at room temperature. After completion, the reaction mixture was quenched with water (5 mL) and extracted with EtOAc (3×25 mL). The combined organic layers were washed with brine, dried (Na2SO4), filtered and concentrated under reduced pressure at 45° C. The residue was purified by prep HPLC to afford anomeric mixture the title compound as white solid (3 mg). HRMS (ESI) [M+H]+ calc. for C32H29FN4O5 568.21, found: 569.53 [M+H]+; LCMS: m/z 569.5 [M+H]+ (ES+) 96.83% at 5.59 min.

1H NMR (400 MHz; DMSO-d6, single β isomer): └ 8.70 (s, 1H), 8.24 (s, 1H), 8.07-8.08 (m, 1H), 7.98-8.01 (m, 2H), 7.51-7.71 (m, 7H), 7.46-7.51 (m, 1H), 7.31-7.35 (m, 2H), 7.20-7.24 (m, 1H), 7.12-7.17 (m, 1H), 5.66 (bs, 1H), 5.32 (bs, 1H), 5.06 (d, 1H, J1-2=7.72 Hz, α-H-1), 4.86-4.89 (m, 1H), 4.77-4.79 (m, 1H), 4.65-4.69 (m, 1H), 4.54-4.59 (m, 1H), 3.83 (s, 1H), 3.62-3.70 (m, 2H), 3.56-3.58 (m, 1H).

### Synthesis of G639

### N-benzyl-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)benzamide (GTJC-013-20)

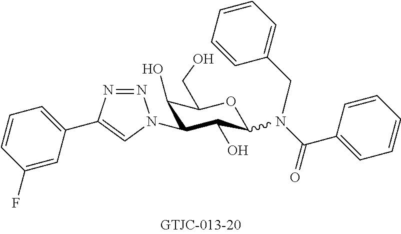

Synthesized following the standard procedure used for GTJC-013-24

Appearance: white solid; synthesized: 1 mg

ESIMS: 949 [M+H]+; LCMS: m/z 697 (M+H)+ (ES+) 96.37% at 4.51 min,

1H NMR (400 MHz, DMSO-d6): └ 3.49-3.61 (m, 4H), 3.72 (t, J=6.2 Hz, 2H), 3.99 (dd, 6.6 & 2.9 Hz, 2H), 4.36-4.43 (m, 2H), 4.70 (t, J=5.5 Hz, 1H), 4.82 (dd, 10.5 & 2.8 Hz, 2H), 5.19 (d, J=9.7 Hz, 2H), 5.31 (d, J=7.2 Hz, 2H), 5.40 (d, J=6.6 Hz, 2H), 7.12-7.17 (m, 2H), 7.46-7.51 (m, 2H), 7.66 (dd, J=10.2 & 2.3 Hz, 2H), 7.72 (d, J=7.8 Hz, 2H), 8.67 (s, 2H).

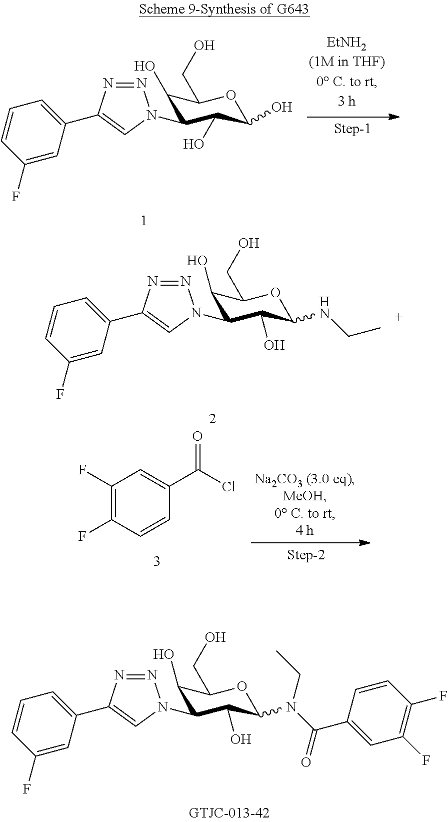

### Step-1

### (3R,4S,5R,6R)-2-(ethylamino)-4-(4-(3-fluorophenyl)-1H-1,2,3-triazol-1-yl)-6-(hydroxymethyl)tetrahydro-2H-pyran-3,5-diol (2)

A solution of (3R,4S,5R,6R)-4-(4-(3-fluorophenyl)-1H-1,2,3-triazol-1-yl)-6-(hydroxymethyl) tetra hydro-2H-pyran-2,3,5-triol (3, 150 mg, 1.53 mmol) in ethylamine (1.0 M in THF, 2 mL) was stirred at room temperature for 2 h. After completion, the reaction mixture was concentrated in vacuo. The resulting crude residue was triturated by Et2O to afford the title compound as a light yellow solid (100 mg, crude). The material was taken for next step without further purification. HRMS (ESI) [M+H]+ calc. for C16H21FN4O4 352.15, found: 353.33 [M+H]+; ESIMS: m/z 353 [M+H]+.

### Step-2

### N-ethyl-3,4-difluoro-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)benzamide (GTJC-013-42)

To a solution of (3R,4S,5R,6R)-2-(ethylamino)-4-(4-(3-fluorophenyl)-1H-1,2,3-triazol-1-yl)-6-(hydroxymethyl)tetrahydro-2H-pyran-3,5-diol (100 mg, 0.2840 mmol) in methanol (3 mL) was added Na2CO3 (90.04 mg, 0.8522 mmol) and 3,4-difluorobenzoyl chloride (99.99 mg, 0.5681 mmol) at 0° C. The reaction mixture was stirred at room temperature. After 3 h, the reaction mixture was quenched with water (5 mL) and extracted with EtOAc (3×25 mL). The combined organic layers were washed with brine and dried (Na2SO4). The solvent was removed under reduced pressure at 45° C. and the residue was purified by flash column chromatography by using 4.5% Methanol in DCM to afford the title compound as white solid (15 mg, 11%).

HRMS (ESI) [M+H]+ calc. for C23H23F3N4O5 492.16, found: 493.47 [M+H]+

LCMS: m/z 493.4 [M+H]+ (ES+), at 5.28 min (93.45%) & 5.39 min (6.26%).

1H NMR (400 MHz; DMSO-d6, anomeric mixture α:β=1:15): └ 8.75 (s, 1H), 7.71-7.76 (m, 1H), 7.65-7.69 (m, 1H), 7.48-7.59 (m, 3H), 7.41-7.47 (m, 1H), 7.12-7.17 (m, 1H), 5.50 (d, 1H, J1-2=6.92 Hz, α-H-1), 5.34 (d, J=6.48 Hz, 1H), 4.62-4.79 (m, 3H), 4.42-4.49 (m, 1H), 3.82 (bs, 1H), 3.66-3.69 (m, 1H), 3.32-3.54 (m, 4H), 1.25 (m, 3H).

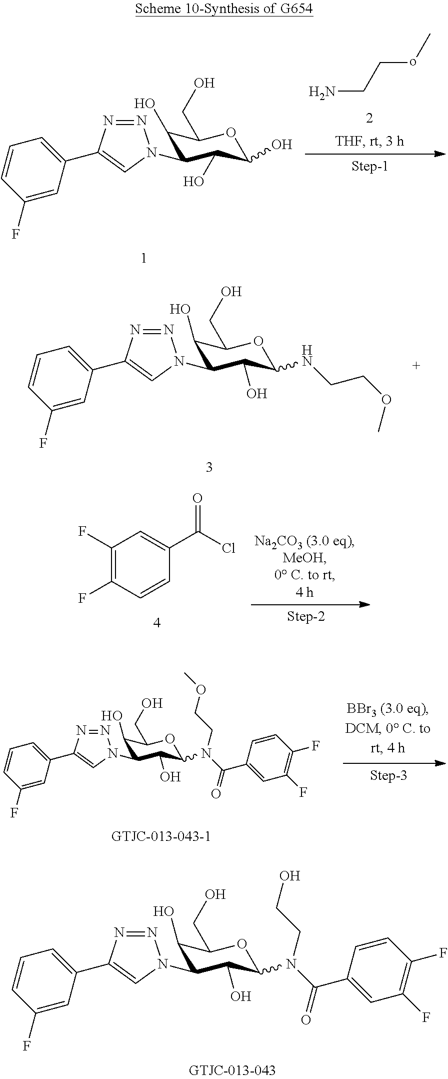

### Step-1

### (2R,3R,4S,5R,6R)-4-(4-(3-fluorophenyl)-1H-1,2,3-triazol-1-yl)-2-(hydroxymethyl)-6-((2-methoxyethyl)amino)tetrahydro-2H-pyran-3,5-diol (3)

2-methoxyethan-1-amine 2 (93.25 mg, 1.226 mmol) was added to a solution of (3R,4S,5R,6R)-4-(4-(3-fluorophenyl)-1H-1,2,3-triazol-1-yl)-6-(hydroxymethyl) tetra hydro-2H-pyran-2,3,5-triol (1, 200 mg, 0.613 mmol) in THF (5 ml) and the mixture was stirred at room temperature for 2 h. After completion, the reaction mixture was concentrated in vacuo and the residue was triturated by Et2O to afford the title compound as a light yellow solid (170 mg, crude. The material was taken for next step. HRMS (ESI) [M+H]+ calc. for C17H23FN4O5 382.17, found: 383.17 [M+H]+; ESIMS: m/z 383 [M+H]+.

### Step-2

### 3,4-difluoro-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-(2-methoxyethyl)benzamide (GTJC-013-43-1)

To a solution of (2R,3R,4S,5R,6R)-4-(4-(3-fluorophenyl)-1H-1,2,3-triazol-1-yl)-2-(hydroxymethyl)-6-((2-methoxyethyl)amino)tetrahydro-2H-pyran-3,5-diol (150 mg, 0.3926 mmol) in methanol (3 mL) was added Na2CO3 (124.8 mg, 1.1780 mmol) and 3,4-difluorobenzoyl chloride (138.02 mg, 0.7853 mmol) at 0° C. The reaction mixture was stirred at room temperature. After 3 h, the reaction mixture was quenched with water (5 mL) extracted with EtOAc (3×25 mL). The combined organic phase was washed with brine and dried (Na2SO4). The solvent was removed under reduced pressure at 45° C. and the residue was purified by flash column chromatography by using 2% Methanol in DCM to afford the title compound as a white solid (110 mg, 54%). HRMS (ESI) [M+H]+ calc. for C24H25F3N4O6 522.17, found: 523.58 [M+H]+

1H NMR (400 MHz, DMSO-d6, with D2O, β isomer): └ 8.76 (s, 1H), 7.71-7.75 (m, 1H), 7.68-7.71 (m, 1H), 7.56-7.68 (m, 3H), 7.47-7.51 (m, 1H), 7.12-7.17 (m, 1H), 5.47 (d, 1H, J1-2=6.44 Hz, α-H-1), 5.36 (d, J=6.56 Hz, 1H), 4.83-4.85 (m, 1H), 4.76-4.78 (m, 2H), 4.43-4.50 (m, 1H), 3.83-3.88 (m, 2H), 3.48-3.56 (m, 6H), 3.29 (s, 3H).

### Step-3

### 3,4-difluoro-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-(2-hydroxyethyl)benzamide (GTJC-013-43)

To a solution of 3,4-difluoro-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-(2-methoxyethyl)benzamide (80 mg, 0.1532 mmol) in DCM (4 mL) BBr3 (115.4 mg, 0.4597 mmol) was added at 0° C. The reaction mixture was stirred at room temperature for 3 h. After completion, the reaction mixture was quenched with saturated NaHCO₃ solution (6 mL) to adjust pH-8 and extracted with DCM (3×25 mL). The combined organic phase was washed with brine and dried (Na2SO4) filtered and concentrated under reduced pressure at 45° C. The residue was purified by prep HPLC to afford the title compound as a white solid (6 mg, 8%).

HRMS (ESI) [M+H]+ calc. for C23H23F3N4O6 508.16, found: 509.5 [M+H]+

LCMS: m/z 509.5 (M+H)+ (ES+) 59.89% at 4.57 min, 29.86% at 4.66 min, 9.66% at 4.76 min.

1H NMR (400 MHz; DMSO-d6, mixture of 3 isomers): └ 8.69 (s, 1H), 7.45-7.77 (m, 6H), 7.13-7.18 (m, 1H), 5.56 (d, 1H, J1-2=6.16 Hz, α-H-1), 5.37 (d, J=6.4 Hz, 1H), 4.41-5.04 (m, 5H), 3.48-4.28 (m, 8H).

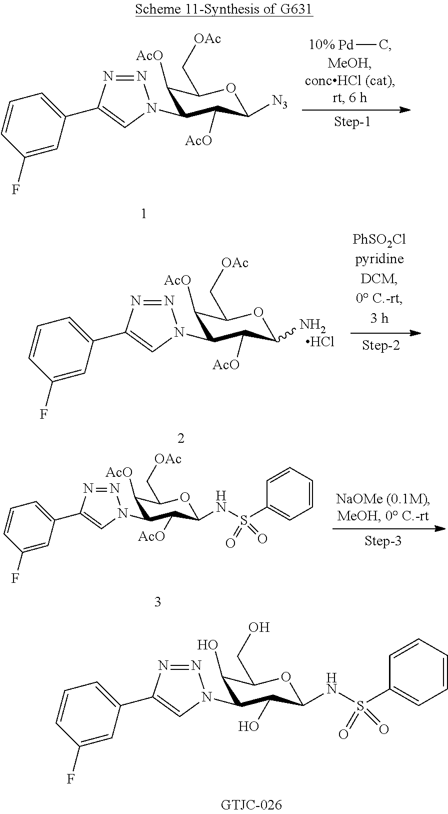

### Step-1

### (2R,3R,4S,5R)-2-(acetoxymethyl)-6-amino-4-(4-(3-fluorophenyl)-1H-1,2,3-triazol-1-yl)tetrahydro-2H-pyran-3,5-diyl diacetate hydrochloride (3)

10% Pd—C (50 mg) and conc. HCl (two drops) were added solution of (2R,3R,4S,5R)-2-(acetoxymethyl)-6-azido-4-(4-(3-fluorophenyl)-1H-1,2,3-triazol-1-yl)tetrahydro-2H-pyran-3,5-diyl diacetate (115 mg, 0.2415 mol) in Methanol (3 mL). The mixture was stirred under hydrogen atmosphere (balloon pressure) at room temperature for 2 h. After completion, the reaction mixture was filtered through celite, washed with methanol (10 mL). The combined filtrate was concentrated in vacuo to afford the title compound as an off white solid (93 mg, 86%). The residue was used in next step without further purification. 1H NMR (400 MHz, CDCl3): └ 2.04 (s, 3H), 2.06 (s, 3H), 2.18 (s, 3H), 2.45 (s, 3H), 2.76-2.80 (m, 1H), 4.03-4.17 (m, 3H), 5.44-5.53 (m, 3H), 7.27 (d, J=8.1 Hz, 2H), 7.75 (d, J=8.1 Hz, 2H).

### Step-2

### (2R,3R,4S,5R,6R)-2-(acetoxymethyl)-4-(4-(3-fluorophenyl)-1H-1,2,3-triazol-1-yl)-6-(phenylsulfonamido)tetrahydro-2H-pyran-3,5-diyl diacetate (4)

To a solution of (2R,3R,4S,5R)-2-(acetoxymethyl)-6-amino-4-(4-(3-fluorophenyl)-1H-1,2,3-triazol-1-yl)tetrahydro-2H-pyran-3,5-diyl diacetate hydrochloride (3, 93 mg 0.1913 mmol) in DCM (5 mL) pyridine (46 mg, 0.5740 mmol) and benzene sulfonyl chloride chloride (50.69 mg, 0.2870 mmol) were added at 0° C. The reaction mixture was stirred at room temperature for 3 h. After completion, the reaction mixture was quenched with water (3 mL) and extracted with DCM (3×15 mL). The combined organic layers were dried (Na2SO4) and concentrated in vacuo to afford the title compound as an off white semi solid (130 mg, crude). HRMS (ESI) [M+H]+ calc. for C26H27FN4O9S 590.15, found: 591.32 [M+H]+; 1H NMR (400 MHz, DMSO-d6): └ 3.49-3.61 (m, 4H), 3.72 (t, J=6.2 Hz, 2H), 3.99 (dd, 6.6 & 2.9 Hz, 2H), 4.36-4.43 (m, 2H), 4.70 (t, J=5.5 Hz, 1H), 4.82 (dd, 10.5 & 2.8 Hz, 2H), 5.19 (d, J=9.7 Hz, 2H), 5.31 (d, J=7.2 Hz, 2H), 5.40 (d, J=6.6 Hz, 2H), 7.12-7.17 (m, 2H), 7.46-7.51 (m, 2H), 7.66 (dd, J=10.2 & 2.3 Hz, 2H), 7.72 (d, J=7.8 Hz, 2H), 8.67 (s, 2H).

### Step-3

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)benzenesulfonamide (GTJC-026)

To a solution of (2R,3R,4S,5R,6R)-2-(acetoxymethyl)-4-(4-(3-fluorophenyl)-1H-1,2,3-triazol-1-yl)-6-(phenylsulfonamido)tetrahydro-2H-pyran-3,5-diyl diacetate (120 mg, 0.203 mmol) in MeOH (5 mL), NaOMe (0.46 mL, 1M, 0.46 mmol) was slowly added at 0° C. The reaction mixture was stirred at room temperature for 2 h. After completion, the reaction mixture was acidified with Amberlyst 15 resin (pH ˜5) filtered, washed with MeOH (3×5 mL) and concentrated in vacuo. The residue was purified by prep HPLC to afford anomeric mixture of of the title compound (GTJC-026-P1, 3 mg, mixture of 4 isomers, GTJC-026-P2, 1 mg, anomeric mixture, a: 3=1:12) as a white solids.

HRMS (ESI) [M+H]+ calc. for C20H21FN4O6S 464.12, found: 465.42 [M+H]+

LCMS: (GTJC-026-P1) m/z 465.4 [M+H]+ (ES+) 58.97% at 4.33 min, 27.97% at 4.38 min, 6.57% at 4.55 min & 2.61% 4.74 min.

1H NMR (GTJC-026-P1) (400 MHz; DMSO-d6, mixture of 4 isomers): └ 8.60 (s, 0.5H), 8.58 (s, 0.5H), 7.87-7.90 (m, 2H), 7.62-7.74 (m, 2H), 7.46-7.61 (m, 4H), 7.12-7.16 (m, 1H), 5.38 (d, 1H, J1-2=7.12 Hz, α-H-1), 3.63-5.39 (m, 7H), 3.10-3.13 (m, 2H), 2.49-2.58 (m, 1H).

LCMS: (GTJC-026-P2) m/z 465.38 [M+H]+, (ES+) 89.0% at 4.33 min and 7.34% at 4.38 min.

1H NMR (GTJC-026-P2) (400 MHz; DMSO-d6, anomeric mixture, α:β=1:12): └ 8.76 (bs, 1H), 8.48 (s, 1H), 7.89 (d, J=7.32, 2H), 7.68-7.72 (m, 2H), 7.46-7.62 (m, 4H), 7.12-7.17 (m, 1H), 5.40 (d, 1H, J1-2=7.12 Hz, α-H-1), 5.26 (d, J=5.7, 1H), 4.83-4.86 (m, 1H), 4.69 (d, J=8.56, 1H), 4.40-4.52 (m, 1H), 3.89-3.90 (m, 1H), 3.62-3.65 (m, 1H), 3.09-3.14 (m, 1H), 2.50 (s, 1H).

### Synthesis of G630

### N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)benzenesulfonamide (GTJC-055)

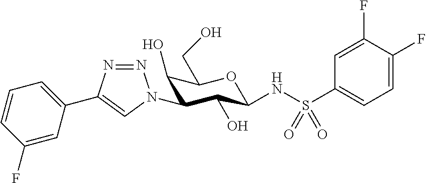

Synthesized following the standard procedure used for GTJC-026

Appearance: White solid

Synthesized: (GTJC-055-P1, 3 isomers, 15 mg) and (GTJC-055-P2, 4 isomers, 8 mg)

HRMS (ESI) [M+H]+ calc. for C20H19F3N4O6S 500.10, found: 501.20 [M+H]+

LCMS (GTJC-055-P1,): m/z 501 [M+H]+ (ES+), 78.32% at 4.66 min, 7.41% at 4.72 min & 12.44% 4.83 min.

1H NMR (GTJC-055-P1) (400 MHz; DMSO-d6): └ 8.55 (s, 1H), 7.81-7.91 (m, 1H), 7.60-7.74 (m, 4H), 7.46-7.51 (m, 1H), 7.12-7.17 (m, 1H), 5.43 (d, 1H, J1-2=7.12 Hz, α-H-1), 5.26 (d, 1H, J=5.88 Hz), 4.83-4.86 (m, 1H), 4.67-4.69 (m, 1H), 4.54-4.57 (m, 1H), 4.01-4.04 (m, 1H), 3.89 (bs, 1H), 3.66-3.69 (m, 1H), 3.32-3.37 (m, 1H), 3.16-3.19 (m, 1H).

LCMS (GTJC-055-P2,): m/z 501 [M+H]+ (ES+), 62.43% at 4.66 min, 24.51% at 4.72 min, 5.63% 4.85 min & 3.81% 5.04 min.

1H NMR (GTJC-055-P2) (400 MHz, DMSO-d6) └ 8.50 (s, 1H), 7.83-7.94 (m, 1H), 7.60-7.74 (m, 5H), 7.46-7.51 (m, 1H), 7.17-7.27 (m, 1H), 5.43 (d, 1H, J1-2=7.2 Hz, α-H-1), 5.24-5.27 (m, 1H), 4.83-4.86 (m, 1H), 4.68 (d, J=8.5 Hz, 1H), 4.46-4.57 (m, 1H), 4.02-4.04 (m, 1H), 3.89 (m, 1H), 3.66-3.67 (m, 1H), 3.31-3.35 (m, 1H), 3.16-3.19 (m, 1H).

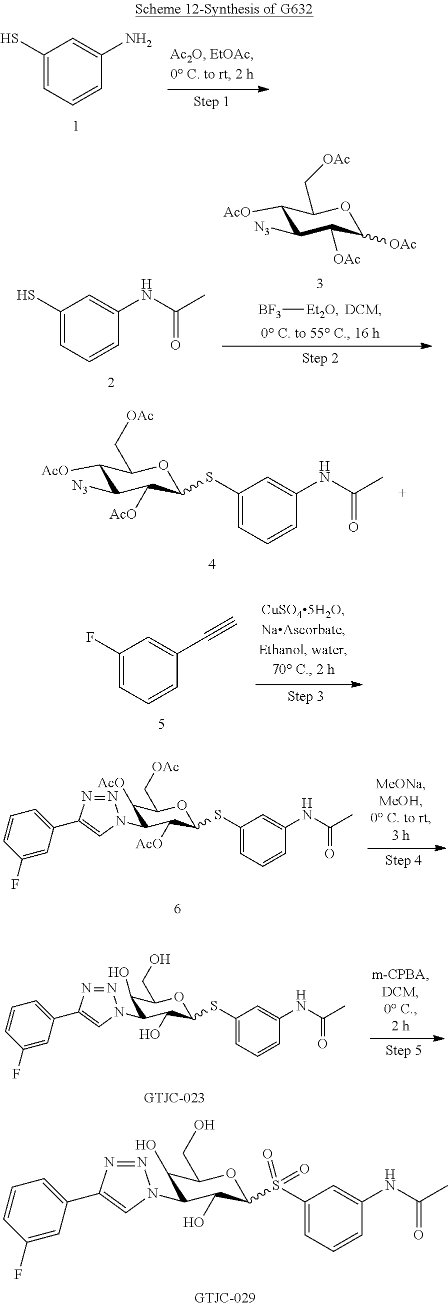

### Step-1

### N-(3-mercaptophenyl)acetamide (2)

To a solution of 3-aminobenzenethiol (2 g, 16.0 mmol) in EtOAc (50 mL), Ac20 (1.66 mL, 17.6 mmol) was slowly added at 0° C. and the reaction mixture was stirred at room temperature for 2 h. After completion, the reaction mixture was quenched with water (40 mL). After separating the organic layer, the aq layer was extracted with EtOAc (2×10 mL). The combined organic layers were washed with brine, dried (Na2SO4) and concentrated in vacuo to afford the title compound as light brown sticky solid (2.23 g, 83%). ESIMS: m/z 166 [M+H]+; 1H NMR (400 MHz, DMSO-d6): └ 2.02 (s, 3H), 5.39 (bs, 1H), 6.94 (d, J=7.6 Hz, 1H), 7.14 (t, J=8.0 Hz, 1H), 7.23 (d, J=8.0 Hz, 1H), 7.60 (s, 1H), 9.90 (s, 1H).

### Step-2

### (3R,4S,5R,6R)-2-((3-acetamidophenyl)thio)-6-(acetoxymethyl)-4-azidotetrahydro-2H-pyran-3,5-diyl diacetate (4)

A solution of N-(3-mercaptophenyl)acetamide (2, 161 mg, 0.96 mmol) and (3R,4S,5R,6R)-6-(acetoxymethyl)-4-azidotetrahydro-2H-pyran-2,3,5-triyl triacetate (180 mg, 0.48 mmol) in DCM (20 mL), BF3.Et2O (304 mg, 0.96 mmol) was slowly added at 0° C. and the reaction mixture was heated at 55° C. for 16 h. After completion, the reaction mixture was quenched with water (10 mL) and extracted with DCM (3×10 mL). The combined organic layers were washed with brine, dried (Na2SO4) and concentrated in vacuo. The crude residue was purified by flash column chromatography [normal phase, silica gel (100-200 mesh), gradient 0% to 70% EtOAc in hexane] to afford the title compound as a off white solid (177 mg, 77%). The crude residue was used for the next step without further purification. ESIMS: m/z 481 [M+H]+

### Step-3

### (3R,4S,5R,6R)-2-((3-acetamidophenyl)thio)-6-(acetoxymethyl)-4-(4-(3-fluorophenyl)-1H-1,2,3-triazol-1-yl)tetrahydro-2H-pyran-3,5-diyl diacetate (6)

To a solution of (3R,4S,5R,6R)-2-((3-acetamidophenyl)thio)-6-(acetoxymethyl)-4-azidotetrahydro-2H-pyran-3,5-diyl diacetate (230 mg 0.48 mmol) and 1-ethynyl-3-fluorobenzene (121 mg, 0.96 mmol) in EtOH (5 mL) and water (5 mL), sodium ascorbate (43 mg, 0.21 mmol) and CuSO4.5H2O (32 mg, 0.07 mmol) were added at room temperature. The reaction mixture was heated to 70° C. for 2 h. After completion, the reaction mixture was diluted with EtOAc (10 mL) and filtered through a pad of celite, washed with EtOAc (3×10 mL), and concentrated in vacuo to afford the title compound as an off white solid (243 mg, 84%). The crude residue was used for the next step without further purification. ESIMS: m/z 601 [M+H]+.

### Step-4

### Synthesis of N-(3-(((3R,4S,5R,6R)-(4-(3-fluorophenyl)-1H-1,2,3-triazol-1-yl)-3,5-dihydroxy-6-(hydroxymethyl)tetrahydro-2H-pyran-2-yl)thio)phenyl)acetamide (GTJC-023)

To a solution of (3R,4S,5R,6R)-2-((3-acetamidophenyl)thio)-6-(acetoxymethyl)-4-(4-(3-fluorophenyl)-1H-1,2,3-triazol-1-yl)tetrahydro-2H-pyran-3,5-diyl diacetate (180 mg, 0.3 mmol) in MeOH (5 mL), NaOMe (0.3 mL, 1M, 0.3 mmol) was slowly added at 0° C. and stirred at rt for 3 h. After completion, the reaction mixture was acidified with Amberlyst 15 resin (pH ˜5), filtered, washed with MeOH (3×10 mL) and concentrated in vacuo. The crude residue was purified by flash column chromatography [normal phase, silica gel (100-200 mesh), gradient 0% to 10% MeOH in DCM]. The white solid obtained was triturated with Et2O to afford the title compound as a white solid (68 mg, 48%).

LCMS: m/z 475 (M+H)+ (ES+), 75.88% at 4.40 min and 22.76% at 4.63 min.

1H NMR (400 MHz; DMSO-d6): └ 2.04 (S, 3H), 3.35-4.05 (overlapping signals, m, 4H), 4.12-5.67 (overlapping signals, m, 6H), 7.23-7.31 (m, 3H), 7.36-7.52 (m, 2H), 7.67-7.76 (m, 2H), 7.80 (s, 1H), 8.69, 8.88 (each singlet, 1H), 9.96, 10.01 (each singlet, 1H).

### Step-5

### N-(3-(((3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)sulfonyl)phenyl)acetamide (GTJC-029)

To a solution of N-(3-(((3R,4S,5R,6R)-4-(4-(3-fluorophenyl)-1H-1,2,3-triazol-1-yl)-3,5-dihydroxy-6-(hydroxymethyl) tetrahydro-2H-pyran-2-yl)thio)phenyl)acetamide (30 mg, 0.063 mmol) in DCM (4 mL), m-CPBA (15 mg, 0.063 mmol) was added at 0° C. and stirred at same temperature for 2 h. After completion, the reaction mixture was quenched with aq. NaOH (10 mL, 2M) and the aq. layer was extracted with DCM (3×10 mL). The combined organic layers were washed with brine, dried (Na2SO4) and concentrated in vacuo. The residue was triturated with Et2O to afford the title compound as a white solid (16 mg, 50%).

LCMS: m/z 475 [M+H]+ (ES+), 22.84% at 4.18 min, 64.79% at 4.29 min, 8.33% at 4.41 min, 2.40% at 4.91 min.

1H NMR (400 MHz, DMSO-d6): └ 2.07, 2.08 (each singlet, 3H), 3.26-3.31 (m, 1H), 3.40-4.74 (overlaping signals, m, 6H), 4.96 (dd, J=10.6 & 2.3 Hz, 1H), 5.22-5.98 (overlaping signals, m, 2H), 7.13-7.17 (m, 1H), 7.46-7.62 (m, 3H), 7.67-7.78 (m, 2H), 7.83-7.88 (m, 1H), 8.20, 8.21, 8.27 (each singlet, 1H), 8.67, 8.68, 8.77 (each singlet, 1H), 10.29, 10.30, 10.34 (each singlet, 1H).

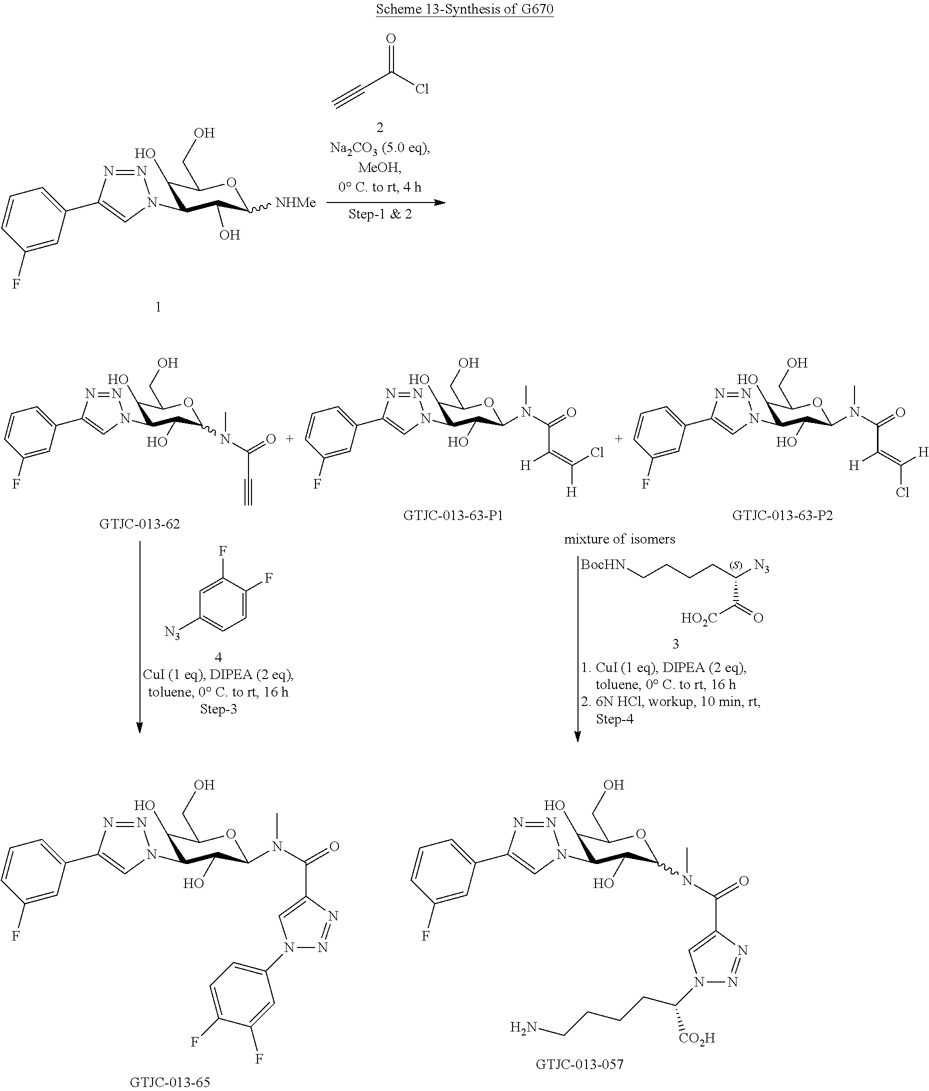

### Steps-1& 2

### N-((3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylpropiolamide (GTJC-013-62)

To a solution of (2R,3R,4S,5R,6R)-4-(4-(3-fluorophenyl)-1H-1,2,3-triazol-1-yl)-2-(hydroxymethyl)-6-(methylamino)tetrahydro-2H-pyran-3,5-diol (300 mg, 0.8875 mmol) in MeOH (10 mL), Na2CO3 (940 mg, 8.8757 mmol) was added and the reaction mixture was cooled to 0° C. propioloyl chloride (156 mg, 1.7751 mmol) was slowly added at 0° C. and the reaction mixture was stirred at room temperature for 2 h. After completion, the reaction mixture was concentrated in vacuo. The crude residue was purified by flash column chromatography [normal phase, silica gel (100-200 mesh), gradient 0 to 10% MeOH in DCM] to give mixture of N-((3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylpropiolamide (GTJC-013-62, single β isomer, 2 mg) as a white solid, (Z)-3-chloro-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylacrylamide (GTJC-013-63-P1, single R isomer, 2 mg) as a white solid and (E)-3-chloro-N-((2R,3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylacrylamide (GTJC-013-63-P2, single R isomer, 1 mg) as a white solid.

HRMS (ESI) [M+H]+ calc. for C18H19FN4O5 390.13, found: 391.16 [M+H]+

LCMS (GTJC-013-62): m/z 391 [M+H]+ (ES+) 95.11% at 3.88 min.

1H-NMR (400 MHz; DMSO-d6, (GTJC-013-62, single β isomer)): └ 8.74 (s, 1H), 7.73-7.75 (m, 1H), 7.68-7.70 (m, 1H), 7.47-7.52 (m, 1H), 7.13-7.17 (m, 1H), 5.66 (d, 1H, J1-2=6.12 Hz, α-H-1), 3.80-5.51 (m, 7H), 3.51-3.53 (m, 2H), 3.15-3.17 (m, 2H), 2.89 (s, 2H).

HRMS (ESI) [M+H]+ calc. for C18H20ClFN4O5 426.11, found: 427.11 [M+H]+

LCMS (GTJC-013-63-P1): m/z 427.1 [M+H]+ (ES+) 93.52% at 3.96 min.

1H NMR (400 MHz, DMSO-d6, (GTJC-013-63-P1, single β isomer)): └ 8.73 (s, 1H), 7.53-7.75 (m, 1H), 7.68-7.70 (m, 1H), 7.47-7.52 (m, 1H), 7.13-7.17 (m, 1H), 6.74-6.76 (m, 1H), 6.65-6.67 (m, 1H), 5.60 (d, 1H, J1-2=8.6 Hz, α-H-1), 5.30-5.37 (m, 1H), 4.91-4.95 (m, 2H), 4.74 (m, 1H), 4.37 (m, 1H), 3.92 (m, 1H), 3.81 (t, J=5.96 Hz, 1H), 3.51 (m, 2H), 2.90 (s, 3H).

HRMS (ESI) [M+H]+ calc. for C18H20ClFN4O5 426.11, found: 427.14 [M+H]+

LCMS (GTJC-013-63-P2): m/z 427.1 [M+H]+ (ES+), 90.74% at 4.22 min.

1H NMR (400 MHz; DMSO-d6) (GTJC-013-63-P2, single β isomer): └ 8.74 (s, 1H), 7.73-7.75 (m, 1H), 7.68-7.71 (m, 1H), 7.47-7.52 (m, 1H), 7.31-7.34 (m, 1H), 7.13-7.17 (m, 1H), 7.09 (m, 1H), 5.59 (m, 1H), 5.38 (m, 1H), 5.22 (d, 1H, J1-2=8.84 Hz, α-H-1), 4.98 (m, 1H), 4.76 (m, 1H), 4.39 (m, 1H), 3.92-3.93 (m, 2H), 3.04 (m, 2H), 2.90 (s, 3H).

### (S)-6-amino-2-(4-(((3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)(methyl)carbamoyl)-1H-1,2,3-triazol-1-yl)hexanoic acid (GTJC-057)

To a solution of N-((3R,4S,5R,6R)-4-(4-(3-fluorophenyl)-1H-1,2,3-triazol-1-yl)-3,5-dihydroxy-6-(hydroxymethyl)tetrahydro-2H-pyran-2-yl)-N-methylpropiolamide (100 mg 0.2564 mmol) in Toluene (5 mL), was added DIPEA (66.15 mg, 0.5128 mmol), CuI (48.50 mg, 0.2564 mmol) and (S)-3-azido-7-((tert-butoxycarbonyl)amino)-2-oxoheptanoic acid (92.7 mg, 0.3076 mmol) at 0° C. The reaction mixture was stirred at room temperature. After 12 h, the reaction mixture was quenched with in HCl (5 mL) then stirred for 30 minutes. Organic compound from the separated aqueous layer was extracted with 5% MeOH in DCM (3×25 mL). The combined organic layers were washed with brine and dried over anhydrous Na2SO4. The solvent was removed under reduced pressure at 45° C. temperature to afford the title compound as a white solid (GTJC-057, single 13 isomer, 15 mg, 5.2%).

HRMS (ESI) [M+H]+ calc. for C24H31FN8O7 562.23, found: 563.38 [M+H]+

LCMS: m/z 563.3 (M+H)+ (ES+) 98.41% at 3.13 min.

1H NMR (400 MHz; DMSO-d6, single β isomer): └ 8.77 (s, 1H), 8.42 (m, 1H), 7.68-7.75 (m, 2H), 7.38-7.57 (m, 1H), 7.12-7.16 (t, J=8.42 Hz, 1H), 5.72 (d, 1H, J1-2=7.8 Hz, α-H-1), 5.64 (m, 1H), 5.25-5.35 (m, 1H), 4.61-4.70 (m, 1H), 4.51 (bs, 1H), 3.93 (m, 1H), 3.71 (m, 1H), 3.54 (m, 2H), 3.31-3.35 (m, 2H), 3.26-3.31 (m, 2H), 3.05 (s, 2H), 2.78 (m, 1H), 2.49-2.54 (m, 2H), 2.44 (s, 1H), 2.13-2.24 (m, 1H), 1.55-1.59 (m, 1H), 1.29 (m, 1H).

TABLE 1
Exemplary compounds according to some embodiments
with inhibition of Insulin Receptor interaction with galectin-3:

| | Inventory |
|---|---|
| Structures | Code |
| 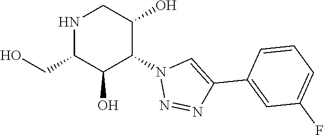 [SMILES: OC[C@@H]1NC[C@H](O)[C@H](n2cc(-c3cccc(F)c3)nn2)[C@H]1O] | G-601 intermediate Isomer 1 |
| 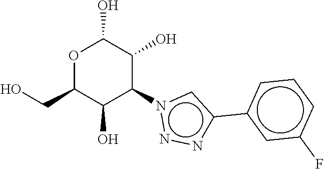 | G-602 intermediate Isomer 2 |
| 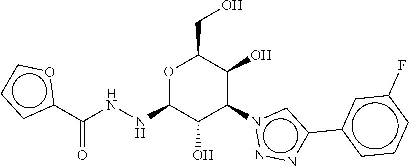 | G-611 |
| 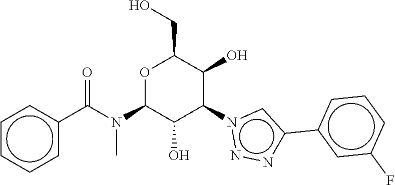 | G-610 |
| 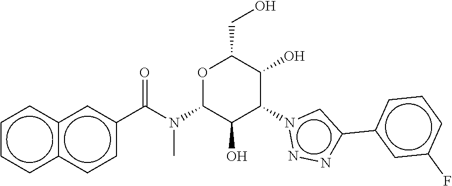 | G-617 |
| 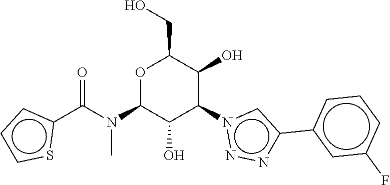 | G-620 |
| 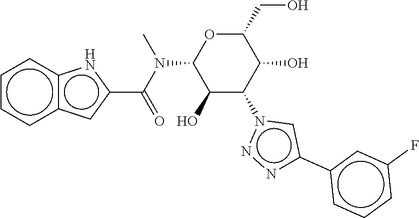 | G-622 |
| 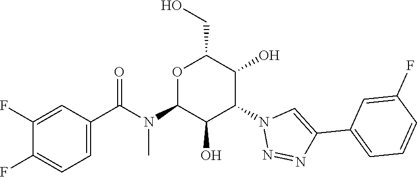 [SMILES: CN(C(=O)c1ccc(F)c(F)c1)[C@H]1O[C@H](CO)[C@H](O)[C@H](n2cc(-c3cccc(F)c3)nn2)[C@H]1O] | G-628 |
| 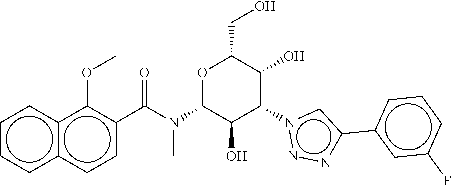 | G-629 |
| 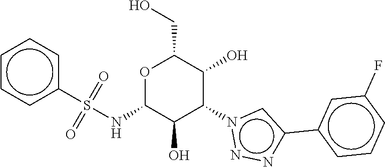 | G-630 |
| 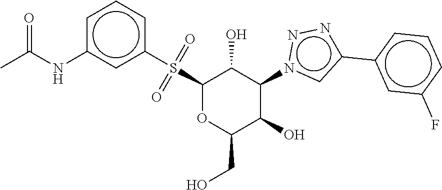 | G-632 |
| 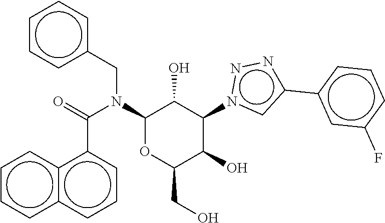 | G-633 |
| 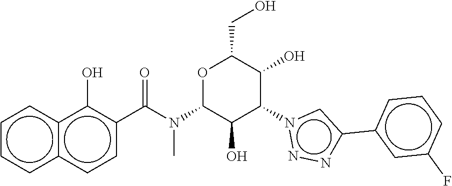 | G-635 |
| 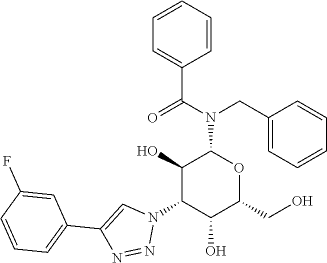 [SMILES: O=C(c1ccccc1)N(Cc1ccccc1)[C@@H]1O[C@H](CO)[C@H](O)[C@H](n2cc(-c3cccc(F)c3)nn2)[C@H]1O] | G-639 |
| 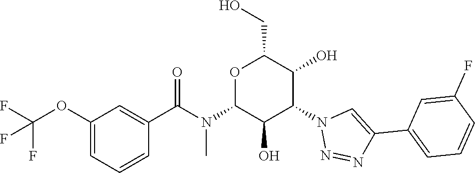 [SMILES: CN(C(=O)c1cccc(OC(F)(F)F)c1)[C@@H]1O[C@H](CO)[C@H](O)[C@H](n2cc(-c3cccc(F)c3)nn2)[C@H]1O] | G-642 |
| 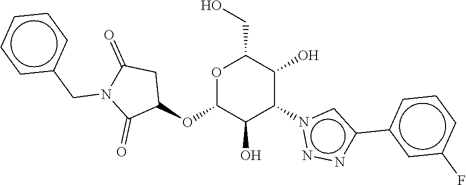 | G-646 |
| 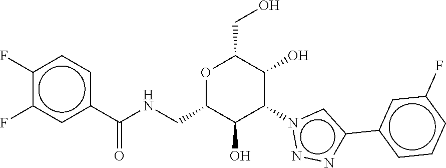 | G-647 |
| 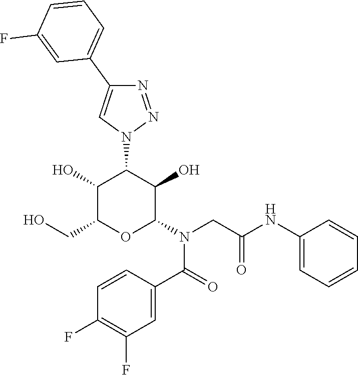 [SMILES: O=C(CN(C(=O)c1ccc(F)c(F)c1)[C@@H]1O[C@H](CO)[C@H](O)[C@H](n2cc(-c3cccc(F)c3)nn2)[C@H]1O)Nc1ccccc1] | G-648 |
| 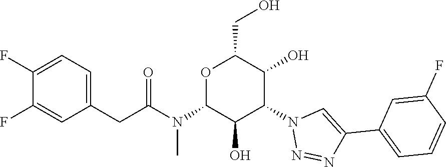 [SMILES: CN(C(=O)Cc1ccc(F)c(F)c1)[C@@H]1O[C@H](CO)[C@H](O)[C@H](n2cc(-c3cccc(F)c3)nn2)[C@H]1O] | G-649 |
| 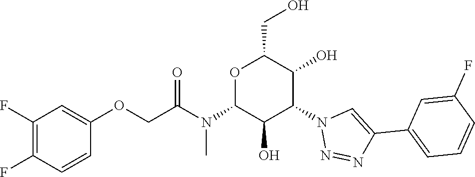 [SMILES: CN(C(=O)COc1ccc(F)c(F)c1)[C@@H]1O[C@H](CO)[C@H](O)[C@H](n2cc(-c3cccc(F)c3)nn2)[C@H]1O] | G-651 |
| 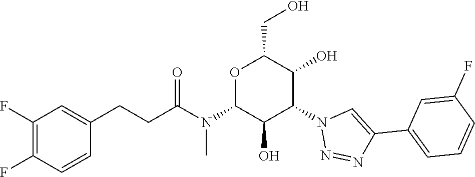 [SMILES: CN(C(=O)CCc1ccc(F)c(F)c1)[C@@H]1O[C@H](CO)[C@H](O)[C@H](n2cc(-c3cccc(F)c3)nn2)[C@H]1O] | G-652 |
| 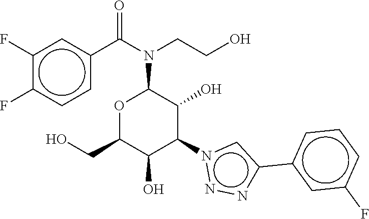 | G-653 |
| 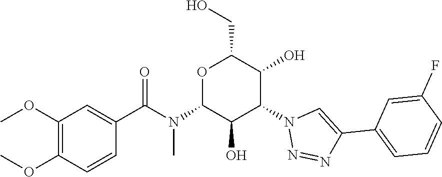 [SMILES: COc1ccc(C(=O)N(C)[C@@H]2O[C@H](CO)[C@H](O)[C@H](n3cc(-c4cccc(F)c4)nn3)[C@H]2O)cc1OC] | G-655 |
| 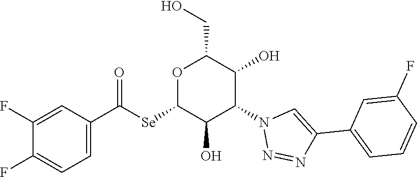 [SMILES: O=C([Se][C@@H]1O[C@H](CO)[C@H](O)[C@H](n2cc(-c3cccc(F)c3)nn2)[C@H]1O)c1ccc(F)c(F)c1] | G-656 |
| 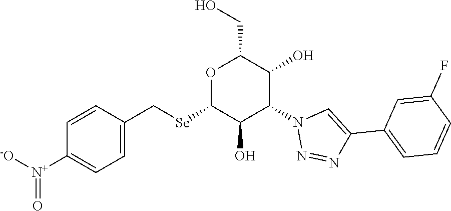 | G-657 |
| 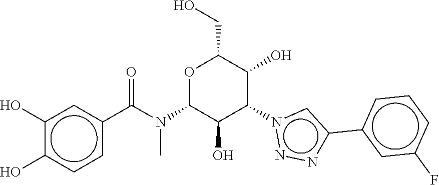 | G-658 |
| 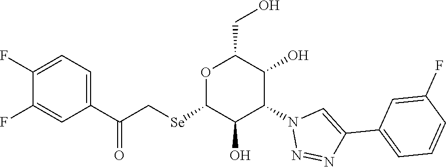 [SMILES: O=C(C[Se][C@@H]1O[C@H](CO)[C@H](O)[C@H](n2cc(-c3cccc(F)c3)nn2)[C@H]1O)c1ccc(F)c(F)c1] | G-662 |
| 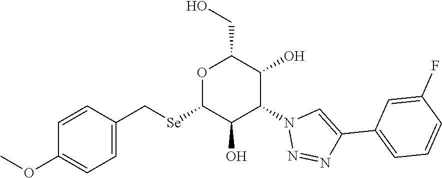 [SMILES: COc1ccc(C[Se][C@@H]2O[C@H](CO)[C@H](O)[C@H](n3cc(-c4cccc(F)c4)nn3)[C@H]2O)cc1] | G-663 |
| 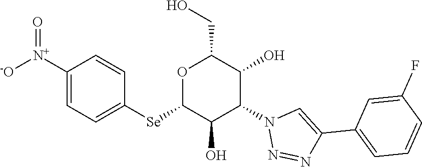 | G-664 |
| 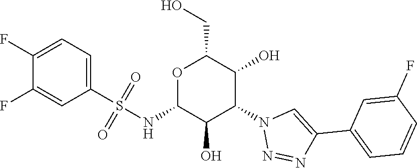 [SMILES: O=S(=O)(N[C@@H]1O[C@H](CO)[C@H](O)[C@H](n2cc(-c3cccc(F)c3)nn2)[C@H]1O)c1ccc(F)c(F)c1] | G-666 |
| 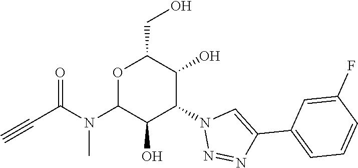 [SMILES: C#CC(=O)N(C)C1O[C@H](CO)[C@H](O)[C@H](n2cc(-c3cccc(F)c3)nn2)[C@H]1O] | G-667 |
| 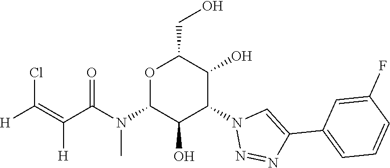 [SMILES: CN(C(=O)/C=C\Cl)[C@@H]1O[C@H](CO)[C@H](O)[C@H](n2cc(-c3cccc(F)c3)nn2)[C@H]1O] | G-668 |
| 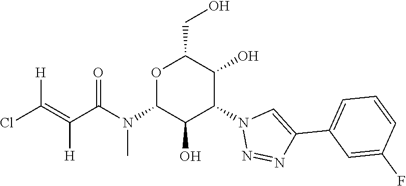 [SMILES: CN(C(=O)/C=C/Cl)[C@@H]1O[C@H](CO)[C@H](O)[C@H](n2cc(-c3cccc(F)c3)nn2)[C@H]1O] | G-669 |
| 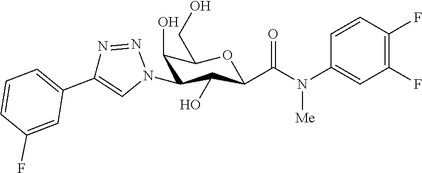 | G-678 |
| 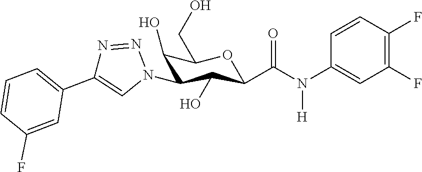 | G-678 |
| 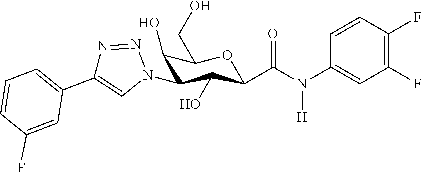 | G-679 |
| 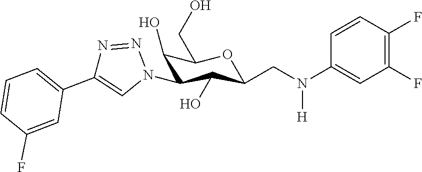 | G-680 |

1. A method for treatment of metabolic diseases associated with systemic insulin resistance comprising administering to a subject in need thereof a therapeutically effective amount of a compound of Formula 1 or a pharmaceutically acceptable salt or solvate thereof: 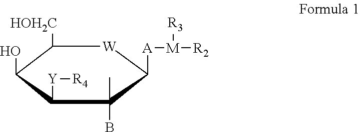
wherein A is independently selected from the group consisting of NRa, CRb, and PRc,
wherein M is independently selected from the group consisting of NRa, CRb, PRc, ORd, SRe amino acid, and hydrophobic hydrocarbons derivatives including heterocyclic substitutions of 3 or more atoms,
wherein Ra is selected from the group consisting of H, H2, CH3, COOH, NH2, COMe, halogen and combinations thereof,
wherein Rb is selected from the group consisting of H, H2, O, OH, CH3, COOH, NH2, COMe, halogen and combinations thereof,
wherein Rc is selected from the group consisting of O2, PO2, OH, halogen and combinations thereof,
wherein Rd is selected from the group consisting of H, CH3, and combination thereof,
wherein Re is selected from the group consisting of OH, O2, S, halogen and combinations thereof,
wherein B is OH, NH2, NHAc, or NH-alkyl, wherein the alkyl group comprises 1 to 18 Carbons,
wherein W is selected from the group consisting of O, S, CH2, NH, and Se,
wherein Y is selected from the group consisting of O, S, NH, CH2, Se, S, P, amino acid, and hydrophobic linear and cyclic hydrophobic hydrocarbons derivatives including heterocyclic substitutions of molecular weight of about 50-200 D and combinations thereof,
wherein R₁, R₂, and R₃ are independently selected from the group consisting of H, O2, CO, NH2, SO2, SO, PO2, PO, CH3, linear hydrocarbon, and cyclic hydrocarbon, and
wherein the hydrocarbon is one of a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, Deoxygalactose, substituted D-Galctose, C3-[1,2,3]-triaZol-1-yl-substituted D-galactose, hydrogen, an alkyl group, an alkenyl group, an aryl group, a heteroaryl group, and a heterocycle and derivatives, an amino group, a substituted amino group, an imino group, or a substituted imino group.

2. The method of claim 1, wherein the A-M spacer represents a spacer of at least 2 atoms comprising an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, Hydrazide —N(—H)—N(—H)—, amino acid, or combinations thereof.

3. The method of claim 1, wherein the A-M spacer is linked to the anomeric carbon and comprise of an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, carbohydrazide —C(═O)—NH—NH—, sulfonohydrazide —S(═O)2-NH—NH—, and phosphonic dihydrazide —P(═O)(—NH—NH2)(NH—NH—) spacer.

4. The method of claim 1, wherein the A-M spacer comprises 2 or more atoms linked by single or double bond: C—C, C═C, C—P, C—N, C—O, N—C, N—N, N═N, N—S, N—P, S—N, P—O, O—P, or combination thereof.

5. The method of claim 1, wherein the A-M spacer comprises PO2 or PO2-PO2 bond linked to the anomeric carbon and to one or more atoms such as C or N or O or S.

6. The method of claim 5, wherein C or N is linked to the anomeric carbon and PO2 or PO2-PO2 is linked to C or N.

7. The method of claim 1, wherein the A-M linked to R1 and R2 is N′-methylamide-3,4-difluorobenzene and Y—R₁ is triazole-3-fluorobenzene:

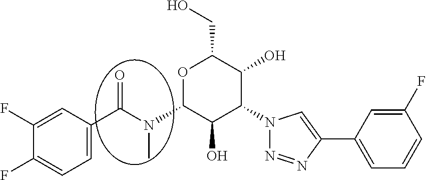

8. The method of claim 1, wherein the A-M spacer is linked to a galactose, a hydroxyl cyclohexane, an aromatic moiety, an alkyl group, an aryl group, an amine group, or amide group.

9. The method of claim 1, wherein the A-M spacer symmetrically links two galactosides or substituted derivatives thereof.

10. The method of claim 1, wherein the A-M spacer asymmetrically links two galactosides or substituted derivatives thereof.

11. The method of claim 1, wherein the anomeric carbon of the galactoside has a spacer of 2 or more atoms linked by single or double bond: C—C, C═C, C—P, C—N, C—O, N—C, N—N, N═N, N—S, N—P, S—N, P—O, O—P, or combination thereof.

12. A method for treatment of systemic insulin resistance comprising administering to a subject in need thereof a therapeutically effective amount of a compound of Formula (2) or a pharmaceutically acceptable salt or solvate thereof 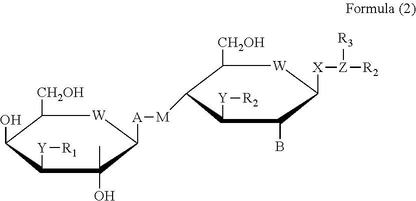
wherein A is independently selected from the group consisting of NRa, CRb, and PRc,
wherein M is independently selected from the group consisting of NRa, CRb, PRc, ORd, SRe amino acid, and hydrophobic hydrocarbons derivatives including heterocyclic substitutions of 3 or more atoms.
wherein A is independently selected from the group consisting of NRa, CRb, and PRc
wherein M is independently selected from the group consisting of NRa, CRb, PRc, ORd, SRe amino acid, and hydrophobic hydrocarbons derivatives including heterocyclic substitutions of 3 or more atoms,
wherein Ra is selected from the group consisting of H, H2, CH3, COOH, NH2, COMe, halogen and combinations thereof,
wherein Rb is selected from the group consisting of H, H2, O, OH, CH3, COOH, NH2, COMe, halogen and combinations thereof,
wherein Rc is selected from the group consisting of O2, PO2, OH, halogen and combinations thereof,
wherein Rd is selected from the group consisting of H and CH3,
wherein Re is selected from the group consisting of OH, O2, S, halogen and combinations thereof,
wherein B is OH, NH2, NHAc, or NH-alkyl, wherein the alkyl group comprises 1 to 18 Carbons,
wherein B is OH, NH2, NHAc, or NH-alkyl wherein the alkyl comprises 1 to 18 Carbons,
wherein W is selected from the group consisting of O, S, CH2, NH, and Se,
wherein X is selected from the group consisting of O, N, S, CH2, NH, and PO2,
wherein Y and Z are independently selected from the group consisting of O, S, C, NH, CH2, Se, S, P, amino acid, and hydrophobic linear and cyclic hydrophobic hydrocarbons derivatives including heterocyclic substitutions of molecular weight of about 50-200 D and combinations thereof,
wherein R1, R2, R3, are independently selected from the group consisting of CO, O2, SO2, SO, PO2, PO, CH, Hydrogen, hydrophobic linear hydrocarbon, and hydrophobic cyclic hydrocarbon, wherein the hydrocarbon is one of:
a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted With an amino group, an alkyl group of at least 3 carbons substituted with both an amino and a carboxy group, an alkenyl group of at least 3 carbons substituted with both an amino and a carboxy group, and an alkyl group substituted with one or more halogens;
b) a phenyl group substituted with at least one car boxy group, a phenyl group substituted With at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group,
c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group; and
d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group,
e) saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, or a substituted imino group.

13. The method of claim 12, wherein A-M represents a spacer of at least 2 atoms comprising an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, Hydrazide —N(—H)—N(—H)—, or combinations thereof.

14. The method of claim 12, wherein A-M represents a spacer linked to the anomeric carbon and comprising of an amide —N(—Ra)—C(═O)—, sulfonamide —N(—H)—S(═O2)-, a methylether —C(—H2)-O— methylester —C(═O)—O—, carbosulfon —C(—H2)-S(═O)(═O)—, phosphate —O—P(═O)(—OH)—, diphosphate —O—P(═O)(—O)—O—P(═O)(—O)—, carbohydrazide —C(═O)—NH—NH—, sulfonohydrazide —S(═O)2-NH—NH—, and phosphonic dihydrazide —P(═O)(—NH—NH2)(NH—NH—) spacer.

15. The method of claim 12, wherein A-M represents a spacer comprising 2 or more atoms linked by single or double bond: C—C, C═C, C—P, C—N, C—O, N—C, N—N, N═N, N—S, N—P, S—N, P—O, O—P, or combination thereof.

16. The method of anyone of claims 1-15, wherein the A-M spacer of at least 2 atoms has a rotational freedom and length configured to allow an interaction of about 1 nM to about 50 μM to a galectin CRD epitope.

17. The method of anyone of claims 1-15, wherein the hydrophobic linear and cyclic hydrocarbons including heterocyclic substitutions have a molecule weight of about 50 to 200 D.

18. A method for treatment of systemic insulin resistance comprising administering to a subject in need thereof a therapeutically effective amount of a compound of Table 1 or a pharmaceutically acceptable salt or solvate thereof.

19. The method of anyone of claims 1-15 or 18, wherein the compound is in a crystalline form.

20. The method of anyone of claims 1-15 or 18, wherein the compound is in a free form.

21. The method of anyone of claim 1-15 or 18, wherein the free form is an anhydrate.

22. The method of anyone of claim 1-15 or 18, wherein the free form is a hydrate.

23. The method of anyone of claims 1-15 or 18, wherein the compound binds Galectin 3, Galectin 1, Galectin 8, Galectin 9 or combinations thereof.

24. The method of anyone of claims 1-15 or 18, wherein the compound has an affinity of about 1 nM to about 50 μM for Galectin-3.

25. The method of anyone of claims 1-15 or 18, comprising administering a therapeutically effective amount of the compound and a pharmaceutically acceptable adjuvant, excipient, formulation carrier or combinations thereof.

26. The method of anyone of claims 1-15 or 18, comprising administering a therapeutically effective amount of the compound and a therapeutically effective amount of an anti-inflammatory drug, vitamin, pharmaceutical drug, nutraceutical drug, supplement, or combinations thereof.

27. A method for treatment of systemic insulin resistance comprising administering to a subject in need thereof a therapeutically effective amount of a compound of Formula (3) or a pharmaceutically acceptable salt or solvate thereof 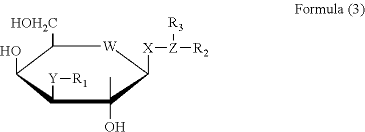
wherein X is S, O, or S(O2),
wherein W is selected from the group consisting of O, N, S, CH2, NH, and Se,
wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms,
wherein R₁, R₂, and R₃ are independently selected from the group consisting of CO, O2, SO2, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide; a substituted saccharide, D-galactose, substituted D-galactose, C3-[1,2,3]-triazol-1-yl-substituted D-galactose, hydrogen, an alkyl group, an alkenyl group, an aryl group, a heteroaryl group, and a heterocycle and derivatives, an amino group, a substituted amino group, an imino group, and a substituted imino group.

28. A method for treatment of systemic insulin resistance comprising administering to a subject in need thereof a therapeutically effective amount of a compound of Formula (4) or a pharmaceutically acceptable salt or solvate thereof 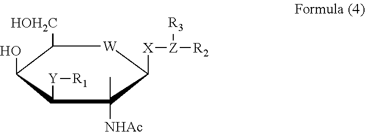
wherein X is S, O, or S(O2),
wherein W is selected from the group consisting of O, N, S, CH2, NH, and Se,
wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms,
wherein R₁, R₂, and R₃ are independently selected from the group consisting of CO, O2, SO2, PO2, PO, CH, Hydrogen, or combination of these and, a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives; an amino group, a substituted amino group, an imino group, and a substituted imino group.

29. A method for treatment of systemic insulin resistance comprising administering to a subject in need thereof a therapeutically effective amount of a compound of general Formula (5) or a pharmaceutically acceptable salt or solvate thereof 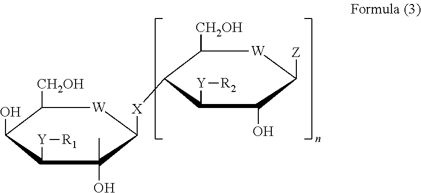
wherein X is S, O, or S(O2),
wherein W is selected from the group consisting of O, N, S, CH2, NH, and Se,
wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms,
wherein n≤24,
wherein R₁ and R₂ are independently selected from the group consisting of CO, O2, SO2, SO, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one car boxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted With at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, or a substituted imino group.

30. A method for treatment of systemic insulin resistance comprising administering to a subject in need thereof a therapeutically effective amount of a compound of general Formula (6) or a pharmaceutically acceptable salt or solvate thereof 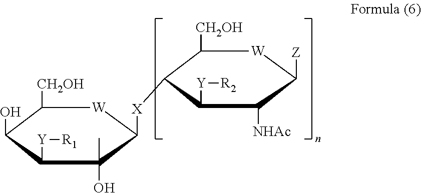
wherein X is S, O, or S(O2),
wherein W is selected from the group consisting of O, N, S, CH2, NH, and Se,
wherein Y is selected from the group consisting of O, S, NH, CH2, Se, S, S(O2), P(O2), amino acid, an hydrophobic linear and cyclic hydrophobic hydrocarbons derivatives including heterocyclic substitutions of molecular weight of about 50-200 D and combinations thereof,
wherein Z is selected from the group consisting of O, S, NH, CH2, Se, P(O2), and hydrophobic hydrocarbons derivatives including heterocyclic substitutions of 3 or more atoms,
wherein n≤24,
wherein R₁ and R₂ are independently selected from the group consisting of CO, O2, SO2, SO, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one car boxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triaZol-1-yl-substituted D-galactose, hydrogen, an alkyl group, an alkenyl group, an aryl group, a heteroaryl group, and a heterocycle and derivatives; an amino group, a substituted amino group, an imino group, and a substituted imino group.

31. The method of claim 29 or 30, wherein n=1.

32. The method of claim 29 or 30, wherein n=3.

33. A method for treatment of systemic insulin resistance comprising administering to a subject in need thereof a therapeutically effective amount of a compound of general Formula (7) or a pharmaceutically acceptable salt or solvate thereof 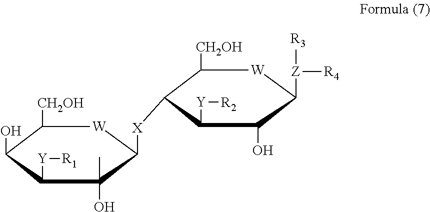
wherein X is S, O, S(O2), S—S, S—S(O2), S(O2)-S, O—S, S—O, O—S(O2), S(O2)-O, O—N(H), O—C(H2), O—C(O), O—C(H,OH), S—N(H), S—C(H2), S(O)—N(H), S(O2)-N(H), or O—P(O2),
wherein W is selected from the group consisting of O, N, S, CH2, NH, and Se,
wherein Y is selected from the group consisting of O, S, C, NH, CH2, Se, P, amino acid, an hydrophobic linear and cyclic hydrophobic hydrocarbons derivatives including heterocyclic substitutions of molecular weight of about 50-200 D and combinations thereof,
wherein Z is selected from the group consisting of O, S, N, CH, Se, S, P, and hydrophobic hydrocarbons derivatives including heterocyclic substitutions of 3 or more atoms,
wherein R₁, R₂, R₃ and R₄ are independently selected from the group consisting of CO, O2, SO2, SO, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide, a substituted saccharide, D-galactose, substituted D-galactose, C3-[1,2,3]-triazol-1-yl-substituted D-galactose, hydrogen, an alkyl group, an alkenyl group, an aryl group, a heteroaryl group, and a heterocycle and derivatives, an amino group, a substituted amino group, an imino group, and a substituted imino group.

34. A method for treatment of systemic insulin resistance comprising administering to a subject in need thereof a therapeutically effective amount of a compound of general Formula (8) or a pharmaceutically acceptable salt or solvate thereof 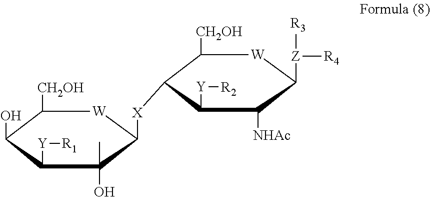
wherein X is S, O, S(O2), S—S, S—S(O2), S(O2)-S, O—S, S—O, O—S(O2), S(O2)-O, O—N(H), O—C(H2), O—C(O), O—C(H,OH), S—N(H), S—C(H2), S(O)—N(H), S(O2)-N(H), or O—P(O2),
wherein W is selected from the group consisting of O, N, S, CH2, NH, and Se,
wherein Y is selected from the group consisting of O, S, C, NH, CH2, Se, amino acid an combinations thereof,
wherein Z is selected from the group consisting of O, S, N, CH, Se, S, P, and hydrophobic hydrocarbons derivatives including heterocyclic substitutions of 3 or more atoms,
wherein R₁, R₂, R₃ and R₄ are independently selected from the group consisting of CO, O2, SO2, SO, PO2, PO, CH, Hydrogen, or combination of these and a) an alkyl group of at least 3 carbons, an alkenyl group of at least 3 carbons, an alkyl group of at least 3 carbons substituted with a carboxy group, an alkenyl group of at least 3 carbons substituted with a carboxy group, an alkyl group of at least 3 carbons substituted with an amino group, an alkenyl group of at least 3 carbons substituted with an amino group, an alkyl group of at least 3 carbons substituted with both an amino and a carboxy group, an alkenyl group of at least 3 carbons substituted with both an amino and a carboxy group, and an alkyl group substituted with one or more halogens, b) a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one dialkylamino group, a phenyl group substituted with at least one hydroxy group, a phenyl group substituted with at least one carbonyl group and a phenyl group substituted with at least one substituted carbonyl group, c) a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one dialkylamino group, a naphthyl group substituted with at least one hydroxy group, a naphthyl group substituted with at least one carbonyl group and a naphthyl group substituted with at least one substituted carbonyl group, d) a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one hydroxy group, a heteroaryl group substituted with at least one carbonyl group and a heteroaryl group substituted with at least one substituted carbonyl group, and e) a saccharide; a substituted saccharide; D-galactose; substituted D-galactose, C3-[1,2,3]-triazol-1-yl-substituted D-galactose; hydrogen, an alkyl group, an alkenyl group, an aryl group, a heteroaryl group, and a heterocycle and derivatives; an amino group, a substituted amino group, an imino group, and a substituted imino group.

35. The method of any of claims 1, 12, 18, 27-30 or 33-34, wherein the halogen is a fluoro, a chloro, a bromo or an iodo group.

36. The method of any of claims 27-30 or 33-34, wherein the compound has a binding affinity for galectins.

37. The method of any of claims 27-30 or 33-34, wherein the compound has a binding affinity for galectin-3.

38. The method according to any of claims 1, 12, 18, 27-30 or 33-34, wherein the step of administering comprises administering the compound and a pharmaceutically acceptable adjuvant, excipient, formulation carrier or combinations thereof.

39. The method of any of claims 1, 12, 18, 27-30 or 33-34, wherein in the step of administering, the compound is administered in conjunction with an active agent.

40. The method of any of claims 1, 12, 18, 27-30 or 33-34, wherein the step of administering comprises administering the compound, a synergistic active agent and a pharmaceutically acceptable adjuvant, excipient, formulation carrier or combinations thereof.

41. The method of any of claims 1, 12, 18, 27-30 or 33-34, wherein the step of administering comprises administering the compound comprises and an active agent, wherein the active agent is an immunomodulatory, an anti-inflammatory drug, a vitamin, a nutraceutical drug, a supplement, or combinations thereof.

42. The method of any of claims 1, 12, 18, 27-30 or 33-34, for treating systemic insulin resistance associated with type 1 diabetes.

43. The method of any of claims 1, 12, 18, 27-30 or 33-34, for treating systemic insulin resistance associated with type 2 diabetes mellitus (T2DM).

44. The method of any of claims 1, 12, 18, 27-30 or 33-34, for treating systemic insulin resistance associated with obesity, gestational diabetes or prediabetes.

45. The method of any of claims 1, 12, 18, 27-30 or 33-34, wherein treatment with the compound restores sensitivity of cells to insulin activity.

46. The method of any of claims 1, 12, 18, 27-30 or 33-34, wherein the compound inhibits galectin-3 interaction with Insulin receptor, thereby interfering with insulin binding and cellular glucose uptake mechanism.

47. The method of any of claims 1, 12, 18, 27-30 or 33-34, for treating of low-grade inflammation, due to elevated levels of free fatty acid and triglycerides that cause insulin resistance in skeletal muscle and liver which contributes to the development of atherosclerotic vascular diseases and NAFLD.

48. The method of any of claims 1, 12, 18, 27-30 or 33-34, for treating polycystic ovarian syndrome (PCOS) associated with obesity, insulin resistance.

49. The method of any of claims 1, 12, 18, 27-30 or 33-34, for treating diabetic nephropathy and glomerulosclerosis by attenuating integrin and TGFb Receptor pathway in kidney chronic disease.

50. The method according to any of claims 1, 12, 18, 27-30 or 33-34, wherein the compound inhibits overexpression of TGF-β receptor signaling system triggered by Insulin resistance in diabetic and causes decline in renal function, and/or wherein the compound reverses established lesions of diabetic glomerulopathy.

51. The method according to any of claims 1, 12, 18, 27-30 or 33-34, for treating obstructive sleep apnea (OSA) associated with insulin resistance obesity and diabetes.

52. The method according to any of claims 1, 12, 18, 27-30 or 33-34, wherein the step of administering comprises administering the compound and a synergistic active antidiabetic drug.
